(19) United States
(12) Patent Application Publication
(10) Pub. No.: US 2025/0213736 A1
(43) Pub. Date: Jul. 3, 2025

(54) SCISSION-ENHANCED NUCLEAR IMAGING AND TREATMENT

(71) Applicant: The General Hospital Corporation, Boston, MA (US)

(72) Inventors: Ralph Weissleder, Peabody, MA (US); Jonathan C. Carlson, Boston, MA (US)

(21) Appl. No.: 18/850,352

(22) PCT Filed: Apr. 17, 2023
(86) PCT No.: PCT/US2023/065862

§ 371 Date: Sep. 24, 2024

Related U.S. Application Data
(60) Provisional application No. 63/331,495, filed on Apr. 15, 2022.

(51) Int. Cl. A61K 51/10; A61K 51/04

(52) U.S. Cl. CPC A61K 51/1093; A61K 51/0497; A61K 2121/00; A61K 2123/00

(57) ABSTRACT

The present disclosure provides compounds and methods for “scission-enhanced” nuclear imaging and treatment (“SENIT”). In one example, the disclosure provides a conjugate where an affinity ligand (e.g., an antibody) is connected to a radionuclide (e.g., a DOTA-chelated ⁶⁸Ga) using a “click-to-release” bioorthogonal linker (e.g., a linker containing a releasable trans-cyclooctene moiety in its structure). As described herein, the imaging and theranostic methods of this disclosure advantageously allow for rapid corporeal elimination of radionuclides once imaging or theranostic treatment is completed.

### CLAIM OF PRIORITY

This application claims priority to U.S. Provisional Patent Application Ser. No. 63/331,495, filed on Apr. 15, 2022, the entire contents of which are hereby incorporated by reference.

### TECHNICAL FIELD

This invention relates to conjugates useful as radiotheranostics and/or as nuclear imaging agents (e.g., for use in PET or SPECT imaging). For example, a conjugate of this invention includes an affinity ligand (e.g., an antibody) attached to a radionuclide (e.g., ⁶⁴Cu, ⁸⁹Zr, or ⁶⁸Ga) using a linker containing a trans-cyclooctene (“TCO”) moiety capable of releasing the radionuclide from the conjugate upon reaction with a tetrazine (“Tz”) antidote. Suitable examples of TCO moieties useful as linkers in the conjugates of this disclosure include trans-cyclooctenes modified in allylic position, such as release-TCO (“rTCO”), cleavable TCO (“cTCO”), 1,4-dihydroxyl TCO (“iTCO”), C₂-symmetric TCO (“C₂TCO”), and cyclopropane-fused analogs (e.g., sTCO, siTCO, and sC₂TCO), dioxolane-fused analogs (e.g., dTCO and dC₂TCO), as well as aziridine-fused analogs (e.g., aza-C₂TCO).

### BACKGROUND

Nuclear imaging methods such as positron emission tomography (“PET”), single-photon emission computed tomography (“SPECT”), and planar gamma imaging have become powerful tools in medicine to visualize diseases and make therapeutic decisions. In addition, β-emitting radionuclides are also increasingly used for therapeutic purposes, e.g., in cancer treatments as radiotheranostics.

### SUMMARY

Positron emission tomography (PET) has enjoyed widespread clinical use following the introduction of hybrid imaging techniques (e.g., PET-CT and PET-MRI) and a plethora of ¹⁸F-labeled small-molecule imaging probes over the last three decades. More recently, immunoPET has developed due to the increasingly rapid approval of therapeutic antibodies and increased production of longer half-life radionuclides (e.g., ⁶⁴Cu, ⁸⁹Zr, or ⁶⁸Ga) useful in PET imaging. Similarly, therapeutic applications of β- and α-emitters (e.g., ⁹⁰Y, ¹⁷⁷Lu, or ²²⁵Ac) have rapidly expanded with the demonstration of clinical efficacy in several oncologic contexts and ensuing FDA approvals. A number of different radionuclide-antibody conjugation strategies have been explored, both for the assembly of the conjugates and functional modulation. Most antibody-based radionuclides have long circulation times and radiation dosing concerns limit the number of scans that can be performed in a patient per year (e.g., no more than three PET scans per year to limit patient's exposure to radiation). Similarly, most theranostics require renal protection programs to limit renal toxicity. One way to reduce unnecessary radiation exposure and improve target-to-background ratios is by “pre-targeting” with separate delivery of the affinity ligand (e.g., the antibody) and the radioligand. However, this approach is often complex and has met varied success in clinical translation.

The disclosure is based, at least in part, on a realization that conjugates containing an affinity ligand (e.g., a cancer-targeting antibody) connected to a radionuclide by a bioorthogonal “releasable” linker are useful in vivo and are non-toxic to patients at concentrations that are necessary for such a use. In contrast to the existing and cumbersome “pre-targeting” methodologies, the conjugates and methods of this disclosure may be advantageously used to “turn-off” radioactive materials on demand. This can be accomplished, for example, by administering a bioorthogonal antidote reagent following the conjugate, leading to cleavage of the bioorthogonal linker and release of the radionuclide to circulation for rapid corporeal elimination. The conjugates within the present claims therefore advantageously provide useful medical strategy to reduce radiotoxicity, to enable more frequent imaging, or to perform multiplexed nuclear imaging, e.g., using different affinity ligands or different imaging modality.

FIG. 1A provides a schematic example showing that bioorthogonal cleavage of radiolabels enables superior methods for imaging and therapeutics. Compared to the current methods where one has to wait for radioactivity to decay within the body (leading to radiotoxicity), the methods illustrated in FIG. 1A enables precision release and excretion of unwanted radiotracers following the imaging or therapeutic protocol. As shown in FIG. 1A, an affinity label (such as antibody, nanobody, aptamer, protein, peptide, oligo, nanoparticle, or a similar tag capable to targeting a particular cell, organ, or tissue) is functionalized with a linker incorporating an immolative TCO moiety such as C₂TCO or iTCO (see inset). This construct is chemically and physically stable and can be used in vivo, e.g., by administering to a patient for imaging and/or therapy. When a dose reduction is desired, a second IV injection of a “tetrazine scissors” antidote can be performed. Without being bound by any theory, the tetrazine (“Tz”) antidote engages in inverse electron demand Diels-Alder (“IEDDA”) reaction with the TCO fragment within the linker, which cleaves the radiolabel-chelator complex from the affinity ligand in a matter of seconds. The radionuclide is then rapidly cleared by the kidneys resulting in dose reduction and favorable imaging/therapy kinetics. An example of the “chemical scissor” reaction cleaving the radionuclide from the conjugate is schematically illustrated in FIG. 1A and also in Scheme 1.

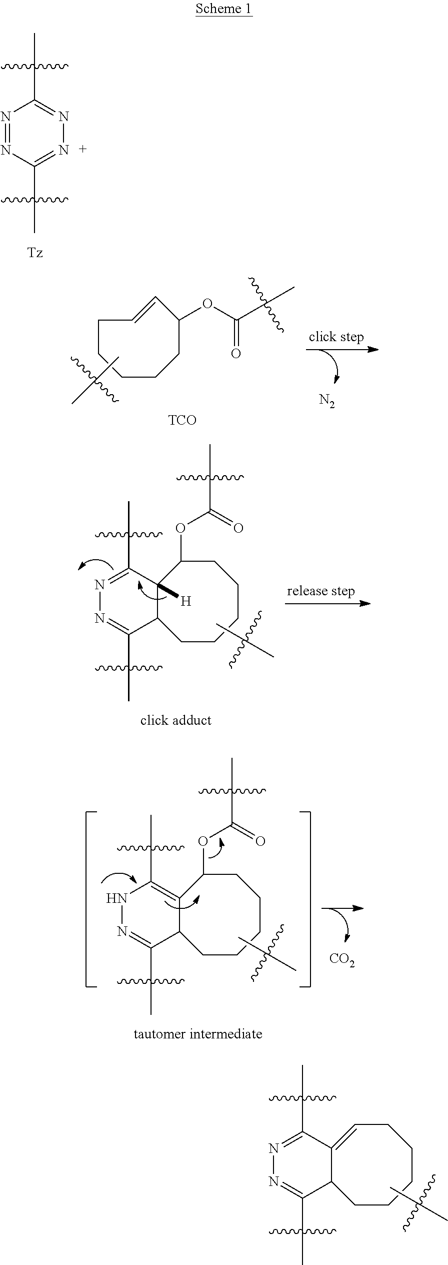

Referring to Scheme 1, a Tz reagent reacts with a radiolabeled affinity ligand containing a TCO fragment in its structure, thereby forming a “click” adduct during the initial “click” step of the reaction. During the second (release) step, the click adduct first tautomerizes to form a tautomer intermediate. The tautomer intermediate then undergoes spontaneous decomposition reaction leading to molecular disassembly of the conjugate, release of the radionuclide, and its “deactivation” by elimination from the patient's body. In one general aspect, incorporation of cleavable TCO linkers into radiolabeled affinity ligands enables precise spatiotemporal control of their properties. Importantly, the methods within the present claims achieve both fast (i.e., instantaneous, or seconds to minutes) and complete (>99%) cleavage and subsequent elimination of radionuclides. By selecting a specific combination of the TCO fragment in the linker and the Tz fragment in the antidote, independent control of click rates (first, click step) and cleavage rates (second, release step) can be achieved, which supports broad utility of the conjugates, e.g., in imaging and/or medical treatments.

In one general aspect, the present disclosure provides a compound of Formula (I):

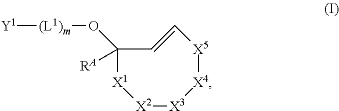

or a pharmaceutically acceptable salt thereof, wherein:
X¹ is selected from O and CR^{A}R¹;
X² is selected from O and CR^{A}R²;
X³ is selected from O and CR^{A}R³;
X⁴ is selected from O and CR^{A}R⁴;
X⁵ is selected from O and CR^{A}R⁵;
each R^{A} is independently selected from H, OH, C₁₋₃ alkyl, and C₁₋₃ haloalkyl; R¹, R², R³, R⁴, and R⁵ are each independently selected from H, halo, C₁₋₆ alkyl, C₁₋₆ haloalkyl, C₁₋₃ alkoxy, C₁₋₃ haloalkoxy, and a moiety of formula (L²)ₙ-(Y²)ₒ—(Y³)ₚ;
or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(═O)OH, C(═O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L²)ₙ-(Y²)ₒ—(Y³)ₚ;
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(═O)OH, C(═O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L²)ₙ-(Y²)ₒ—(Y³)ₚ;
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(═O)OH, C(═O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L²)ₙ-(Y²)ₒ—(Y³)ₚ;
or R⁴ and R⁵, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(═O)OH, C(═O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L²)ₙ-(Y²)ₒ—(Y³)ₚ;
each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(═O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
each L² is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(═O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
each x is independently an integer from 1 to 2,000;
each R^{N} is independently selected from H, C₁₋₃ alkyl, and C₁₋₃ haloalkyl;
Y¹ is selected from NH₂, OH, C(═O)OH, a protected amino group, a protected hydroxyl group, a protected carboxyl group, a reactive chemical group (e.g., a chemical group reactive with an affinity ligand), and an affinity ligand;
m is an integer from 1 to 20;
n is an integer from 1 to 20;
o is 0, 1, 2, or 3;
p is 0, 1, 2, or 3;
Y² is a chelating moiety; and
Y³ is a radiotracer,
provided that at least one of o and p is other than 0.

In some embodiments, the compound of Formula (I) comprises any one of the following moieties:

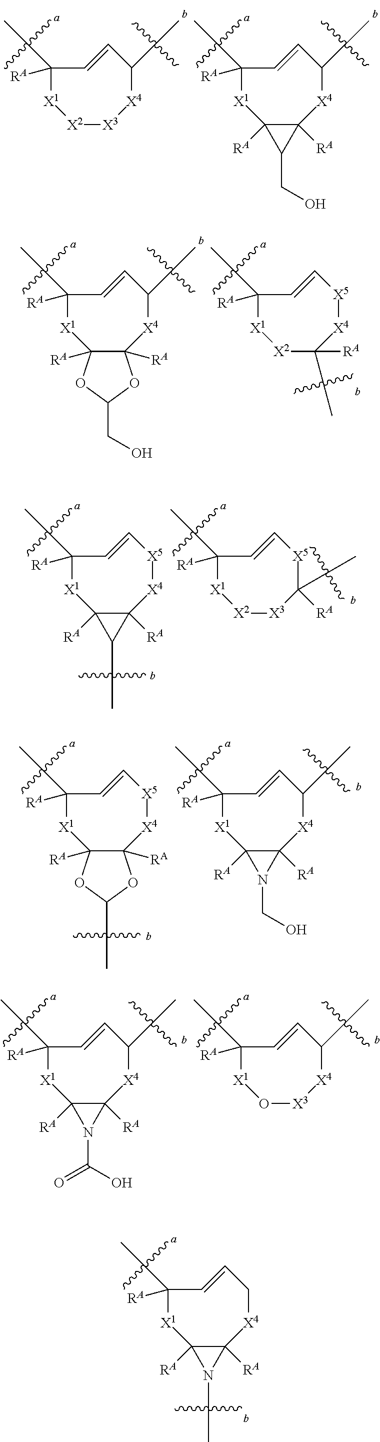

wherein a indicates a point of attachment to a moiety of formula O-(L¹)ₙ-Y¹, and b indicates a point of attachment to a moiety of formula (L²)ₙ-(Y²)ₒ—(Y³)ₚ.

In some embodiments, the compound of Formula (I) is selected from any one of the following formulae:

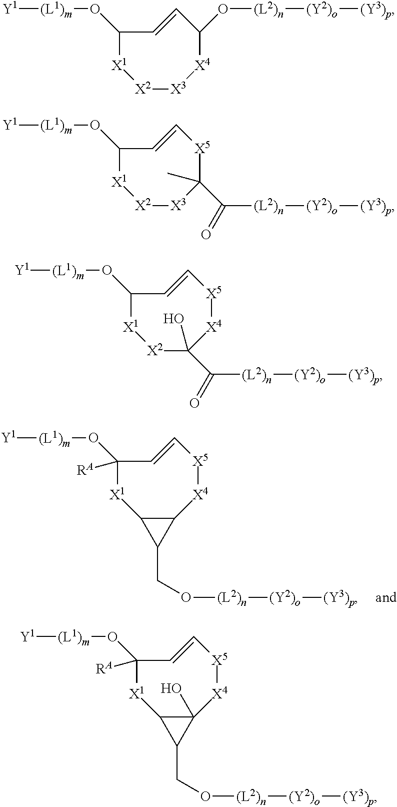

or a pharmaceutically acceptable salt thereof.

In some embodiments, the compound of Formula (I) has formula:

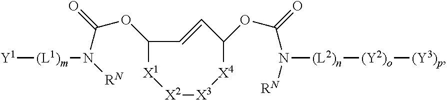

or a pharmaceutically acceptable salt thereof.

In some embodiments, the compound of Formula (I) is selected from any one of the following formulae:

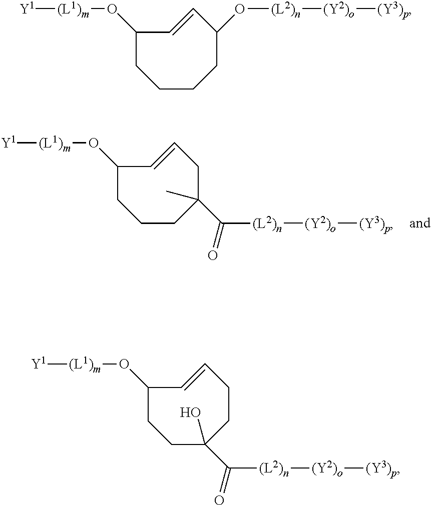

or a pharmaceutically acceptable salt thereof.

In some embodiments of Formula (I):
o is 1;
p is 1;
the chelating moiety is selected from the group consisting of 1,4,7-triazacyclononanetriacetic acid (NOTA), 1,4,7,10-tetraazacyclododecane-1,4,7,10-tetraacetic acid (DOTA), 1,4,7-triazacyclononane-1-glutaric acid-4,7-diacetic acid (NODAGA), ethylene diamine tetra-acetic acid (EDTA), diethylene triaminepentaacetic acid (DTPA), cyclohexyl-1,2-diaminetetraacetic acid (CDTA), ethyleneglycol-O,O′-bis(2-aminoethyl)-N,N,N′,N′-tetraacetic acid (EGTA), N,N-bis(hydroxybenzyl)-ethylenediamine-N,N′-diacetic acid (HBED), triethylene tetramine hexaacetic acid (TTHA), hydroxyethyidiamine triacetic acid (HEDTA), and 1,4,8,11-tetraazacyclotetradecane-N,N′,N″,N′″-tetraacetic acid (TETA), 1,4,7,10-tetraaza-1,4,7,10-tetra-(2-carbamoyl methyl)-cyclododecane (TCMC), and desferrioxamine B (DFO); and
the radiotracer is selected from ³H, ¹¹C, ¹⁴C, ¹⁸F, ³²P, ³⁵S, ³⁶Cl, ⁴⁴Sc, ⁴⁷Sc, ⁵¹Cr, ⁵²Fe, ^{52g}Mn, ⁵⁷Co, ⁵⁸Co, ⁵⁹Fe, ⁶⁴Cu, ⁶⁷Cu, ⁶⁷Ga, ⁶⁸Ga, ⁷⁵Se, ⁷⁶Br, ⁷⁷Br, ⁸²Rb, ⁸⁶Y, ⁸⁹Zr, ⁹⁰Y, ^{99m}Tc, ¹¹¹In, ^{114m}In, ¹²³I, ¹²⁴I, ¹²⁵I, ¹³¹I, ¹³³Xe, ¹⁵²Eu, ¹⁵³Sm, ¹⁶⁶Ho, ¹⁷⁷Lu, ¹⁸⁶Re, ¹⁸⁸Re, ²⁰¹Tl, ²⁰³Pb, ²¹⁰At, ²¹¹At, ²¹²Bi, ²¹²Pb, ²¹²Bi, ²¹³Bi, ²²³Ra, ²²⁵Ac, and ²²⁷Th.

In some embodiments, Y¹ is the affinity ligand is selected from an antibody, an antibody fragment, a fusion antibody, a chimeric antibody, a nanobody, an engineered antibody, a nanoparticle, an aptamers, an oligo, a peptide, a protein, or a small-molecule.

In some embodiments of Formula (I):
o is 1 and p is 0; and
Y² has any one of the following formulae:

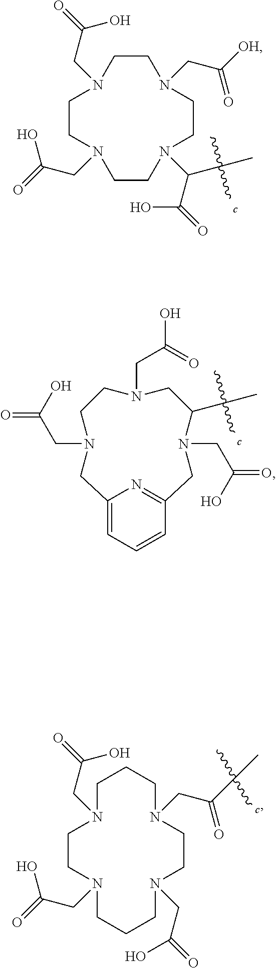 [SMILES: *C(C(=O)O)N1CCN(CC(=O)O)CCN(CC(=O)O)CCN(CC(=O)O)CC1 ; *C1CN(CC(=O)O)CCN(CC(=O)O)Cc2cccc(n2)CN1CC(=O)O ; *C(=O)CN1CCCN(CC(=O)O)CCN(CC(=O)O)CCCN(CC(=O)O)CC1]

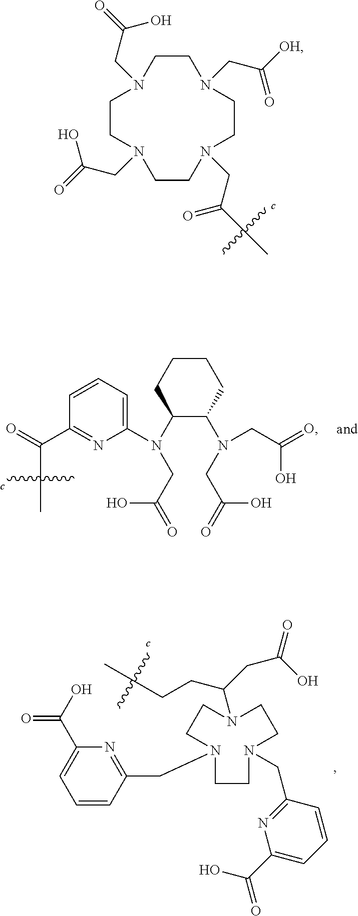 [SMILES: *C(=O)CN1CCN(CC(=O)O)CCN(CC(=O)O)CCN(CC(=O)O)CC1 ; *C(=O)c1cccc(N(CC(=O)O)[C@H]2CCCC[C@@H]2N(CC(=O)O)CC(=O)O)n1 ; *CCC(CC(=O)O)N1CCN(Cc2cccc(C(=O)O)n2)CCN(Cc2cccc(C(=O)O)n2)CC1]

wherein c indicates a point of attachment to L².

In some embodiments, the compound of Formula (I) has formula:

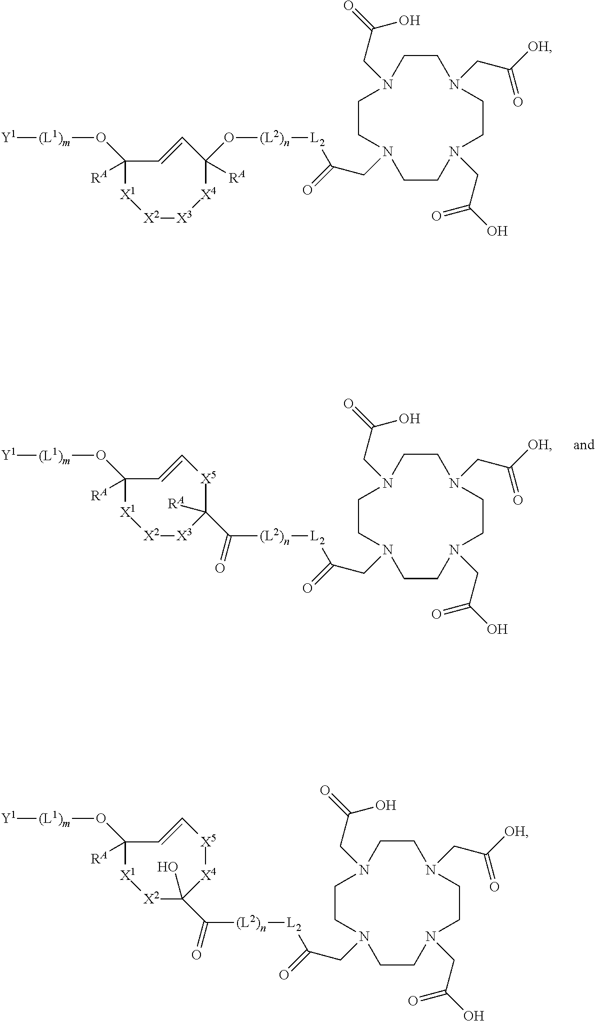

or a pharmaceutically acceptable salt thereof.

In some embodiments of Formula (I):
o is 1 and p is 1, and
the moiety Y²-Y³ has any one of the following formulae:

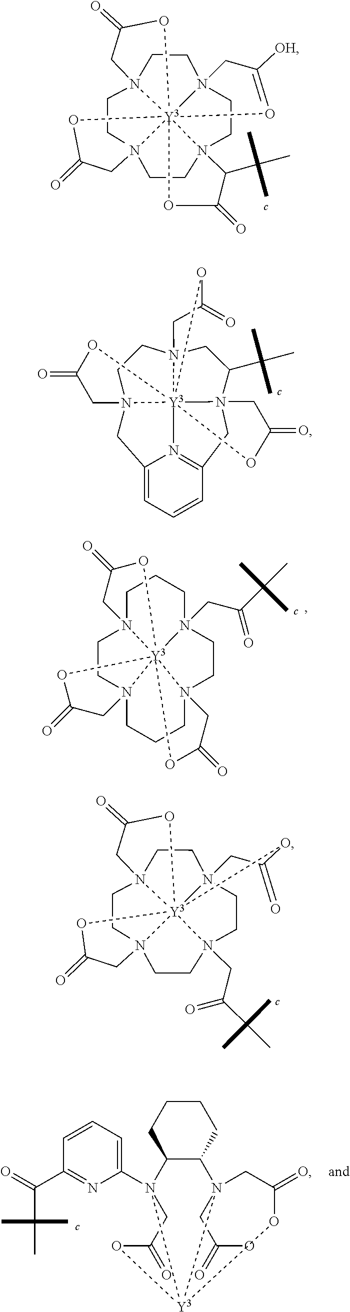

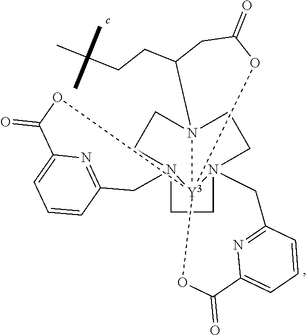

wherein c indicates a point of attachment to L².

In some embodiments, the compound of Formula (I) has any one of the following formulae (e.g., o is 1 and p is 1):

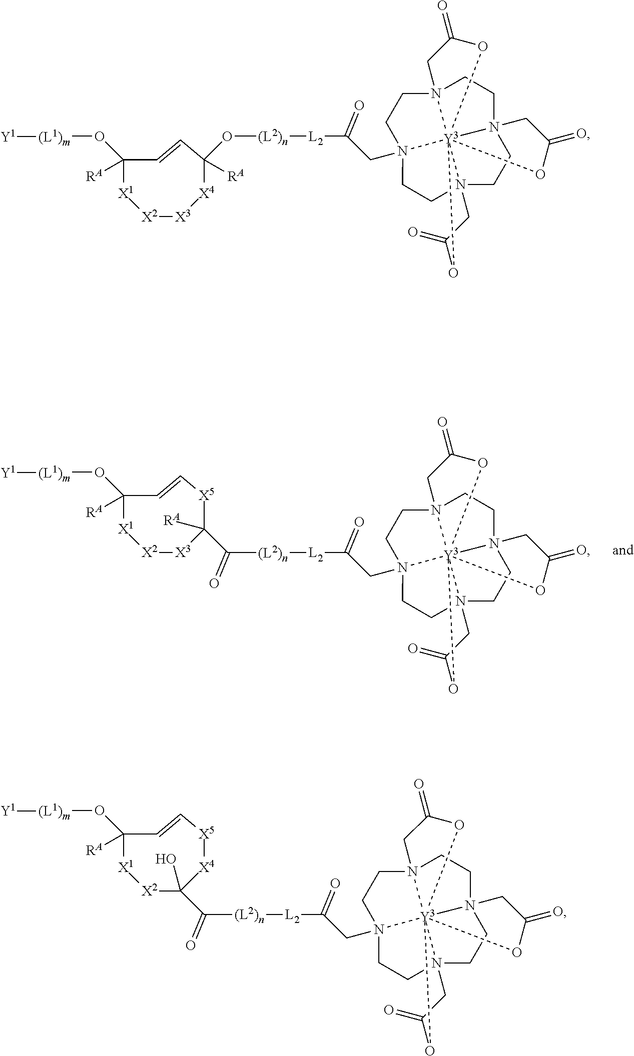

or a pharmaceutically acceptable salt thereof.

In some embodiments, the compound of Formula (I) is selected from any one of the following compounds (e.g., o is 1 and p is 1):

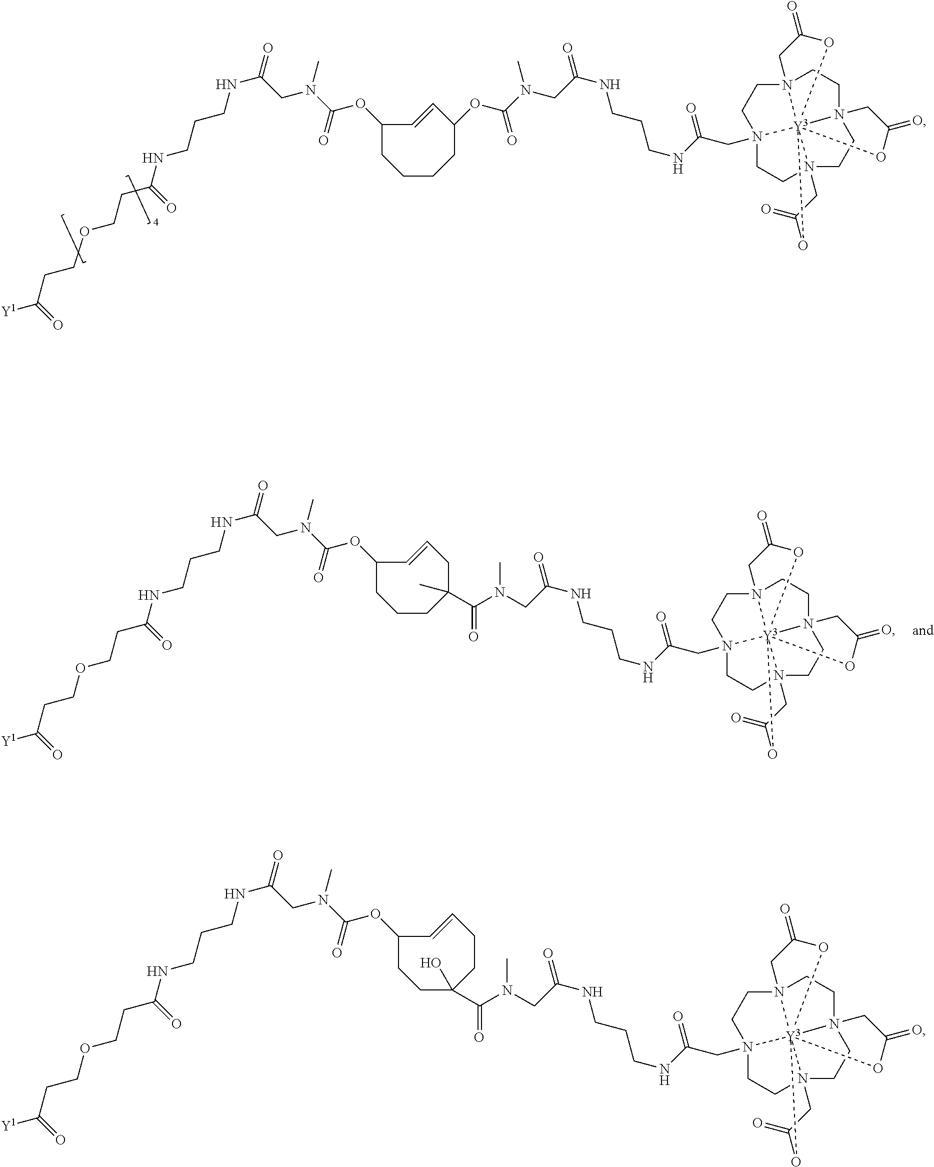

or a pharmaceutically acceptable salt thereof, wherein Y¹ is an affinity ligand.

In some embodiments, the compound of Formula (I) is selected from any one of the following compounds (e.g., o is 1 and p is 1):

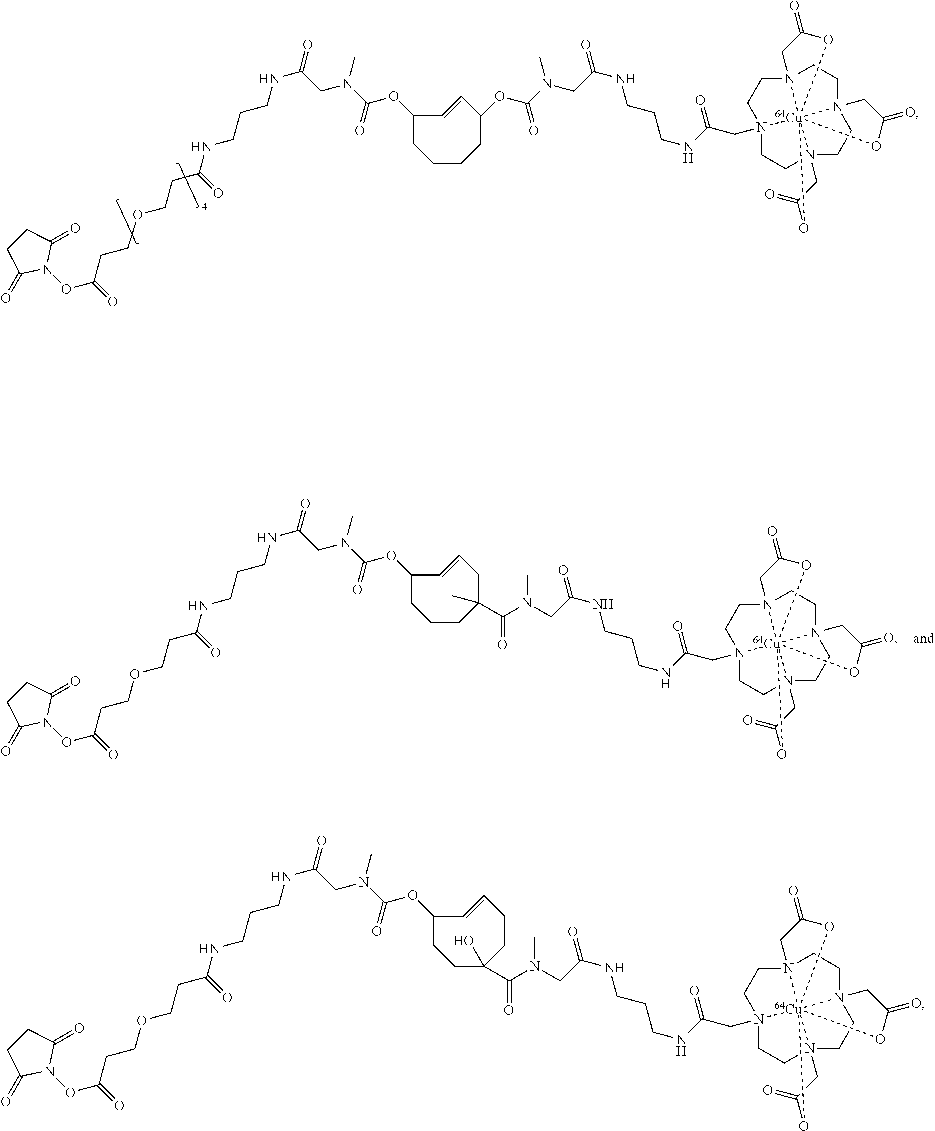

or a pharmaceutically acceptable salt thereof.

In some embodiments of Formula (I) is selected from any one of the following compounds (e.g., o is 1 and p is O):

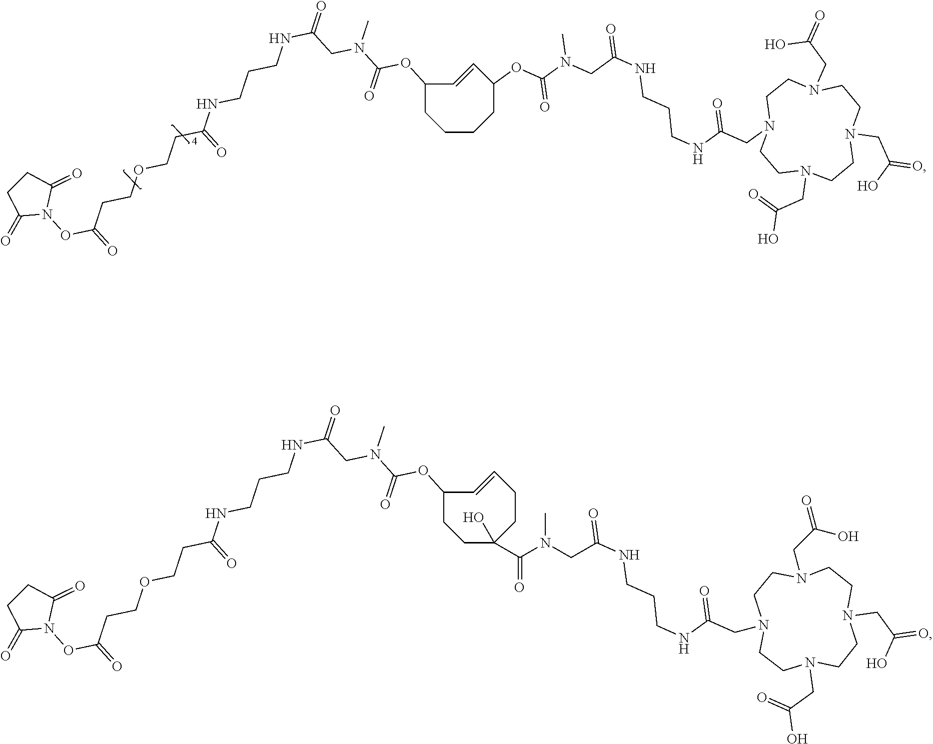

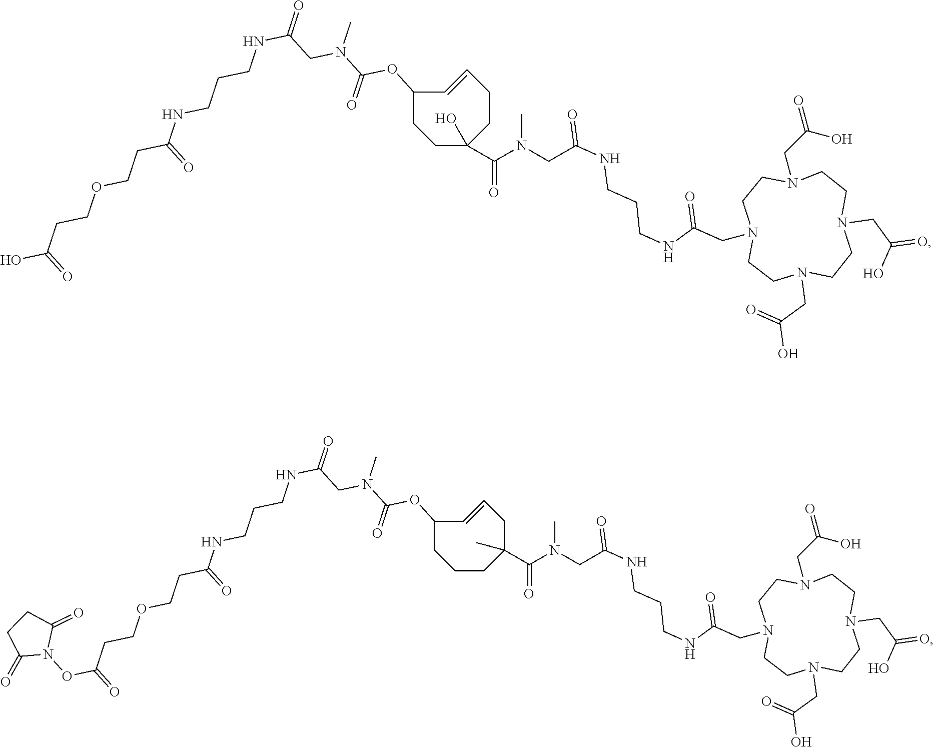 [SMILES: CN(CC(=O)NCCCNC(=O)CCOCCC(=O)O)C(=O)OC1/C=C/CCC(O)(C(=O)N(C)CC(=O)NCCCNC(=O)CN2CCN(CC(=O)O)CCN(CC(=O)O)CCN(CC(=O)O)CC2)CC1 ; CN(CC(=O)NCCCNC(=O)CCOCCC(=O)ON1C(=O)CCC1=O)C(=O)OC1/C=C/CC(C)(C(=O)N(C)CC(=O)NCCCNC(=O)CN2CCN(CC(=O)O)CCN(CC(=O)O)CCN(CC(=O)O)CC2)CCC1]

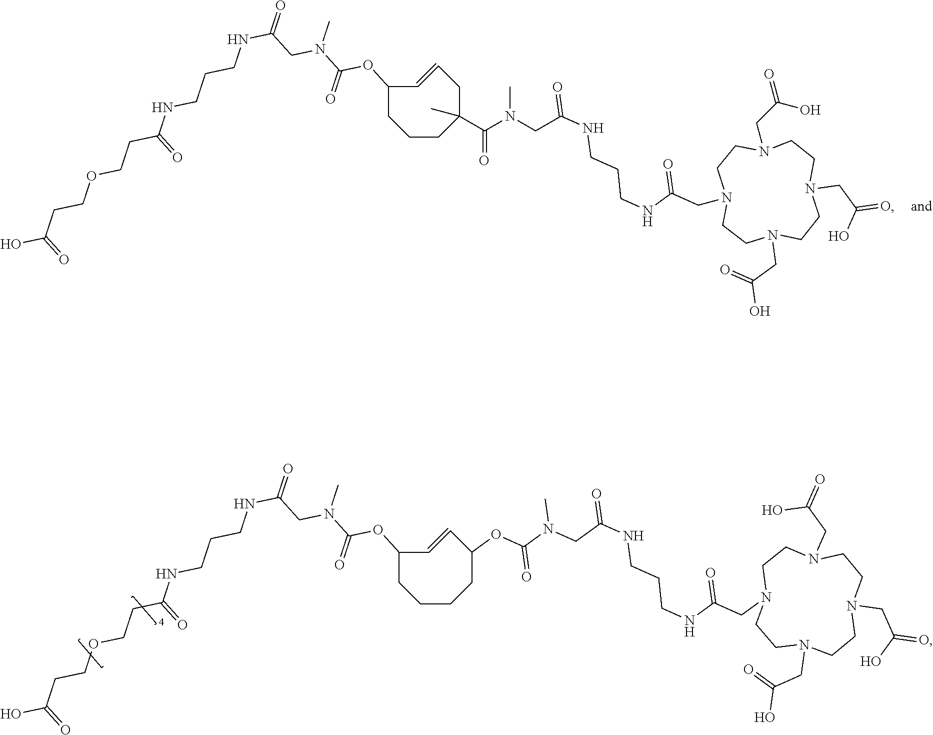

or a pharmaceutically acceptable salt thereof.

In another general aspect, the present disclosure provides a pharmaceutical composition comprising a compound of Formula (I) as described herein, or a pharmaceutically acceptable salt thereof, and a pharmaceutically acceptable carrier.

In yet another general aspect, the present disclosure provides a method of imaging a cell or tissue of a subject, the method comprising administering to the subject in need thereof a compound of Formula (I), or a pharmaceutically acceptable salt thereof, wherein
Y¹ is an affinity ligand; and
Y³ is a PET and/or imagable SPECT imagable radiotracer.

In some embodiments, the methods comprises a step of administering to the subject a compound of Formula (II):

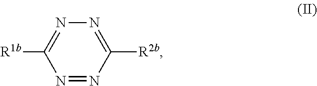

or a pharmaceutically acceptable salt thereof, wherein
R^{1b} and R^{2b} are each independently selected from H, C₁₋₆ alkyl, C₆₋₁₀ aryl, 5-6-membered heteroaryl, C₃₋₁₀ cycloalkyl, 4-7-membered heterocycloalkyl, wherein said C₁₋₆ alkyl, C₆₋₁₀ aryl, 5-6-membered heteroaryl, C₃₋₁₀ cycloalkyl, and 4-7-membered heterocycloalkyl are each optionally substituted with 1, 2, 3, or 4 substituents independently selected from R^{2c};
each R², is independently selected from halo, CN, NO₂, C₁₋₆ alkyl, C₁₋₆ haloalkyl, C₂₋₆ alkenyl, C₂₋₆ alkynyl, OR^{a1}, C(O)R^{b1}, C(O)NR^{c1}R^{d1}, C(O)OR^{a1}, OC(O)R^{b1}, OC(O)NR^{c1}R^{d1}NR^{c1}R^{d1}, NR^{c1}C(O)R^{b1}, NR^{c1}C(O)OR^{a1}; wherein said C₁₋₆ alkyl, C₂₋₆ alkenyl, and C₂₋₆ alkynyl are each optionally substituted with 1, 2 or 3 substituents independently selected from CN, NO₂, C₁₋₆ alkyl, C₁₋₆ haloalkyl, C₂₋₆ alkenyl, C₂₋₆ alkynyl, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1};
each R^{a1}, R^{b1}, R^{c1}, and R^{d1} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene are optionally substituted with 1, 2, 3, 4, or 5 substituents independently selected from R^{g}; and
each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In yet another general aspect, the present disclosure provides a method of treating a disease or condition in a subject, the method comprising administering to the subject in need thereof a therapeutically effective amount of a compound of Formula (I), or a pharmaceutically acceptable salt thereof, wherein:
Y¹ is an affinity ligand; and
Y³ is a toxic radiotracer.

In some embodiments, the toxic radiotracer is selected from ⁴⁷Sc, ⁹⁰Y, ^{114m}In, ¹³¹I, ¹⁷⁷Lu, ¹⁸⁶Re, ¹⁸⁸Re, ²¹¹At, ²¹²Bi, ²¹²Pb, ²¹²Bi, ²¹³Bi, ²²³Ra, ²²⁵Ac, and ^{227Th}.

In some embodiments, the method comprises a step of imaging the subject with an imaging technique selected from positron emission tomography (PET) imaging, positron emission tomography with computer tomography (PET/CT) imaging, positron emission tomography with magnetic resonance (PET/MRI) imaging, and single-photon emission computerized tomography (SPECT) imaging.

In some embodiments, the method comprises administering to the subject a compound of Formula (II) as described herein, or a pharmaceutically acceptable salt thereof.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. Other features and advantages of the present application will be apparent from the following detailed description and figures, and from the claims.

### DESCRIPTION OF DRAWINGS

FIG. 1A Affinity labels (antibodies, nanobodies, aptamers, proteins, peptides, oligo, nanoparticles) are labeled with a SENIT linker incorporating an immolative TCO linker such as C₂TCO. This stable construct is used for in vivo imaging and therapy. When a dose reduction is desired (long circulation time of affinity ligand), a second IV injection of Tz is performed and which cleaves the radiolabel-chelator complex in seconds. The latter is rapidly cleared by the kidneys resulting in dose reduction and favorable imaging/therapy kinetics.

FIG. 1B provides an example of radiation reduction and signal to nose ratio (SNR) increase through SENIT.

FIG. 1C provides examples of PET, SPECT and therapeutic payloads.

FIG. 2A provides an example of modeling of a SENIT dose reduction in PET imaging. In this modeling, a mouse is injected with anti-tumor antibody labeled with ⁶⁴Cu-SENIT antibody (left). Note the radioactivity is typically high in liver and the rest of body and does not contribute to diagnostic information. After IV injection of Tz-N, the radioactivity is rapidly cleared leading to i) dose reduction, ii) better tumor-background ratios and iii) the possibility of multiplexed PET imaging with another radio tracer (modeling experiment).

FIG. 2B contains line plots showing results of modeling of dose reduction and SNR following SENIT/Tz use in PET imaging.

FIG. 3A shows synthesis of SAFE647. The compound was synthesized as described previously, see Ko et al., 2022, Nat Biotechnol, 40, 1654-1662 (“Ko”) and U.S. application Ser. No. 18/247,571 (which are incorporated herein by reference in their entirety) via NHS amide coupling followed by activation of the free carboxylate using TSTU. The resulting NHS-ester was validated via LC-MS and used without further purification.

FIG. 3B shows synthesis of HK-Tz. The compound was prepared as previously described to yield the HCl salt, see Wilkovitsch et al., 2020, J Am Chem Soc, 142, 19132-19141 (“Wilkovitsch”) (which is incorporated herein by reference in its entirety).

FIG. 3C shows synthesis of SENIT tracer. The compound was prepared from a symmetrical bis-amino-C₂TCO (see Wilkovitsch), which was reacted sequentially with NHS-DOTA and bis-PEG₄-NHS under basic conditions to provide the desired compound.

FIG. 4 The sequential two-part reaction of SENIT-labeled antibodies is shown in (i). (ii) Time traces for the click and scission reactions. After collecting an initial baseline, BHQ3-N-Tz was added to the cuvette (magnetically stirred) for continuous monitoring of reaction progress. As the concentration of BHQ3-N-Tz is increased, the absolute rate of the click-quench reaction increases, leading to a deeper (and more rapid) nadir and ensuring accurate segregation of the two steps. Plotting the pseudo-first order rate constants vs BHQ3-N-Tz concentration yields a slope of 1.18×10⁶ M⁻¹s⁻¹ for the effective click rate. The fit data confirm that the scission rate is independent of concentration, as expected, and rapid, with a half-life of <5 seconds, corresponding in turn to a t_{99%} of ≤33 seconds.

FIG. 5 A431 cells were labeled with SENIT-anti-EGFR antibody. Note the rapid release of the imaging label upon addition of N-Tz. The signal was completely gone within about 20 seconds of addition.

FIG. 6A contains line plots showing LCMS analysis of DOTA-TCO-NHS with varying ratios of HK-Tz added in PBS (pH 7.4). DOTA-TCO-NHS was synthesized as described in FIG. 3C, Characterized by NMR and then subjected to Tz treatment. Aliquots of 100 μM DOTA linker were treated with 10 mM Tz-amine and analyzed by LCMS after 30 min and 3 hours to determine the percentage of intact linker remaining. Shown are the kinetics of linker immolation (T_{1/2} 12 min).

FIG. 6B contains a line plot showing stability of trastuzumab-TCO-DOTA loaded with ⁶⁴Cu over 72 hours at 37° C.

FIG. 7A contains a line plot showing percentage of dye released in vivo as a function of Tz dose. The percentage of dye released was determined by collecting the urine from the bladder of each mice 2 h after HK-Tz was administered (n=3). For each in vivo experiment, mice were treated via tail vein injection of trastuzumab-SAFE647 (1 nmol of dye), followed by injection of HK-Tz after 15 minutes.

FIG. 7B contains a line plot showing ear vasculature fluorescence following trastuzumab-SAFE647 and HK-Tz administration. Images were collected every 6 minutes as each reagent was administered, and the relative fluorescence intensity of the SAFE647 probe was quantified.

FIG. 8A schematically shows a proof of principle in vivo experiment. To determine the kinetics and release of the linker, fluorescently labeled HER2 and EGFR constructs were used. SENIT-antibody was injected via tail vein, followed at a later time by tail vein injection of the HK-Tz. Two hours later, the mice were sacrificed and the urine was collected directly from the bladder.

FIG. 8B contains an image showing that collected urine was found to be green and was successfully extracted from mice injected with SENIT-cetuximab (EGFR) and SENIT-trastuzumab (HER2).

FIG. 8C contains a bar graph showing percentage of dye recovered from nu/nu mice treated with SENIT antibody after HK-Tz administration.

### DETAILED DESCRIPTION

Conjugates useful in nuclear imaging technologies (such as PET and SPECT) and/or as radiotheranostics often include an antibody (or other affinity ligand) to impart tissue specificity connected (covalently or non-covalently) to a radionuclide (e.g., ⁶⁴Cu, ⁸⁹Zr). Some of these methods rely on long half-life affinity ligands (antibodies), which can lead to considerable radiotoxicity, including in non-target organs. This can limit (i) the dose of the conjugate that can be given, (ii) the number of imaging studies per year that can be performed (e.g., a limit of three PET scans per calendar year can make monitoring treatment challenging or even impossible), and (iii) the multiplexing capabilities of the technology (e.g., imaging organs and/or tissues affected by the disease using more than one affinity ligand or more than one radionuclide for a better diagnostic decision making or to more fully understand progression of treatment). Strategies previously utilized to avoid the foregoing limitations included reducing a dose of a radionuclide administered to the patient. This strategy is especially desirable for conjugates of long-circulating affinity ligands. Some forms of dose reduction are possible by whole body imaging (e.g., an emerging form of PET imaging with more detectors), renal protection programs (e.g., additional agents combined with theranostics to prevent renal toxicity), or pre-targeting (e.g., giving an antibody first followed by administration of a radionuclide attached to a moiety which recognizes the homed antibody via an affinity tag such as streptavidin-biotin.

Described herein are conjugates comprising a radionuclide (e.g., ⁶⁴Cu, ⁸⁹Zr, or ⁶⁸Ga) attached to an affinity ligand by a bioorthogonal “immolative” linker, such as a linker containing a trans-cyclooctene (C₂TCO) moiety. Certain embodiments of compositions including the conjugates, as well as methods of their use as imaging agents and/or as theranostic agents are also described. Without being bound by any theory or speculation, by administering a tetrazine (“Tz”)-containing antidote following administration of a radionuclide-labeled conjugate with a C₂TCO-containing linker, the methods within the present claims enable “scission”-enhanced cleavage of radioisotopes from the affinity ligand. Hence, the compounds and methods of this disclosure advantageously allow, inter alia, (i) reducing radiotoxicity by effectively reducing a dose of a toxic radioisotope due to its rapid corporeal elimination following the cleavage from a long half-life affinity ligand, (ii) performing nuclear multiplexed imaging using different affinity ligands and/or different radionuclides, and (iii) improving the target to background ratios.

### Compounds

In a general aspect, the present disclosure provides a compound (e.g., a conjugate) containing a releasable TCO moiety within its chemical structure (referred to herein as “TCO” or “rTCO”). In some embodiments, the present disclosure provides a compound of Formula (A):

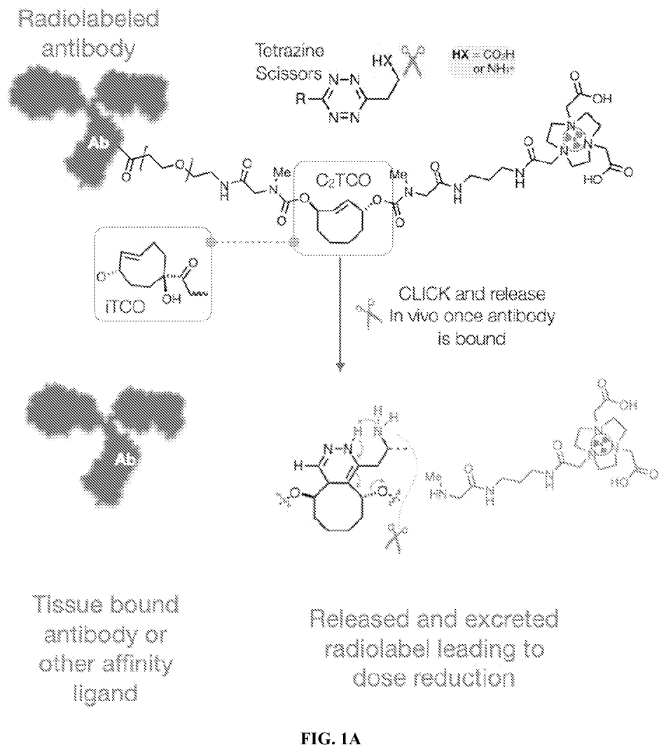

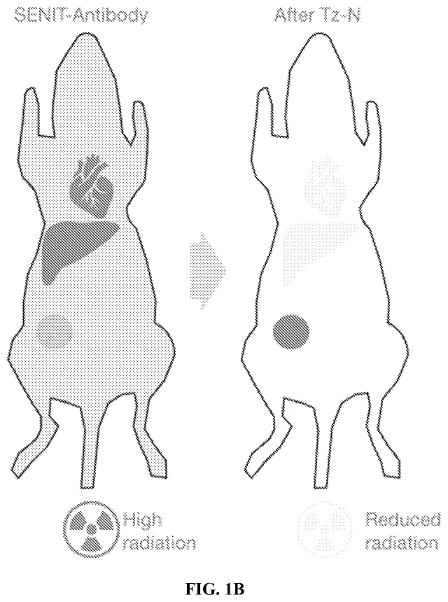

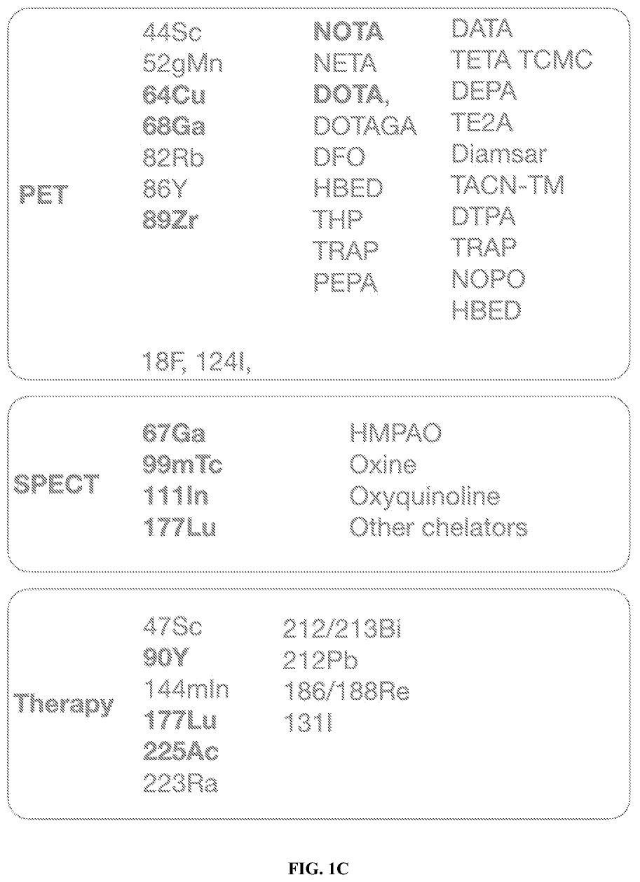

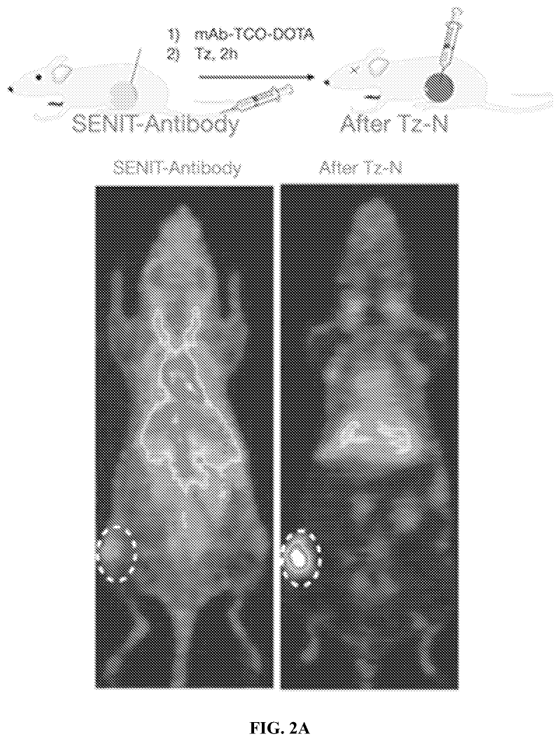

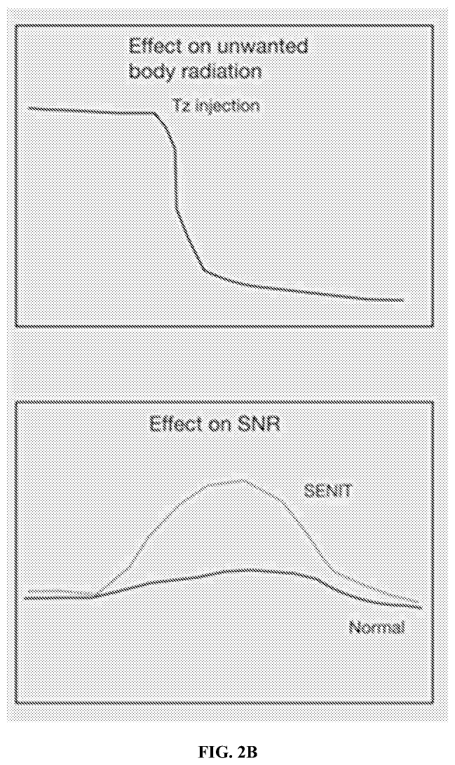

Y¹-L¹-TCO-L²-Y²-Y³ (A),
or a pharmaceutically acceptable salt thereof, wherein:
Y¹ is an affinity ligand (e.g., any of the affinity ligands as described herein, such as antibodies, antibody fragments, fusion antibodies, chimeric antibodies, nanobodies, nanoparticles, proteins, peptides, aptamers, oligo, small molecules, and other moieties and compounds that are useful for targeting cells, organs, and/or issues and as such are useful for nuclear imaging and as therapeutics) or a group reactive with the affinity ligand;
L¹ and L² are each independently a linker (e.g., comprising a plurality of functional groups as described herein, such as O, C(═O), NH₂, NHCH₃, C₁₋₆ alkylene, —CH₂CH₂O—, and/or —OCH₂CH₂—, among others); TCO is a trans-cyclooctene (e.g., a releasable trans-cyclooctene as described herein, such as rTCO, cTCO, iTCO, C₂TCO, sTCO, siTCO, sC₂TCO, dTCO, dC₂TCO, or aza-C₂TCO);
Y² is an optional chelator moiety (e.g., chelator moiety capable of forming a complex with a radionuclide, such as any of the chelators described herein, e.g., NOTA, DOTA, or DOTAGA, etc., see FIG. 1C for representative examples); and
Y³ is an optional radiotracer (e.g., any of the radioisotopes described herein, including those that are commonly used as clinical materials, see FIG. 1C for representative examples).

In some embodiments, the compound of Formula (A) comprises one or both of Y² and Y³ (e.g., if Y² is optional then Y³ is not optional; and if Y³ is optional then Y² is not optional). In one example, Y² may be omitted if Y³ can be directly attached to L² (e.g., if chelation is not necessary to incorporate a radioisotope into the chemical structure of the conjugate of Formula (A), for example, if the radioisotope is ¹⁸F, ¹¹C, or ^{123/124/125}I).

### Compounds of Formula (I)

In some embodiments, the compound of Formula (A) has Formula (I):

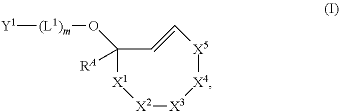

or a pharmaceutically acceptable salt thereof, wherein, X¹, X², X³, X⁴, X⁵, R^{A}, L¹, m, and Y¹ are as described herein (in any possible combination of described alternatives). In some embodiments:
X¹ is selected from O and CR^{A}R¹;
X² is selected from O and CR^{A}R²;
X³ is selected from O and CR^{A}R³;
X⁴ is selected from O and CR^{A}R⁴;
X⁵ is selected from O and CR^{A}R⁵;
each R^{A} is independently selected from H, OH, C₁₋₃ alkyl, and C₁₋₃ haloalkyl;
R¹, R², R³, R⁴, and R⁵ are each independently selected from H, halo, C₁₋₆ alkyl, C₁₋₆ haloalkyl, C₁₋₃ alkoxy, C₁₋₃ haloalkoxy, and a moiety of formula (L²)ₙ-(Y²)ₒ—(Y³)ₚ;
or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(═O)OH, C(═O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L²)ₙ-(Y²)ₒ—(Y³)ₚ;
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(═O)OH, C(═O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L²)ₙ-(Y²)ₒ—(Y³)ₚ;
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(═O)OH, C(═O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L²)ₙ-(Y²)ₒ—(Y³)ₚ;
or R⁴ and R⁵, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(═O)OH, C(═O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L²)ₙ-(Y²)ₒ—(Y³)ₚ;
each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(═O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
each L² is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(═O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
each x is independently an integer from 1 to 2,000;
each R^{N} is independently selected from H, C₁₋₃ alkyl, and C₁₋₃ haloalkyl;
Y¹ is selected from NH₂, OH, C(═O)OH, a protected amino group, a protected hydroxyl group, a protected carboxyl group, a reactive chemical group (e.g., a chemical group reactive with an affinity ligand), and an affinity ligand;
m is an integer from 1 to 20;
n is an integer from 1 to 20;
o is 0, 1, 2, or 3;
p is 0, 1, 2, or 3;
Y² is a chelating moiety; and
Y³ is a radiotracer.

In some embodiments, at least one of o and p is 1. In some embodiments, o is 1. In some embodiments, p is 1. In some embodiments, o is 1 and p is 1. In some embodiments, o is 0 and p is 1. In some embodiments, o is 1 and p is 0. In some embodiments, o is 2 and p is 2.

In some embodiments, o is 0. In some embodiments, o is 2. In some embodiments, o is 3. In some embodiments, p is 0. In some embodiments, p is 2. In some embodiments, p is 3.

### TCO Moieties

In some embodiments, R^{A} is H. In some embodiments, R^{A} is OH. In some embodiments, R^{A} is C₁₋₃ alkyl (e.g., optionally substituted with OH, NH₂, C(═O)OH, C(═O)C₁₋₃ alkoxy, C₁₋₃ alkoxy, or C₁₋₃ haloalkoxy). In some embodiments, X¹ is O. In some embodiments, X¹ is CHR¹. In some embodiments, X² is O. In some embodiments, X² is CHR². In some embodiments, X³ is O. In some embodiments, X³ is CHR³. In some embodiments, X⁴ is O. In some embodiments, X⁴ is CHR⁴. In some embodiments, X⁵ is O. In some embodiments, X⁵ is CHR⁵. In some embodiments, X¹ is CH₂. In some embodiments, X² is CH₂. In some embodiments, X³ is CH₂. In some embodiments, X⁴ is CH₂. In some embodiments, X⁵ is CH₂. In some embodiments, no more than 1 or 2 of X¹, X², X³, X⁴, and X⁵ are O. In some embodiments, no more than 1 of X¹, X², X³, X⁴, and X⁵ is O. In some embodiments, only 1 of X¹, X², X³, X⁴, and X⁵ is O. In some embodiments, X¹ is CHR¹, X² is CHR², X³ is CHR³, X⁴ is CHR⁴, and X⁵ is CHR⁵.

In some embodiments, R¹ is selected from H, halo, C₁₋₃ alkyl, C₁₋₃ haloalkyl, C₁₋₃ alkoxy, C₁₋₃ haloalkoxy, and a moiety of formula (L²)ₙ-(Y²)ₒ—(Y³)ₚ. In some embodiments, R¹ is H. In some embodiments, R¹ is selected from H, halo, and C₁₋₃ alkyl. In some embodiments, R¹ is (L²)ₙ-(Y²)ₒ—(Y³)ₚ. In some embodiments, R¹ is —O-(L²)ₙ-(Y²)ₒ—(Y³)ₚ (e.g., L² group attached to CR^{A} is O).

In some embodiments, R² is selected from H, halo, C₁₋₃ alkyl, C₁₋₃ haloalkyl, C₁₋₃ alkoxy, C₁₋₃ haloalkoxy, and a moiety of formula (L²)ₙ-(Y²)ₒ—(Y³)ₚ. In some embodiments, R² is H. In some embodiments, R² is selected from H, halo, and C₁₋₃ alkyl. In some embodiments, R² is (L²)ₙ-(Y²)ₒ—(Y³)ₚ. In some embodiments, R² is —O-(L²)ₙ-(Y²)ₒ—(Y³)ₚ (e.g., L² group attached to CR^{A} is O).

In some embodiments, R³ is selected from H, halo, C₁₋₃ alkyl, C₁₋₃ haloalkyl, C₁₋₃ alkoxy, C₁₋₃ haloalkoxy, and a moiety of formula (L²)ₙ-(Y²)ₒ—(Y³)ₚ. In some embodiments, R³ is H. In some embodiments, R³ is selected from H, halo, and C₁₋₃ alkyl. In some embodiments, R³ is (L²)ₙ-(Y²)ₒ—(Y³)ₚ. In some embodiments, R³ is —O-(L²)ₙ-(Y²)ₒ—(Y³)ₚ (e.g., L² group attached to CR^{A} is O).

In some embodiments, R⁴ is selected from H, halo, C₁₋₃ alkyl, C₁₋₃ haloalkyl, C₁₋₃ alkoxy, C₁₋₃ haloalkoxy, and a moiety of formula (L²)ₙ-(Y²)ₒ—(Y³)ₚ. In some embodiments, R⁴ is H. In some embodiments, R⁴ is selected from H, halo, and C₁₋₃ alkyl. In some embodiments, R⁴ is (L²)ₙ-(Y²)ₒ—(Y³)ₚ. In some embodiments, R⁴ is —O-(L²)ₙ-(Y²)ₒ—(Y³)ₚ (e.g., L² group attached to CR^{A} is O).

In some embodiments, R⁵ is selected from H, halo, C₁₋₃ alkyl, C₁₋₃ haloalkyl, C₁₋₃ alkoxy, C₁₋₃ haloalkoxy, and a moiety of formula (L²)ₙ-(Y²)ₒ—(Y³)ₚ. In some embodiments, R⁵ is H. In some embodiments, R⁵ is selected from H, halo, and C₁₋₃ alkyl. In some embodiments, R⁵ is (L²)ₙ-(Y²)ₒ—(Y³)ₚ. In some embodiments, R⁵ is —O-(L²)ₙ-(Y²)ₒ—(Y³)ₚ (e.g., L² group attached to CR^{A} is O).

In some embodiments, R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(═O)OH, C(═O)C₁₋₃ alkoxy, C₁₋₃ alkyl, HO—C₁₋₃ alkylene, and H₂N—C₁₋₃ alkylene. In some embodiments, any ring formed by R¹ and R² as described herein is optionally substituted with (L²)ₙ-(Y²)ₒ—(Y³)ₚ. In some embodiments, any ring formed by R¹ and R² as described herein is optionally substituted with —O-(L²)ₙ-(Y²)ₒ—(Y³)ₚ (e.g., L² group attached to CR^{A} is O).

In some embodiments, R¹ and R², together with the carbon atoms to which they are attached, form an C₆₋₁₀ aryl ring (e.g., phenyl).

In some embodiments, R¹ and R², together with the carbon atoms to which they are attached, form a C₃₋₁₀ cycloalkyl ring (e.g., cyclopropyl, cyclobutyl, cyclopentyl, or cyclohexyl), which is optionally substituted with 1 or 2 substituents independently selected from C(═O)OH, C(═O)C₁₋₃ alkoxy, C₁₋₃ alkyl, HO—C₁₋₃ alkylene, and H₂N—C₁₋₃ alkylene.

In some embodiments, R¹ and R², together with the carbon atoms to which they are attached, form a 5-14 membered heteroaryl ring (e.g., pyridinyl), which is optionally substituted with 1 or 2 substituents independently selected from C(═O)OH, C(═O)C₁₋₃ alkoxy, C₁₋₃ alkyl, HO—C₁₋₃ alkylene, and H₂N—C₁₋₃ alkylene.

In some embodiments, R¹ and R², together with the carbon atoms to which they are attached, form a 4-10 membered heterocycloalkyl ring (e.g., azetidine, aziridine, 1,3-dioxolane, oxazolidin-2-one, or oxetane), which is optionally substituted with 1 or 2 substituents independently selected from C(═O)OH, C(═O)C₁₋₃ alkoxy, C₁₋₃ alkyl, HO—C₁₋₃ alkylene, and H₂N—C₁₋₃ alkylene.

In some embodiments, R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(═O)OH, C(═O)C₁₋₃ alkoxy, C₁₋₃ alkyl, HO—C₁₋₃ alkylene, and H₂N—C₁₋₃ alkylene. In some embodiments, any ring formed by R² and R³ as described herein is optionally substituted with (L²)ₙ-(Y²)ₒ—(Y³)ₚ. In some embodiments, any ring formed by R² and R³ as described herein is optionally substituted with —O-(L²)ₙ-(Y²)ₒ—(Y³)ₚ (e.g., L² group attached to CR^{A} is O).

In some embodiments, R² and R³, together with the carbon atoms to which they are attached, form an C₆₋₁₀ aryl ring (e.g., phenyl).

In some embodiments, R² and R³, together with the carbon atoms to which they are attached, form a C₃₋₁₀ cycloalkyl ring (e.g., cyclopropyl, cyclobutyl, cyclopentyl, or cyclohexyl), which is optionally substituted with 1 or 2 substituents independently selected from C(═O)OH, C(═O)C₁₋₃ alkoxy, C₁₋₃ alkyl, HO—C₁₋₃ alkylene, and H₂N—C₁₋₃ alkylene.

In some embodiments, R² and R³, together with the carbon atoms to which they are attached, form a 5-14 membered heteroaryl ring (e.g., pyridinyl), which is optionally substituted with 1 or 2 substituents independently selected from C(═O)OH, C(═O)C₁₋₃ alkoxy, C₁₋₃ alkyl, HO—C₁₋₃ alkylene, and H₂N—C₁₋₃ alkylene.

In some embodiments, R² and R³, together with the carbon atoms to which they are attached, form a 4-10 membered heterocycloalkyl ring (e.g., azetidine, aziridine, 1,3-dioxolane, oxazolidin-2-one, or oxetane), which is optionally substituted with 1 or 2 substituents independently selected from C(═O)OH, C(═O)C₁₋₃ alkoxy, C₁₋₃ alkyl, HO—C₁₋₃ alkylene, and NH₂—C₁₋₃ alkylene.

In some embodiments, R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(═O)OH, C(═O)C₁₋₃ alkoxy, C₁₋₃ alkyl, HO—C₁₋₃ alkylene, and H₂N—C₁₋₃ alkylene. In some embodiments, any ring formed by R³ and R⁴ as described herein is optionally substituted with (L²)ₙ-(Y²)ₒ—(Y³)ₚ. In some embodiments, any ring formed by R³ and R⁴ as described herein is optionally substituted with —O-(L²)ₙ-(Y²)ₒ—(Y³)ₚ (e.g., L² group attached to CR^{A} is O).

In some embodiments, R³ and R⁴, together with the carbon atoms to which they are attached, form an C₆₋₁₀ aryl ring (e.g., phenyl).

In some embodiments, R³ and R⁴, together with the carbon atoms to which they are attached, form a C₃₋₁₀ cycloalkyl ring (e.g., cyclopropyl, cyclobutyl, cyclopentyl, or cyclohexyl), which is optionally substituted with 1 or 2 substituents independently selected from C(═O)OH, C(═O)C₁₋₃ alkoxy, C₁₋₃ alkyl, HO—C₁₋₃ alkylene, and H₂N—C₁₋₃ alkylene.

In some embodiments, R³ and R⁴, together with the carbon atoms to which they are attached, form a 5-14 membered heteroaryl ring (e.g., pyridinyl), which is optionally substituted with 1 or 2 substituents independently selected from C(═O)OH, C(═O)C₁₋₃ alkoxy, C₁₋₃ alkyl, HO—C₁₋₃ alkylene, and H₂N—C₁₋₃ alkylene.

In some embodiments, R³ and R⁴, together with the carbon atoms to which they are attached, form a 4-10 membered heterocycloalkyl ring (e.g., azetidine, aziridine, 1,3-dioxolane, oxazolidin-2-one, or oxetane), which is optionally substituted with 1 or 2 substituents independently selected from C(═O)OH, C(═O)C₁₋₃ alkoxy, C₁₋₃ alkyl, HO—C₁₋₃ alkylene, and H₂N—C₁₋₃ alkylene.

In some embodiments, R⁴ and R⁵, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(═O)OH, C(═O)C₁₋₃ alkoxy, C₁₋₃ alkyl, HO—C₁₋₃ alkylene, and H₂N—C₁₋₃ alkylene. In some embodiments, any ring formed by R⁴ and R⁵ as described herein is optionally substituted with (L²)ₙ-(Y²)ₒ—(Y³)ₚ. In some embodiments, any ring formed by R⁴ and R⁵ as described herein is optionally substituted with —O-(L²)ₙ-(Y²)ₒ—(Y³)ₚ (e.g., L² group attached to CR^{A} is O).

In some embodiments, R⁴ and R⁵, together with the carbon atoms to which they are attached, form an C₆₋₁₀ aryl ring (e.g., phenyl).

In some embodiments, R⁴ and R⁵, together with the carbon atoms to which they are attached, form a C₃₋₁₀ cycloalkyl ring (e.g., cyclopropyl, cyclobutyl, cyclopentyl, or cyclohexyl), which is optionally substituted with 1 or 2 substituents independently selected from C(═O)OH, C(═O)C₁₋₃ alkoxy, C₁₋₃ alkyl, HO—C₁₋₃ alkylene, and H₂N—C₁₋₃ alkylene.

In some embodiments, R⁴ and R⁵, together with the carbon atoms to which they are attached, form a 5-14 membered heteroaryl ring (e.g., pyridinyl), which is optionally substituted with 1 or 2 substituents independently selected from C(═O)OH, C(═O)C₁₋₃ alkoxy, C₁₋₃ alkyl, HO—C₁₋₃ alkylene, and H₂N—C₁₋₃ alkylene.

In some embodiments, R⁴ and R⁵, together with the carbon atoms to which they are attached, form a 4-10 membered heterocycloalkyl ring (e.g., azetidine, aziridine, 1,3-dioxolane, oxazolidin-2-one, or oxetane), which is optionally substituted with 1 or 2 substituents independently selected from C(═O)OH, C(═O)C₁₋₃ alkoxy, C₁₋₃ alkyl, HO—C₁₋₃ alkylene, and H₂N—C₁₋₃ alkylene.

In some embodiments, the TCO fragment within Formula (I) is selected from any one of the following moieties, wherein a indicates a point of attachment to a moiety of formula O-(L¹)ₙ-Y¹, and b indicates a point of attachment to a moiety of formula (L²)ₙ-(Y²)ₙ—(Y³)ₚ (e.g., to a moiety of formula 0-(L²)ₙ-(Y²)ₒ—(Y³)ₚ):

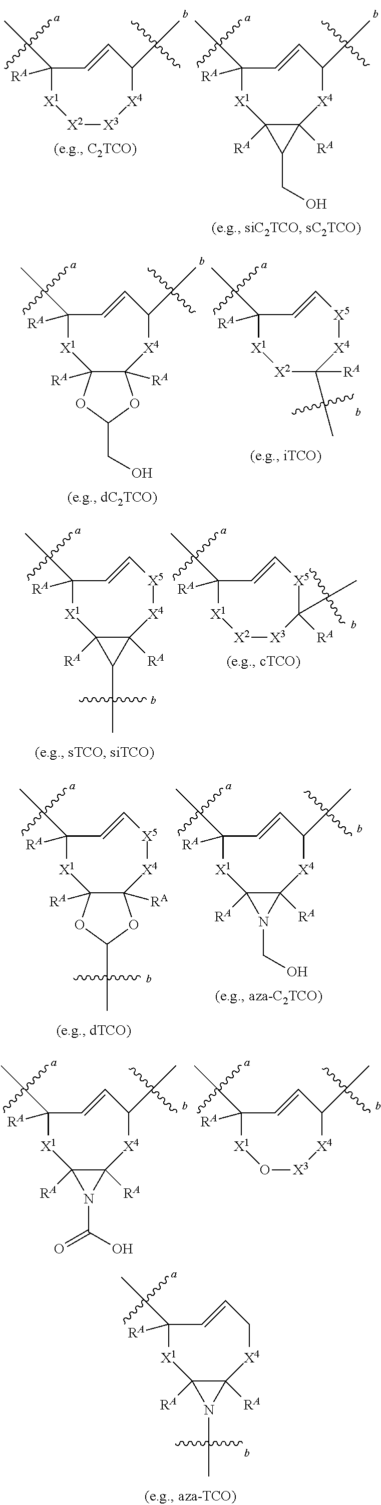

Additional suitable examples of TCO linkers useful in the compounds of Formula (I) can be found in Wilkovitsch and in WO/2022/072949, which are incorporated herein by references in their entirety.

Linker Moieties Separating (i) Affinity Ligand and TCO Moiety (Linker (L¹)ₘ), and (ii) TCO Moiety and the Radiotracer (Linker (L²)ₙ)

In some embodiments, each L¹ is independently selected from N(R^{N}), O, C(═O), C₁₋₆ alkylene, —(OCH₂CH₂)ₓ—, —(CH₂CH₂O)ₓ—, an amino acid, a self-immolative group, and a moiety formed by a click reaction, each of which is optionally substituted with 1 or 2 substituents independently selected from C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy. In some embodiments, each L¹ is independently selected from N(R^{N}), O, C(═O), C₁₋₆ alkylene, C₃₋₇ cycloalkylene, C₆₋₁₀ arylene, —(OCH₂CH₂)ₓ—, and —(CH₂CH₂O)ₓ—. In some embodiments, each L¹ is independently selected from 4-10-membered heterocycloalkylene and 5-10-membered heteroarylene, each of which is optionally substituted with 1 or 2 substituents independently selected from C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy. In some embodiments, each L¹ is independently selected from N(R^{N}), O, C(═O), C₁₋₆ alkylene, —(OCH₂CH₂)ₓ—, and —(CH₂CH₂O)ₓ—.

In some embodiments, at least one L¹ is N(R^{N}). In some embodiments, at least one L¹ is NH. In some embodiments, at least one L¹ is NCH₃. In some embodiments, at least one L¹ is O. In some embodiments, at least one L¹ is C(═O). In some embodiments, at least one L¹ is C₁₋₆ alkylene, optionally substituted with C(═O)OH, SO₃H, C₁₋₃ alkylamino, di(C₁₋₃-alkyl)amino, C₁₋₃ haloalkyl, C₁₋₃ alkoxy, and C₁₋₃ haloalkoxy. In some embodiments, at least one L¹ is an amino acid. In some embodiments, at least one L¹ is a self-immolative group. In some embodiments, at least one L¹ is a moiety formed by a click reaction.

In some embodiments, group (L¹)ₘ comprises at least one 4-10-membered heterocycloalkylene. In some embodiments, group (L¹)ₘ comprises at least one C₆₋₁₀ arylene. In some embodiments, group (L¹)ₘ comprises at least one moiety C(═O)O, OC(═O), C(═O)NH, C(═O)NCH₃, NHC(═O)NH, NHC(═S)NH, OC(═O)NH, or NHC(═O)O. In some embodiments, at least one L¹ is —(OCH₂CH₂)ₓ— or —(CH₂CH₂O)ₓ—. In some embodiments, group (L¹)ₘ comprises at least one C(═O)NHCH₂CH₂CH₂NHC(═O). In some embodiments, group (L¹)ₘ comprises at least one CH₂C(═O)NH.

In some embodiments, the (L¹)ₘ moiety comprises any one of the foregoing flexible structural fragments, or any combination thereof:

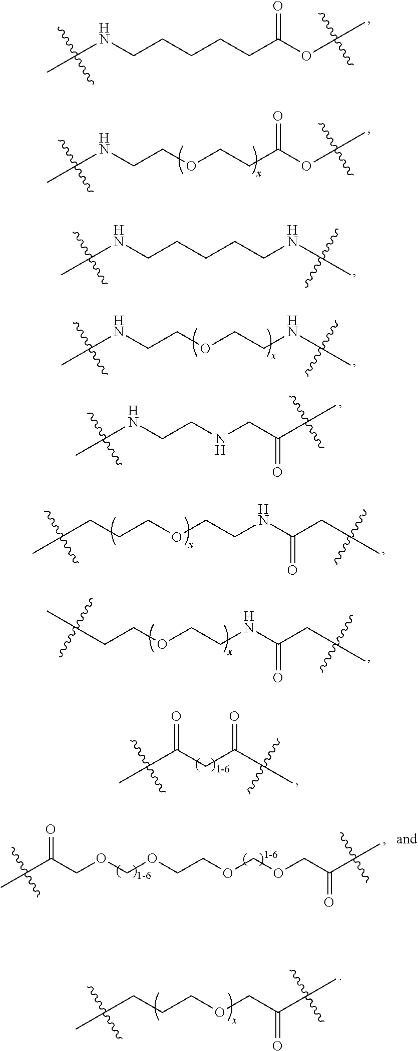

In some embodiments, the (L¹)ₘ moiety comprises any one of the foregoing rigid structural fragments, or any combination thereof:

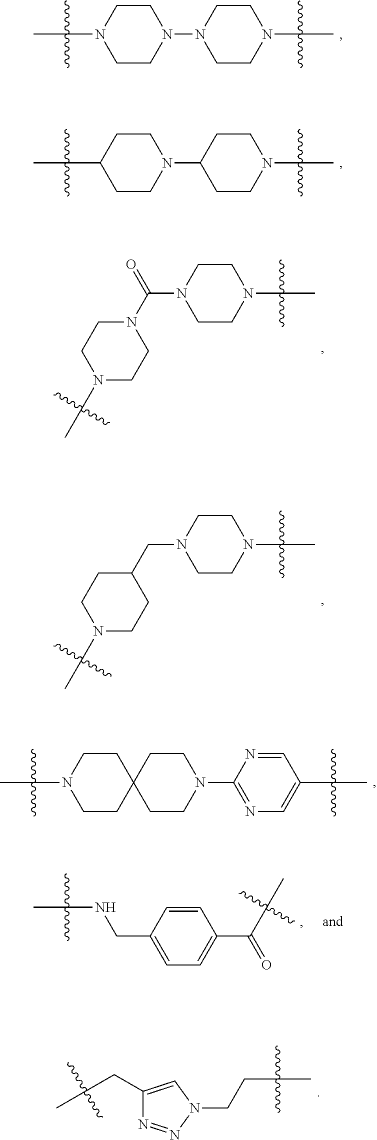

In some embodiments, m is an integer from 1 to 20. In some embodiments, m is an integer from 2 to 10. In some embodiments, m is 1, 2, 3, 4, or 5. In some embodiments, m is 1, 2, 3, 4, 5, 6, 7, 8, 9, or 10. In some embodiments, m is 0.

In some embodiments, each L² is independently selected from N(R^{N}), O, C(═O), C₁₋₆ alkylene, —(OCH₂CH₂)ₓ—, —(CH₂CH₂O)ₓ—, an amino acid, a self-immolative group, and a moiety formed by a click reaction, each of which is optionally substituted with 1 or 2 substituents independently selected from C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy. In some embodiments, each L² is independently selected from N(R^{N}), O, C(═O), C₁₋₆ alkylene, C₃₋₇ cycloalkylene, C₆₋₁₀ arylene, —(OCH₂CH₂)ₓ—, and —(CH₂CH₂O)ₓ—. In some embodiments, each L² is independently selected from 4-10-membered heterocycloalkylene and 5-10-membered heteroarylene, each of which is optionally substituted with 1 or 2 substituents independently selected from C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy. In some embodiments, each L² is independently selected from N(R^{N}), O, C(═O), C₁₋₆ alkylene, —(OCH₂CH₂)ₓ—, and —(CH₂CH₂O)ₓ—.

In some embodiments, at least one L² is N(R^{N}). In some embodiments, at least one L² is NH. In some embodiments, at least one L² is NCH₃. In some embodiments, at least one L² is 0. In some embodiments, at least one L² is C(═O). In some embodiments, at least one L² is C₁₋₆ alkylene, optionally substituted with C(═O)OH, SO₃H, C₁₋₃ alkylamino, di(C₁₋₃-alkyl)amino, C₁₋₃ haloalkyl, C₁₋₃ alkoxy, and C₁₋₃ haloalkoxy. In some embodiments, at least one L² is an amino acid. In some embodiments, at least one L² is a self-immolative group. In some embodiments, at least one L² is a moiety formed by a click reaction.

In some embodiments, group (L²)ₙ comprises at least one 4-10-membered heterocycloalkylene. In some embodiments, group (L²)ₙ comprises at least one C₆₋₁₀ arylene. In some embodiments, group (L²)ₙ comprises at least one moiety C(═O)O, OC(═O), C(═O)NH, C(═O)NCH₃, NHC(═O)NH, NHC(═S)NH, OC(═O)NH, or NHC(═O)O. In some embodiments, at least one L² is —(OCH₂CH₂)ₓ— or —(CH₂CH₂O)ₓ—. In some embodiments, group (L²)ₙ comprises at least one C(═O)NHCH₂CH₂CH₂NHC(═O). In some embodiments, group (L²)ₙ comprises at least one CH₂C(═O)NH.

In some embodiments, the (L²)ₙ moiety comprises any one of the foregoing flexible structural fragments, or any combination thereof:

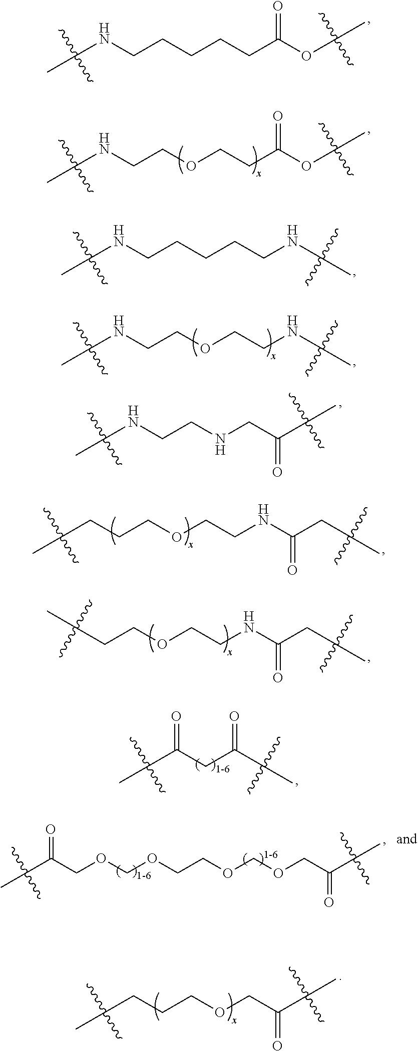

In some embodiments, the (L²)ₙ moiety comprises any one of the foregoing rigid structural fragments, or any combination thereof:

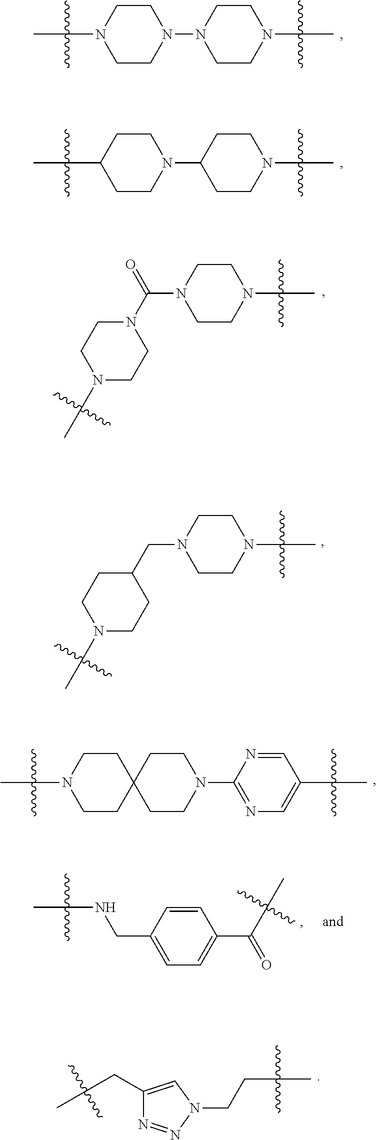

In some embodiments, n is an integer from 1 to 20. In some embodiments, n is an integer from 2 to 10. In some embodiments, n is 1, 2, 3, 4, or 5. In some embodiments, n is 1, 2, 3, 4, 5, 6, 7, 8, 9, or 10. In some embodiments, n is 0.

In some embodiments, each x is independently an integer from 1 to 2,000 (e.g., 10, 20, 100, 200, or 500).

In some embodiments, each R^{N} is independently selected from H, C₁₋₃ alkyl, and C₁₋₃ haloalkyl. In some embodiments, R^{N} is H. In some embodiments, R^{N} is C₁₋₃ alkyl.

### Amino Acids and Amino Acid Linker Moieties

Suitable examples of amino acids that could be used in L¹ or L² include lysine, arginine, histidine, aspartic acid, glutamic acid, serine, threonine, asparagine, glutamine, cysteine, glycine, proline, alanine, valine, isoleucine, leucine, methionine, phenylalanine, tyrosine, and tryptophan. Compounds and linker moieties with protected side chain functional groups are also encompassed by the term “amino acid” as used herein. A linker may include any n number of amino acids, for example, the linker can include (Gly)ₙ, where Gly can be N-linked or O-linked to any part of the remainder of the linker, and n can be an integer from 1 to 100 (e.g., 2, 3, 4, 5, 6, 7, 8, 9, or 10).

As used herein, the term “amino acid” generally refers to organic compounds containing amine (—NH₂) and carboxyl (—COOH) functional groups, along with a side chain (R group) specific to each amino acid. The side chain may be hydrophobic or hydrophilic, charged or neutral, as well as aliphatic or aromatic. In natural amino acids, the amine and carboxyl functional groups attached to the same carbon atom, i.e., an amino group is attached to the carbon in α-position relative to carboxyl group.

Any of the amino acids described herein may be in L configuration or in D configuration. In some embodiments, the amino acid is in L configuration. In some embodiments, the amino acid is in D configuration. The 20 natural amino acids are abbreviated herein as shown in Table A:

TABLE A

| Three-letter | One-letter | |
|---|---|---|
| abbreviation | abbreviation | Amino acid name |
| Ala | A | Alanine |
| Arg | R | Arginine |
| Asn | N | Asparagine |
| Asp | D | Aspartic acid |
| Cys | C | Cysteine |
| Gln | Q | Glutamine |
| Glu | E | Glutamic acid |
| Gly | G | Glycine |
| His | H | Histidine |
| Ile | I | Isoleucine |
| Leu | L | Leucine |
| Lys | K | Lysine |
| Met | M | Methionine |
| Phe | F | Phenylalanine |
| Pro | P | Proline |
| Ser | S | Serine |
| Thr | T | Threonine |
| Trp | W | Tryptophan |
| Tyr | Y | Tyrosine |
| Val | V | Valine |

### Self-Immolative Groups and Linker Moieties

Suitable examples of self-immolative groups include those described, for example, in Alouane, A. et al., “Self-immolative spacers: kinetic aspects, structure-property relationships, and applications”, Angew. Chem. Int. Ed., 2015, 54, 7492-7509 and Kolakowski, R. V. et al., “The methylene alkoxy carbamate self-immolative unit: Utilization of the targeted delivery of alcohol-containing payloads with antibody-drug conjugates”, Angew. Chem. Int. Ed., 2016, 55, 7948-7951.

Examples of self-immolative groups include the following groups of formulae (a)-(i):

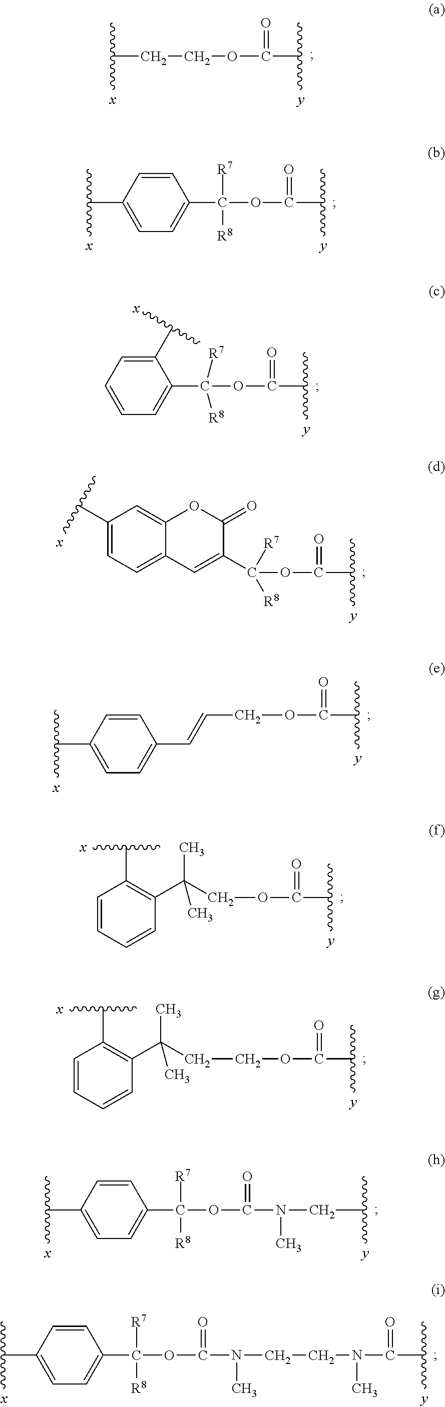

wherein x and y denote points of attachment to different L¹ or L², and R⁷ and R⁸ are each independently selected from H and C₁₋₆ alkyl.
   Groups and Moieties that are Formed by a “Click” Reaction

In some embodiments, the moiety formed by a click reaction is a reaction product of any one of the well-known click reactions, such as Huisgen cycloaddition (also known as [3+2] cycloaddition of alkynes and azides to form triazoles), Staudinger ligation (i.e., a reaction between an azide and a phosphine), a reaction of oxanorbornadienes and azides to from triazoles, an inverse-demand Diels-Alder reaction of tetrazines (e.g., dipyridyl tetrazines) and trans-cycloctynes, inverse-demand Diels-Alder reaction of tetrazines (e.g., monoaryl tetrazines) and norbornenes, a reaction of tetrazines and cyclopropenes, a reaction of cyclopropenes and nitrile imines, a photoinduced 1,3-dipolar cycloaddition of tetrazoles and alkenes, a 1,3-dipolar cycloaddition of nitrile oxides and norbornenes, a [4+1] cycloaddition isocyanides and tetrazines, or a 1,3-cycloaddition of nitrones and alkynes. In some embodiments, the moiety formed by a click reaction comprises a triazole.

In some embodiments, the moiety formed by a click reaction is selected from:

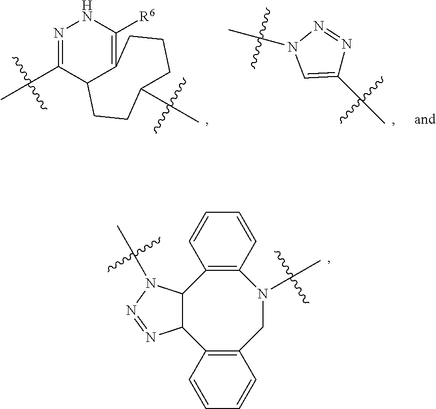

wherein R⁶ is selected from H and C₁₋₆ alkyl.

In some embodiments, the linker moieties (L¹)ₘ and (L²)ₙ moiety can be flexible or rigid, hydrophobic, hydrophilic, or amphiphilic. In some embodiments, length of the linker moiety is between about 10 Å to about 1,000 Å, from about 15 Å to about 800 Å, from about 20 Å to about 500 Å, from about 20 Å to about 400 Å, from about 20 Å to about 250 Å, from about 20 Å to about 20 Å to about 200 Å, or from about 20 Å to about 150 Å. For example, combined length of the linkers (L¹)ₘ and (L²)ₙ provides sufficient spacing between the Y¹ (e.g., affinity ligand or a group capable of reacting with the affinity ligand) and the radiotracer Y³. Either or both linker moieties can be flexible or rigid, hydrophobic, hydrophilic, or amphiphilic. In some embodiments, the combined length of the linker moieties is between about 10 Å to about 1,000 Å, from about 15 Å to about 800 Å, from about 20 Å to about 500 Å, from about 20 Å to about 400 Å, from about 20 Å to about 250 Å, from about 20 Å to about 20 Å to about 200 Å, or from about 20 Å to about 150 Å. Without being bound by any theory, it is believed that the length and physical properties of either linker moiety are selected for each affinity ligand and for each radiotracer such that upon binding of the affinity ligand to its biological target within the patient's body, the radiotracer can be effective as wither the imaging agent or the theranostic agent.

### Certain Embodiments of Formula (I):

In some embodiments, the compound of Formula (I) has formula:

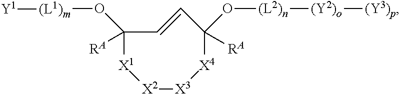

or a pharmaceutically acceptable salt thereof.

In some embodiments, the compound of Formula (I) has formula:

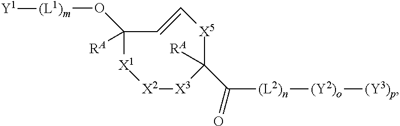

or a pharmaceutically acceptable salt thereof.

In some embodiments, the compound of Formula (I) has formula:

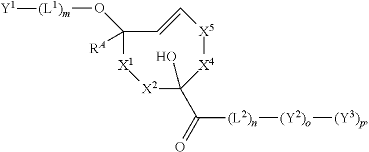

or a pharmaceutically acceptable salt thereof.

In some embodiments, the compound of Formula (I) has formula:

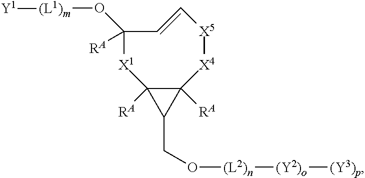

or a pharmaceutically acceptable salt thereof.

In some embodiments, the compound of Formula (I) has formula:

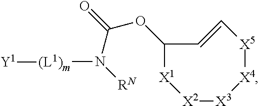

or a pharmaceutically acceptable salt thereof.

In some embodiments, the compound of Formula (I) has formula:

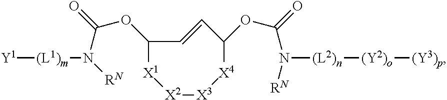

or a pharmaceutically acceptable salt thereof.

In some embodiments, the compound of Formula (I) has formula:

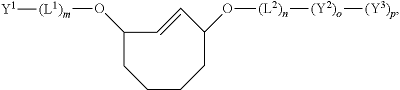

or a pharmaceutically acceptable salt thereof.

In some embodiments, the compound of Formula (I) has formula:

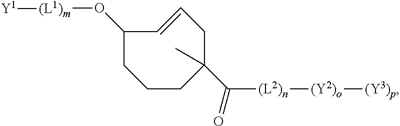

or a pharmaceutically acceptable salt thereof.

In some embodiments, the compound of Formula (I) has formula:

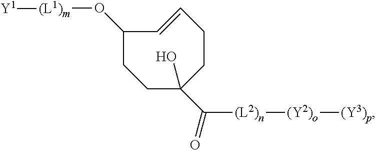

or a pharmaceutically acceptable salt thereof.
   Affinity Ligands and Groups Reactive with Affinity Ligands (Y¹)

In some embodiments, Y¹ is selected from NH₂, OH, and C(═O)OH. In some embodiments, Y¹ is NH₂. In some embodiments, Y¹ is OH. In some embodiments, Y¹ is C(═O)OH. In some embodiments, Y¹ is H.

In some embodiments, Y¹ is selected from a protected amino group, a protected hydroxyl group, and a protected carboxyl group. In some embodiments, Y¹ is a protected amino group. In some embodiments, Y¹ is a protected carboxyl group. In some embodiments, Y¹ is protected hydroxyl group.

### Protected Chemical Groups

In some embodiments, a skilled chemist would be able to select and implement any of the amine protecting groups, alcohol protecting groups, or carboxylic acid protecting groups of the present disclosure. The chemistry of protecting groups can be found, for example, in P. G. M. Wuts and T. W. Greene, Protective Groups in Organic Synthesis, 4^{th} Ed., Wiley & Sons, Inc., New York (2006) (which is incorporated herein by reference), including suitable examples of the protecting groups, and methods for protection and deprotection, and the selection of appropriate protecting groups.

Suitable examples of amine-protecting groups include carbobenzyloxy (Cbz) group, p-methoxybenzyl carbonyl (Moz or MeOZ), tert-butyloxycarbonyl (BOC) group, 9-fluorenylmethyloxycarbonyl (Fmoc), acetyl (Ac), benzoyl (Bz), benzyl (Bn) group, carbamate group, p-methoxybenzyl (PMB), 3,4-dimethoxybenzyl (DMPM), p-methoxyphenyl (PMP) group, tosyl (Ts) group, troc (trichloroethyl chloroformate), and nosyl group.

Suitable examples of alcohol-protecting groups include acetyl (Ac), benzoyl (Bz), benzyl (Bn), β-methoxyethoxymethyl ether (MEM), dimethoxytrityl, bis-(4-methoxyphenyl)phenylmethyl (DMT), methoxymethyl ether (MOM), methoxytrityl [(4-methoxyphenyl)diphenylmethyl](MMT), p-methoxybenzyl ether (PMB), methylthiomethyl ether, pivaloyl (Piv), tetrahydropyranyl (THP), tetrahydrofuran (THF), trityl (triphenylmethyl, Tr), silyl ether (most popular ones include trimethylsilyl (TMS), tert-butyldimethylsilyl (TBDMS), tri-iso-propylsilyloxymethyl (TOM), and triisopropylsilyl (TIPS) ethers, methyl ethers, and ethoxyethyl ethers (EE).

Suitable examples of carboxylic acid protecting groups include methyl esters, benzyl esters, tert-butyl esters, esters of 2,6-disubstituted phenols (e.g., 2,6-dimethylphenol, 2,6-diisopropylphenol, or 2,6-di-tert-butylphenol), silyl esters, orthoesters, and oxazoline.

### Reactive Chemical Groups

In some embodiments, Y¹ is a reactive chemical group. For example, the chemical group of Y¹ is capable of reacting with an affinity ligand as described herein. Suitable examples of reactive chemical groups include those reactive with OH, NH₂, C(O)OH, and SH functional groups, as well as bioorthogonal reactive groups.

Suitable examples of groups reactive with OH include the following groups:

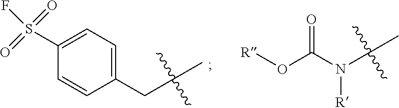

(R′ is H or C₁₋₃ alkyl, R″ is C₁₋₃ alkyl).

Suitable examples of groups reactive with SH include the following groups:

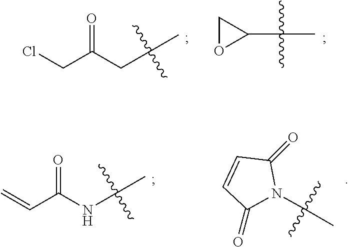

Suitable examples of groups reactive with NH₂ includes an activated ester of formula:

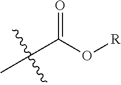

(R is, e.g., N-succinimidyl, N-benzotriazolyl, 4-nitrophenyl, or pentafluorophenyl).

Suitable examples of reactive chemical groups that are reactive in bioorthogonal chemical reactions include azide, alkene, alkyne, and tetrazine reactive groups, such as:

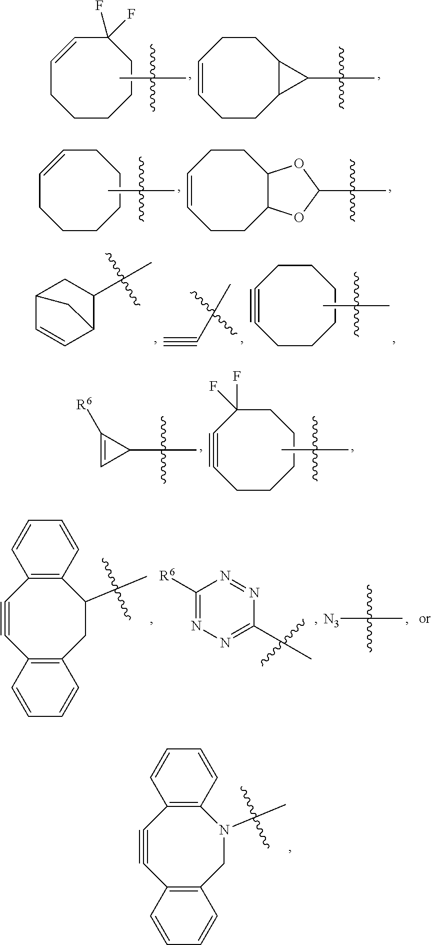

wherein R⁶ is selected from H and C₁₋₆ alkyl.

Other examples of reactive groups include a nitrone, an isocyanide, a diphenylphosphine, nitrile imine, a tetrazole, and a nitrile oxide.

### Affinity Ligands

Typically, affinity ligands are targeting groups and ligands that bind or react selectively with a receptor on the outside surface of a cell, or a biological target inside the cell. Suitable examples of extracellular targeting groups include RGD, cRGD, folic acid, hyaluronic acid, lectin, biotin, clathrin, caveolin, and transferrin. In some embodiments, the targeting ligand promotes transcytosis or endocytosis of the conjugate. In some embodiments, the targeting ligand is a peptide. In some embodiments, the peptide is EGF, CANF, or Angiopep-2.

In some embodiments, Y¹ is a biologically active protein. In some embodiments, Y¹ is a biologically active peptide. Suitable examples of bioactive peptides and proteins include a hormone, a transmembrane protein, a growth factor, an enzyme, and a structural protein. In some embodiments, Y¹ is a therapeutic peptide (e.g., containing 50 or fewer amino acids, 40 or fewer amino acids, 30 or fewer amino acids, 20 or fewer amino acids, or any number of amino acids that does not exceed 50). Suitable examples of peptides include Cpd86, ZPGG-72, ZP3022, MOD-6030, ZP2929, HM12525A, VSR859, NN9926, TTP273/TTP054, ZYOG1, MAR709, TT401, HM11260C, PB1023, ZP1848, ZP4207, ZP2929, dulaglutide, semaglutide, and ITCA. Other examples of therapeutic peptides are described in Fosgerau et al. Drug Discovery Today, 20 (1), 2015, 122-128; and Kaspar et al., Drug Discovery Today, 18 (17-18), 2013, 807-817.

Suitable examples of biologically active proteins include any one of the protein therapeutics described in Leader et al., Nature Reviews, 2008, 7, 21-39. In some embodiments, the therapeutic protein is a cytokine, such as transforming growth factor-beta (TGF-beta), an interferon (e.g., interferon-alpha, interferon-beta, interferon-gamma), a colony stimulating factor (e.g., granulocyte colony stimulating factor (GM-CSF)), and thymic stromal lymphopoietin (TSLP).

In some embodiments, the cytokine is an interleukin, such as interleukin-1, interleukin-2, interleukin-3, interleukin-4, interleukin-5, interleukin-6, interleukin-7, interleukin-8, interleukin-10, interleukin-12, interleukin-13, interleukin-15, interleukin-17, interleukin-18, interleukin-22, interleukin-23, and interleukin-35.

In some embodiments, the therapeutic protein is a polypeptide hormone, such as amylin, anti-Müllerian hormone, calcitonin, cholecystokinin, corticotropin, endothelin, enkephalin, erythropoietin (EPO), darbepoetin, follicle-stimulating hormone, gallanin, gastrin, ghrelin, glucagon, gonadotropin-releasing hormone, growth hormone-releasing hormone, hepcidin, human chorionic gonadotropin, growth hormone (GH), human growth hormone (hGH), inhibin, insulin, isophane insulin, insulin detemir, insulin glargine, pramlintide, pramlintide acetate, insulin-like growth factor, leptin, luteinizing hormone, luteinizing hormone releasing hormone, melanocyte stimulating hormone, motilin, orexin, oxytocin, pancreatic polypeptide, parathyroid hormone, prolactin, secretin, somatostatin, thrombopoietin, thyroid-stimulating hormone, vasoactive intestinal peptide, somatotropin, mecasermin, mecasermin rinfabate, human follicle-stimulating hormone, lutropin, teriparatide, exenatide, octreotide, dibotermin-α, bone morphogenetic protein 7, keratinocyte growth factor, platelet-derived growth factor, trypsin, nesiritide, and vasopressin.

In some embodiments, the therapeutic protein is tissue plasminogen activator (tPA), factor VIIa, factor VIII, factor IX, antithrombin III, protein C, drotrecogin-α, filgrastim, pegfilgrastim, sargramostim, lepirudin, bivalirudin, a madanin, a haemathrin, a variegin, an integrilin, or oprelvekin.

In some embodiments, the therapeutic protein is an enzyme. In some embodiments, the enzyme is agalsidase beta, imiglucerase, velaglucerase alfa, taliglucerase, alglucosidase alfa, laronidase, idursulfase, β-gluco-cerebrosidase, alglucosidase-α, laronidase, α-L-iduronidase, idursulphase, iduronate-2-sulphatase, galsulphase, agalsidase-β, human α-galactosidase A, α-1-proteinase, α-1-proteinase inhibitor, pancreatic enzyme, lactase, lipase, amylase, protease, adenosine deaminase, alteplase, reteplase, tenecteplase, urokinase, collagenase, human deoxyribonuclease I, dornase-α, hyaluronidase, papain, asparaginase (e.g. L-Asparaginase), rasburicase, streptokinase, anistreplase, or galsulfase.

In some embodiments, the therapeutic protein in useful in treating infectious disease. In some embodiments, the therapeutic protein useful in treating infectious disease is enfuvirtide.

In some embodiments, the therapeutic protein is useful in treating endocrine disorders (hormone deficiencies). In some aspects of these embodiments, the therapeutic protein is useful in treating diabetes, diabetes mellitus, diabetic ketoacidosis, hyperkalaemia, hyperglycemia, growth failure due to GH deficiency or chronic renal insufficiency, Prader-Willi syndrome, Turner syndrome, AIDS wasting or cachexia with antiviral therapy, growth failure in children with GH gene deletion or severe primary IGF1 deficiency, postmenopausal osteoporosis, severe osteoporosis, type 2 diabetes resistant to treatment with metformin and a sulphonylurea, or acromegaly.

In some embodiments, the therapeutic protein is useful in treating haemostasis and thrombosis. In some aspects of these embodiments, the therapeutic protein is useful in treating haemophilia A, haemophilia B, hereditary AT-III deficiency in connection with surgical or obstetrical procedures or for thromboembolism, venous thrombosis and purpura fulminans in patients with severe hereditary protein C deficiency, pulmonary embolism, myocardial infarction, acute ischaemic stroke, occlusion of central venous access devices, acute myocardial infarction, haemorrhage in patients with haemophilia A or B and inhibitors to factor VIII or factor IX, severe sepsis with a high risk of death, heparin-induced thrombocytopaenia, blood-clotting risk in coronary angioplasty, acute evolving transmural myocardial infarction, deep vein thrombosis, arterial thrombosis, occlusion of arteriovenous cannula, and thrombolysis in patients with unstable angina.

In some embodiments, Y¹ is a biologically active small-molecule drug. Small molecule drugs are low molecular weight organic compounds (typically about 2000 daltons or less). In some embodiments, the molecular weight of the drug molecule is in the range from about 200 to about 2000, from about 200 to about 1800, from about 200 to about 1600, from about 200 to about 1400, from about 200 to about 1200, from about 200 to about 1000, from about 200 to about 800, from about 200 to about 600 daltons, from about 300 to about 2000, from about 300 to about 1800, from about 300 to about 1600, from about 300 to about 1400, from about 300 to about 1200, from about 300 to about 1000, from about 300 to about 800, and/or from about 300 to about 600 daltons.

In some embodiments, the small-molecule drug is a cancer therapeutic. Suitable examples of small molecule drugs include antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), antifungal agents (e.g., butenafine, terbinafine, and naftifine), immunomodulating drugs (e.g., glatiramer acetate, fingolimod, teriflunomide, and dimethyl fumarate), and anti-mitotic agents (e.g., vincristine, vinblastine, paclitaxel, and maytansinoids).

Other suitable examples of small-molecule drugs include cytochalasin B, gramicidin D, doxorubicin, ethidium bromide, emetine, mitomycin, etoposide, tenoposide, colchicin, daunorubicin, dihydroxy anthracin dione, mithramycin, actinomycin D, 1-dehydrotestosterone, glucocorticoids, procaine, tetracaine, lidocaine, amphotericin B, propranolol, puromycin, and maytansinoids, e.g., maytansinol.

In some embodiments, Y¹ is selected from an antibody, an antibody fragment, a fusion antibody, a chimeric antibody, a nanobody, and an engineered antibody. Additional examples of affinity ligands also include nanoparticles, aptamers, and oligos.

### Antibodies and Antibody Fragments

In some embodiments, the antibody is useful in treating cancer. In some embodiments, the antibody useful in treating cancer is abagovomab, adecatumumab, afutuzumab, alacizumab pegol, altumomab pentetate, amatuximab, anatumomab mafenatox, apolizumab, arcitumomab, bavituximab, bectumomab, belimumab, bevacizumab, bivatuzumab mertansine, blinatumomab, brentuximab vedotin, cantuzumab mertansine, cantuzumab ravtansine, capromab pendetide, cetuximab, citatuzumab bogatox, cixutumumab, clivatuzumab tetraxetan, dacetuzumab, demcizumab, detumomab, drozitumab, ecromeximab, eculizumab, elotuzumab, ensituximab, epratuzumab, etaracizumab, farletuzumab, figitumumab, flanvotumab, galiximab, gemtuzumab ozogamicin, girentuximab, ibritumomab tiuxetan, imgatuzumab, ipilimumab, labetuzumab, lexatumumab, lorvotuzumab mertansine, nimotuzumab, ofatumumab, oregovomab, panitumumab, pemtumomab, pertuzumab, tacatuzumab tetraxetan, tositumomab, trastuzumab, totumumab, or zalutumumab.

In some embodiments, the antibody is useful in treating an inflammatory disease or condition. In some embodiments, the antibody useful in treating an inflammatory disease or condition is adalimumab, alemtuzumab, atlizumab, basiliximab, canakinumab, certolizumab, certolizumab pegol, daclizumab, muromonab, efalizumab, fontolizumab, golimumab, infliximab, mepolizumab, natalizumab, omalizumab, ruplizumab, ustekinumab, visilizumab, zanolimumab, vedolizumab, belimumab, otelixizumab, teplizumab, rituximab, ofatumumab, ocrelizumab, epratuzumab, eculizumab, or briakinumab.

In some embodiments, the antibody is specific to an antigen which is a biomarker of a disease or condition. In some embodiments, the disease or condition is cancer. Suitable examples of biomarkers include CD45, CD3, CD4, CD8, PD-1, PD-L¹, CD11b, F4/80, CD163, CD206, Ly6G, CD11c, and MHCII. Any other biomarker the presence of which in the cell (e.g., on the cell surface) is known in the art to be indicative of severity of the disease, or to be indicative of the presence of some disease state, can be used as an antigen for the antibody of any of the Formulae disclosed herein (e.g., Formula (I)). Some examples of cancer biomarkers include alpha fetoprotein (AFP), CA15-3, CA27-29, CA19-9, CA-125, calcitonin, calretinin, carcinoembryonic antigen, CD34, CD99MIC 2, CD117, chromogranin, chromosomes 3, 7, 17, and 9p21, cytokeratin (various types: TPA, TPS, Cyfra21-1), desmin, epithelial membrane antigen (EMA), factor VIII, CD31 FL1, glial fibrillary acidic protein (GFAP), gross cystic disease fluid protein (GCDFP-15), HMB-45, human chorionic gonadotropin (hCG), immunoglobulin, inhibin, keratin (various types), lymphocyte marker (various types, MART-1 (Melan-A), myo D1, muscle-specific actin (MSA), neurofilament, neuron-specific enolase (NSE), placental alkaline phosphatase (PLAP), prostate-specific antigen (PSA), PTPRC (CD45), S100 protein, smooth muscle actin (SMA), synaptophysin, thymidine kinase, thyroglobulin (Tg), thyroid transcription factor-1 (TTF-1), tumor M2-PK, and vimentin.

### Chelating Moieties (Y²)

In some embodiments, the chelating moiety is a small-molecule (e.g., having a molecular weight of about 2000 daltons or less). In some embodiments, the molecular weight of the chelating moiety in the range from about 200 to about 2000, from about 200 to about 1800, from about 200 to about 1600, from about 200 to about 1400, from about 200 to about 1200, from about 200 to about 1000, from about 200 to about 800, from about 200 to about 600 daltons, from about 300 to about 2000, from about 300 to about 1800, from about 300 to about 1600, from about 300 to about 1400, from about 300 to about 1200, from about 300 to about 1000, from about 300 to about 800, and/or from about 300 to about 600 daltons. In some embodiments, the chelating moiety comprises one or more amino acid residues (e.g., 1, 2, 3, 4, 5, 6, 7, 8, or 9 amino acid residues), e.g., as described herein. In some embodiments, the chelating moiety is acyclic. In some embodiments, the chelating moiety is cyclic (e.g., comprising 6, 7, 8, 9, 10, 11, 12, 13, 14, 15, 16, 17, 18, 19, 20, 21, 22, 23, 24, 25, or 26 atoms in the cyclic ring chain). In some embodiments, the chelating moiety comprises one or more heteroatoms in the acyclic or the cyclic ring chain. For example, the chelating moiety comprises 2, 3, or 4 nitrogen atoms in the acyclic or the cyclic ring chain. In some embodiments, the chelating moiety comprises one or more peripheral substituents selected from CH₂SO₃H, CH₂SO₂CH₃, CH₂NHSO(OH), CO(OH), CH₂CO(OH), CH₂CH₂CO(OH), CH₂NH₂, CH₂(C═O)NH₂, CH₂OPO(OH)(OH), CH₂CH₂OPO(OH)(OH), CH₂PO(OH)(OH), and CH₂CH₂PO(OH)(OH), and the like. Without being bond by any particular theory, it is believed that the one or more heteroatoms and the one or more peripheral group form a ligand sphere when chelated to a radiotracer (e.g., a metal-based radiotracer). In some embodiments, the chelating moiety is bifunctional, trifunctional, or tetrafunctional.

In some embodiments, the chelating moiety is selected from the group consisting of 1,4,7-triazacyclononanetriacetic acid (NOTA), 1,4,7,10-tetraazacyclododecane-1,4,7,10-tetraacetic acid (DOTA), 1,4,7-triazacyclononane-1-glutaric acid-4,7-diacetic acid (NODAGA), ethylene diamine tetra-acetic acid (EDTA), diethylene triaminepentaacetic acid (DTPA), cyclohexyl-1,2-diaminetetraacetic acid (CDTA), ethyleneglycol-0,0′-bis(2-aminoethyl)-N,N,N′,N′-tetraacetic acid (EGTA), N,N-bis(hydroxybenzyl)-ethylenediamine-N,N′-diacetic acid (HBED), triethylene tetramine hexaacetic acid (TTHA), hydroxyethyidiamine triacetic acid (HEDTA), and 1,4,8,11-tetraazacyclotetradecane-N,N′,N″,N′″-tetraacetic acid (TETA), 1,4,7,10-tetraaza-1,4,7,10-tetra-(2-carbamoyl methyl)-cyclododecane (TCMC), and desferrioxamine B (DFO).

In some embodiments, the chelating moiety is selected from HBED, TRAP, DATA, THP, TETA, TE2A, TE3A, diamSar, ATSM, DOTATOC, DO2A, TCMC, DEPA, DO3A, NODASA, NETA, NE3TA, TACN, NOPO, dedpa, octapa, azapa, decapa, phospa, BPCA, HBED, HBED, SHBED, BPCA, CP256, PCTA, PEPA, NEC, BAT, EC, SBAD, BAPEN, L², LICAM, L³, desferoxamine, oxine, and HMPAO. In some embodiments, the chelating moiety is any of the chelating moieties described in Brandt et al., 2018, J Nucl Med, 59, 1500-1506; and Price and Orvig, 2014, Chem Soc Rev, 43, 260-90, which are incorporated herein by reference in their entirety.

In some embodiments, Y² has formula:

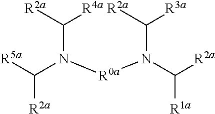

wherein:
R^{0a} is selected from —C₂₋₆ alkylene-, ring A, and C₁₋₃ alkylene-ring A-C₁₋₃ alkylene, wherein the alkylene and ring A are each optionally substituted with 1, 2, or 3 substituents independently selected from R^{1A}, and wherein R^{0a} is bound to the adjacent nitrogen atoms via the 1,2 or 1,3 positions on R^{0a};
each ring A is independently selected from C₃-C₁₀ cycloalkylene, 4-10 membered heterocycloalkylene, C₆-C₁₀ arylene, and 5-10 membered heteroarylene;
each R^{2a} is independently selected from H, —(C₁₋₃ alkyl)_{q}SO₃R^{A}, —(C₁₋₃ alkyl)_{q}SO₂R^{A}, —(C₁₋₃ alkyl)_{q}NHSO₂R^{A}, —(C₁₋₃ alkyl)_{q}CO₂R^{A}, —(C₁₋₃ alkyl)_{q}NR^{A}R^{B}, —(C₁₋₃ alkyl)_{q}(C═O)NR^{A}R^{B}, —(C₁₋₃ alkyl)_{q}OP(R^{A})O₂R^{B}, —(C₁₋₃ alkyl)_{q}OPO₃R^{A}R^{B}, —(C₁₋₃ alkyl)_{q}PO₃R^{A}R^{B}, and —(C₁₋₃ alkyl)_{q}(C═O)NHSO₂R^{A};
R^{1a} is selected from C(O)OR^{A}, P(O)(OR^{A})(OR^{B}), (C═O)NR^{A}R^{B}; and ring B, which is optionally substituted with 1, 2, or 3 substituents independently selected from R^{1B};
R^{3a} is selected from C(O)OR^{A}, P(O)(OR^{A})(OR^{B}), (C═O)NR^{A}R^{B}; and ring C, which is optionally substituted with 1, 2, or 3 substituents independently selected from R^{1C};
R^{4a} is selected from CO(OR^{A}), P(O)(OR^{A})(OR^{B}), (C═O)NR^{A}R^{B}; and ring D, which is optionally substituted with 1, 2, or 3 substituents independently selected from R^{1D};
R^{5a} is selected from CO(OR^{A}), P(O)(OR^{A})(OR^{B}), (C═O)NR^{A}R^{B}; and ring E, which is optionally substituted with 1, 2, or 3 substituents independently selected from R^{1E};
ring B is selected from 5-10 membered heteroaryl and a 5-10 membered heterocycloalkyl, each of which is optionally substituted with 1, 2, or 3 substituents independently selected from R^{1B};
ring C is selected from 5-10 membered heteroaryl and a 5-10 membered heterocycloalkyl, each of which is optionally substituted with 1, 2, or 3 substituents independently selected from R^{1C};
ring D is selected from 5-10 membered heteroaryl and a 5-10 membered heterocycloalkyl, each of which is optionally substituted with 1, 2, or 3 substituents independently selected from R^{1D};
ring E is selected from 5-10 membered heteroaryl and a 5-10 membered heterocycloalkyl, each of which is optionally substituted with 1, 2, or 3 substituents independently selected from R^{1E};
each R^{1A}, R^{1B}, R^{1C}, R^{1D}, and R^{1E} is independently selected from H, OH, NO₂, CN, halo, C₁₋₆ alkyl, C₂₋₆ alkenyl, C₂₋₆ alkynyl, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, amino, C₁₋₆ alkylamino, di(C₁₋₆ alkyl)amino, C₁₋₆ alkylsulfinyl, C₁₋₆ alkylsulfonyl, carbamyl, C₁₋₆ alkylcarbamyl, di(C₁₋₆ alkyl)carbamyl, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, di(C₁₋₆ alkyl)aminocarbonylamino, —(C₁₋₆ alkyl)_{q}SO₃R^{A}, —(C₁₋₆ alkyl)_{q}SO₂R^{A}, —(C₁₋₆ alkyl)_{q}NHSO₂R^{A}, —(C₁₋₆ alkyl)_{q}SO₂NR^{A}R^{B}, —(C₁₋₆ alkyl)_{q}CO₂R^{A}, —(C₁₋₆ alkyl)_{q}NR^{A}R^{B}, —(C₁₋₆ alkyl)_{q}(C═O)NR^{A}R^{B}, —(C₁₋₆ alkyl)_{q}OP(R^{A})O₂R^{B}, —(C₁₋₆ alkyl)_{q}OPO₃R^{A}R^{B}, —(C₁₋₆ alkyl)_{q}PO₃R^{A}R^{B}, and —(C₁₋₆ alkyl)_{q}(C═O)NHSO₂R^{A};
each q is independently 0 or 1; and
each R^{A} and R^{B} are independently hydrogen or C₁₋₆ alkyl.

In some embodiments, at least one of R^{1a}, R^{2a}, R^{3a}, R^{4a}, and R^{5a} is connected to L² of the (L²)ₙ linker, e.g., covalently or non-covalently.

In some embodiments, ring A is phenylene, optionally substituted with R^{1A}.

In some embodiments, ring A is 5-6 membered heteroarylene, optionally substituted with R^{1A}.

In some embodiments, ring A is C₃-C₁₀ cycloalkylene, optionally substituted with R^{1A}.

In some embodiments, ring A is cyclopentylene or cyclohexylene, optionally substituted with R^{1A}.

In some embodiments, ring A is a moiety of formula:

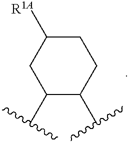

In some embodiments, ring A is 4-10 membered heterocycloalkylene, optionally substituted with R^{1A}.

In some embodiments, R^{0a} is C₂₋₆ alkylene, optionally substituted with R^{1A}.

In some embodiments, R^{0a} is 1,2-ethylene, optionally substituted with R^{1A}.

In some embodiments, R^{0a} is a moiety of formula:

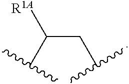

In some embodiments, each R^{1A} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).

In some embodiments, ring A is selected from any one of the following moieties:

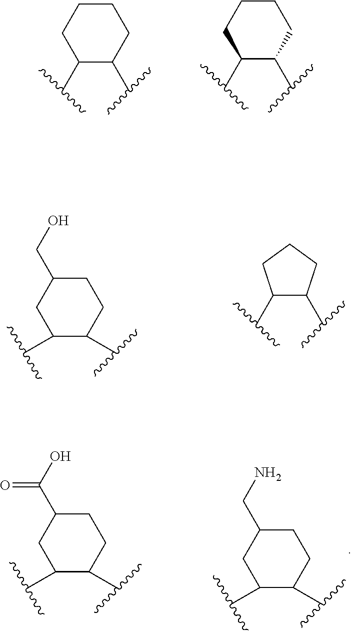 [SMILES: *C1CCCCC1* ; *[C@H]1CCCC[C@@H]1* ; *C1CCC(CO)CC1* ; *C1CCCC1* ; *C1CCC(C(=O)O)CC1* ; *C1CCC(CN)CC1*]

In some embodiments, ring A is selected from any one of the following moieties:

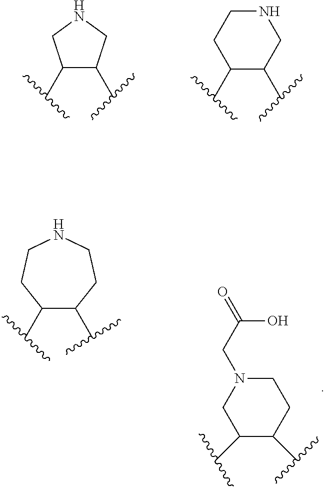 [SMILES: *C1CNCC1* ; *C1CCNCC1* ; *C1CCNCCC1* ; *C1CCN(CC(=O)O)CC1*]

In some embodiments, R^{0a} is selected from any one of the following moieties:

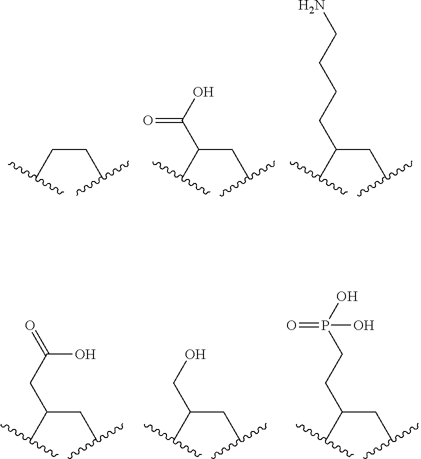

In some embodiments, R^{0a} is selected from the group consisting of a substituted or unsubstituted alkylene, such as 1,3-propylene or 1,2-ethylene, as shown below:

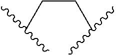 [SMILES: *CC*]

or 2,3-propylene-1-carboxylate, as shown below,

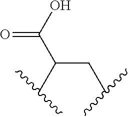 [SMILES: *CC(*)C(=O)O]

or 3,4-butylene-1-carboxylic acid, as shown below,

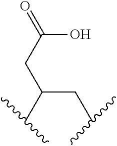 [SMILES: *CC(*)CC(=O)O]

or 1-hydroxy-3,4-butylene, as shown below,

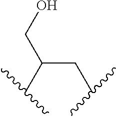 [SMILES: *CC(*)CO]

or 1-amino-5,6-hexylene, as shown below,

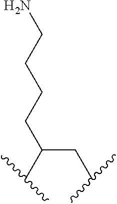 [SMILES: *CC(*)CCCCN]

substituted or unsubstituted cycloalkylene, such as those shown below, for example, cis- or trans-1,2-cyclohexylene, as shown below,

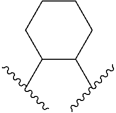 [SMILES: *C1CCCCC1*]

or trans-1,2-cyclohexylene, as shown below,

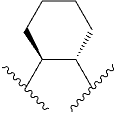 [SMILES: *[C@H]1CCCC[C@@H]1*]

or cis- or trans-1,2-cyclopentylene, as shown below,

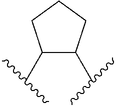 [SMILES: *C1CCCC1*]

substituted or unsubstituted monocyclic heterocyclyl, such as 2,5-dihydro-1H-pyrrolene, as shown below

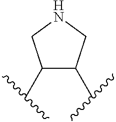 [SMILES: *C1CNCC1*]

1,2,3,6-tetrahydropyridinene, as shown below,

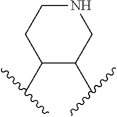 [SMILES: *C1CCNCC1*]

2,3,6,7-tetrahydro-1H-azepinene, as shown below,

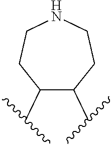 [SMILES: *C1CCNCCC1*]

In some embodiments, each R^{2a} is independently selected from H, CH₂SO₃H, CH₂SO₂CH₃, CH₂NHSO(OH), CO(OH), CH₂CO(OH), CH₂CH₂CO(OH), CH₂NH₂, CH₂(C═O)NH₂, CH₂OPO(OH)(OH), CH₂CH₂OPO(OH)(OH), CH₂PO(OH)(OH), and CH₂CH₂PO(OH)(OH).

In some embodiments, each R^{2a} is H.

In some embodiments, at least one R^{2a} is selected from CH₂SO₃H, CH₂SO₂CH₃, CH₂NHSO(OH), CO(OH), CH₂CO(OH), CH₂CH₂CO(OH), CH₂NH₂, CH₂(C═O)NH₂, CH₂OPO(OH)(OH), CH₂CH₂OPO(OH)(OH), CH₂PO(OH)(OH), and CH₂CH₂PO(OH)(OH).

In some embodiments, R^{1a} is ring B, optionally substituted with 1, 2, or 3 substituents independently selected from R^{1B}.

In some embodiments, R^{3a} is ring C, optionally substituted with 1, 2, or 3 substituents independently selected from R^{1C}.

In some embodiments, R^{4a} is ring D, optionally substituted with 1, 2, or 3 substituents independently selected from R^{1D}.

In some embodiments, R^{5a} is ring D, optionally substituted with 1, 2, or 3 substituents independently selected from R^{1D}.

In some embodiments, ring B is 5-6 membered heteroaryl optionally substituted with 1, 2, or 3 substituents independently selected from R^{1B}.

In some embodiments, C is 5-6 membered heteroaryl optionally substituted with 1, 2, or 3 substituents independently selected from R^{1C}.

In some embodiments, ring D is 5-6 membered heteroaryl optionally substituted with 1, 2, or 3 substituents independently selected from R^{1D}.

In some embodiments, ring E is 5-6 membered heteroaryl optionally substituted with 1, 2, or 3 substituents independently selected from R^{1E}.

In some embodiments, the 5-6 membered heteroaryl of ring B, ring C, ring D, or ring E is selected from any one of the following moieties:

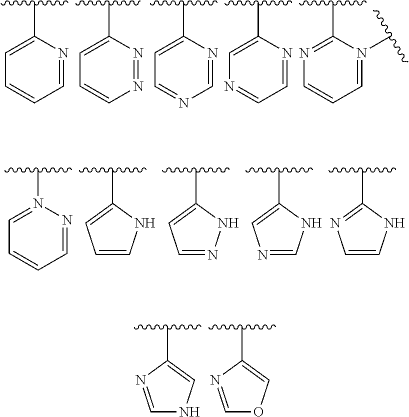

In some embodiments, ring B is 5-6 membered heterocycloalkyl, optionally substituted with 1, 2, or 3 substituents independently selected from R^{1B}.

In some embodiments, ring C is 5-6 membered heterocycloalkyl optionally substituted with 1, 2, or 3 substituents independently selected from R^{1C}.

In some embodiments, ring D is 5-6 membered heterocycloalkyl optionally substituted with 1, 2, or 3 substituents independently selected from R^{1D}.

In some embodiments, ring E is 5-6 membered heterocycloalkyl optionally substituted with 1, 2, or 3 substituents independently selected from R^{1E}.

In some embodiments, the 5-6 membered heterocycloalkyl of ring B, ring C, ring D, or ring E is selected from any one of the following moieties:

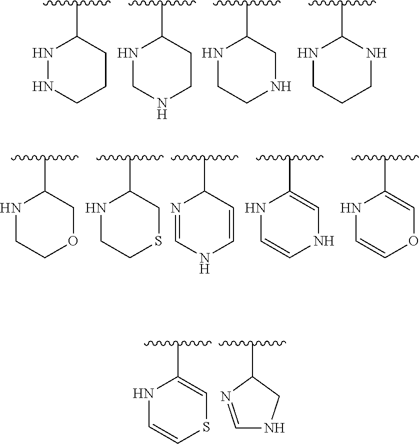

In some embodiments, each R^{B} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).

In some embodiments, each R^{1C} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).

In some embodiments, each RD is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).

In some embodiments, each R^{1E} is independently selected from H, OH, CN, halo, C₁₋₆ haloalkyl, C₁₋₆ alkoxy, C₁₋₆ haloalkoxy, —(C₁₋₃ alkyl)_{q}OH, —(C₁₋₃ alkyl)_{q}CN, —(C₁₋₆ alkyl)_{q}SO₂(OH), —(C₁₋₆ alkyl)_{q}C(O)(OH), —(C₁₋₆ alkyl)_{q}OPO(OH)(OH), and —(C₁₋₆ alkyl)_{q}PO(OH)(OH).

In some embodiments, the 5-6 membered heteroaryl of ring B, ring C, ring D, or ring E is selected from any one of the following moieties:

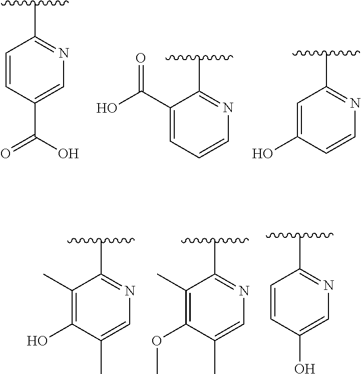

In some embodiments, Y² has any one of the following formulae:

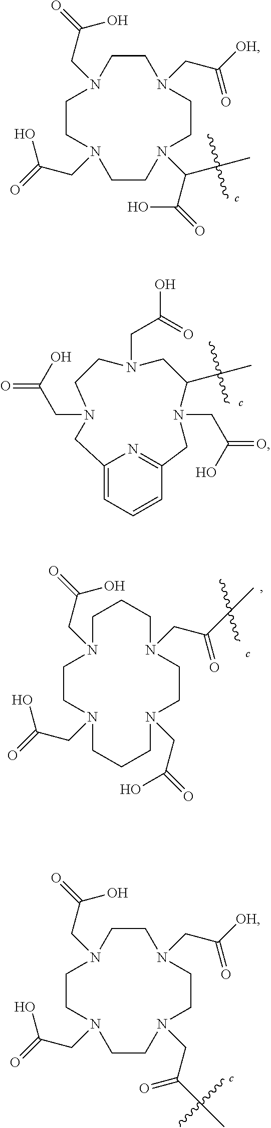 [SMILES: *C(C(=O)O)N1CCN(CC(=O)O)CCN(CC(=O)O)CCN(CC(=O)O)CC1 ; *C1CN(CC(=O)O)CCN(CC(=O)O)Cc2cccc(n2)CN1CC(=O)O ; *C(=O)CN1CCCN(CC(=O)O)CCN(CC(=O)O)CCCN(CC(=O)O)CC1 ; *C(=O)CN1CCN(CC(=O)O)CCN(CC(=O)O)CCN(CC(=O)O)CC1]

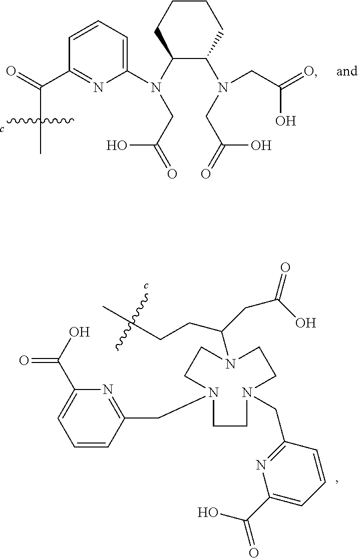 [SMILES: *C(=O)c1cccc(N(CC(=O)O)[C@H]2CCCC[C@@H]2N(CC(=O)O)CC(=O)O)n1 ; *CCC(CC(=O)O)N1CCN(Cc2cccc(C(=O)O)n2)CCN(Cc2cccc(C(=O)O)n2)CC1]

wherein c indicates a point of attachment to L² of (L²)ₙ linker.

In some embodiments, the compound of Formula (I) has formula:

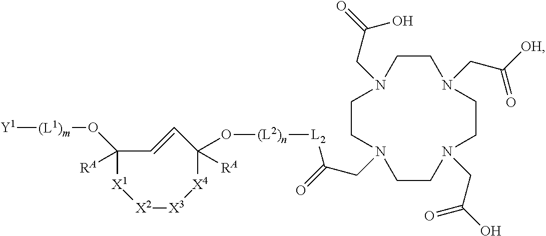

or a pharmaceutically acceptable salt thereof.

In some embodiments, the compound of Formula (I) has formula:

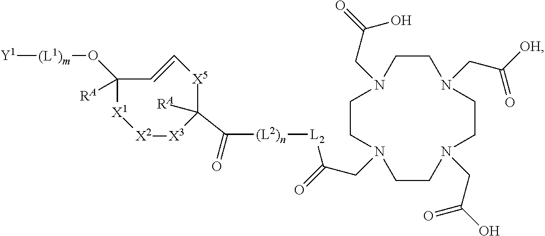

or a pharmaceutically acceptable salt thereof.

In some embodiments, the compound of Formula (I) has formula:

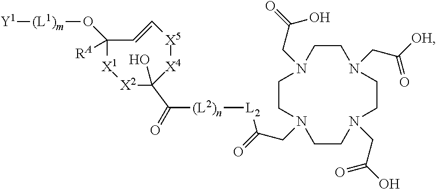

or a pharmaceutically acceptable salt thereof.

### Radiotracers (Y³)

In some embodiments, Y³ comprises a metal radioisotope. In some embodiments, the radioisotope is selected from the group consisting of ³H, ¹¹C, ¹⁴C, ¹⁸F, ³²P, ³⁵S, ³⁶Cl, ⁴⁴Sc, ⁴⁷Sc, ⁵¹Cr, ⁵²Fe, ^{52g}Mn, ⁵⁷Co, ⁵⁸Co, ⁵⁹Fe, ⁶⁴Cu, ⁶⁷Cu, ⁶⁷Ga, ⁶⁸Ga, ⁷⁵Se, ⁷⁶Br, ⁷⁷Br, ⁸²Rb, ⁸⁶Y, ⁸⁹Zr, ⁹⁰Y, ^{99m}Tc, ¹¹¹In, ^{14m}In, ¹²³I, ¹²⁴I, ¹²⁵I, ¹³¹I, ¹³³Xe, ¹⁵²Eu, ¹⁵³Sm, ¹⁶⁶Ho ¹⁷⁷Lu, ¹⁸⁶Re, ¹⁸⁸Re, ²⁰¹Tl, ²⁰³Pb, ²¹⁰At, ²¹¹At, ²¹²Bi, ²¹²Pb, ²¹²Bi, ²¹³Bi, ²²³Ra, ²²⁵Ac, and ²²⁷Th. In some embodiments, Y³ comprises a PET imagable radioisotope. Suitable examples of PET imagable radioisotopes include ¹¹C, ¹⁸F, ⁴⁴Sc, ^{52g}Mn, ⁶⁴Cu, ⁶⁸Ga, ⁸²Rb, ⁸⁶Y, ⁸⁹Zr, ¹²³I, ¹²⁴I, ¹²⁵I and ¹³¹I. In some embodiments, o is 0, and Y³ comprises a radiotracer moiety selected from ¹⁸F, ¹²³I, ¹²⁵I, ¹³¹I, ¹¹CN, ¹¹C(═O)NH₂, ¹¹CH₃—, ¹¹CH₃O—, ¹⁸FCH₂CH₂—, ¹⁸FCH₂CH₂O—, ¹⁸FCH₂CH₂CH₂O—, and phenyl substituted in 2, 3, and/or 4 position with independently selected ¹⁸F, ¹²³I, ¹²⁵I, ¹³¹I, ¹¹CN, ¹¹C(═O)NH₂, ¹¹CH₃—, ¹¹CH₃O—, ¹⁸FCH₂CH₂—, ¹⁸FCH₂CH₂O—, or ¹⁸FCH₂CH₂CH₂O—.

In some embodiments, Y³ comprises a SPECT imagable radioisotope.

Suitable examples of SPECT imagable radioisotopes include ⁶⁷Ga, ^{99m}Tc, ¹¹¹In, ^{114m}In, ¹²³I, ¹³³Xe, ¹⁷⁷Lu, and ²⁰¹Tl. In some embodiments, Y³ comprises a toxic radioisotope (useful, e.g., in theranostic applications such as a cancer therapy). Suitable examples of toxic radioisotopes include ⁴⁷Sc, ⁹⁰Y, ^{114m}In, ¹³¹I, ¹⁷⁷Lu, ¹⁸⁶Re, ¹⁸⁸Re, ²¹¹At, ²¹²Bi, ²¹²Pb, ²¹²Bi, ²¹³Bi, ²²³Ra, ²²⁵Ac, and ²²⁷Th. In some embodiments, the radioisotopes provided herein are useful as imaging agents in one or more of the methods provided herein. In addition, one or more of the radioisotopes provided herein may also be useful in one or more therapeutic applications, for example, when administered to a subject in a therapeutically effective amount. For example, ¹³¹I and ⁶⁴Cu may be useful as imaging agents (e.g., as non-toxic and/or non-therapeutic radioisotopes) when administered to the subject at low concentrations (e.g., 5 mCi) and may also be useful as therapeutic agents (i.e., as toxic radioisotopes and/or therapeutic radioisotopes) when administered to the subject at a higher concentration. In some embodiments, the one or more radioisotopes are independently directly or indirectly (e.g., through a chelator) bound to the compounds of Formula (A) or Formula (I) provided herein.

In some embodiments, Y³ is a paramagnetic ion. In some embodiments, the paramagnetic ion is selected from the group consisting of chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III).

In some embodiments, Y³ is an x-ray imaging agent. In some embodiments, the x-ray imaging agent is selected from the group consisting of lanthanum (III), gold (III), lead (II), bismuth (III), and an iodinated x-ray imaging agent (e.g, diatrizoate, ioxaglate, metrizoate, iopamidol, iohexol, ioxilan, iopromide, iodixanol, and ioversol).

In some embodiments, the compound of Formula (A) and/or Formula (I) comprises R¹⁰ in place of —(Y²)ₒ—(Y³)ₚ. In some embodiments, R¹⁰ is a fluorophore or a fluorescent group. Suitable examples of fluorophores include any fluorescent chemical compound that can re-emit light upon light excitation. The fluorophores can by excited by a light of a wavelength form about 300 nm to about 800 nm, and then emit light of a wavelength from about 350 nm to about 770 nm (e.g., violet, blue, cyan, green, yellow, orange or red light), which can be detected by fluorescent imaging devices, including the ability to measure the intensity of the fluorescence. Suitable examples of fluorophores include AF488, hydroxycoumarin blue, methoxycoumarin blue, Alexa fluor blue, aminocoumarin blue, Cy2 green (dark), FAM green (dark), Alexa fluor 488 green (light), Fluorescein FITC green (light), Alexa fluor 430 green (light), Alexa fluor 532 green (light), HEX green (light), Cy3 yellow, TRITC yellow, Alexa fluor 546 yellow, Alexa fluor 555 3 yellow, R-phycoerythrin (PE) 480; yellow, Rhodamine Red-X orange, Tamara red, Cy3.5 581 red, Rox red, Alexa fluor 568 red, Red 613 red, Texas Red red, Alexa fluor 594 red, Alexa fluor 633 red, Allophycocyanin red, Alexa fluor 633 red, Cy5 red, Alexa fluor 660 red, Cy5.5 red, TruRed red, Alexa fluor 680 red, and Cy7 red. Absorbance and emission wavelengths of these fluorophores are well known in the art.

In some embodiments, Y²-Y³ has any one of the following formulae:

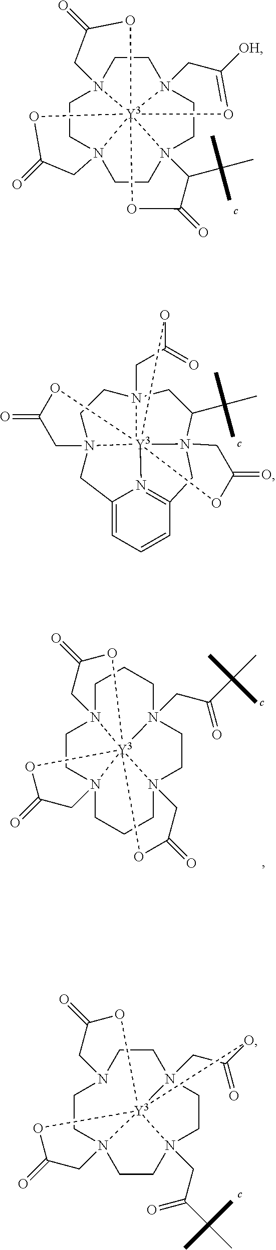

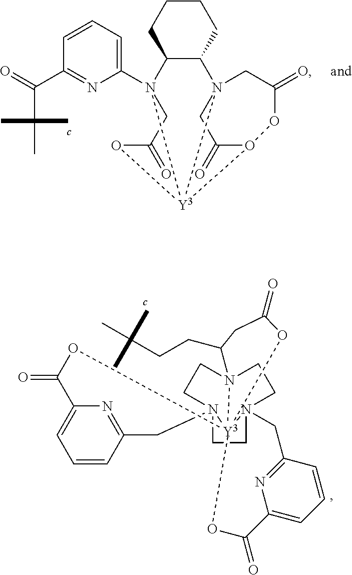

wherein c indicates a point of attachment to L².

In some embodiments, the compound of Formula (I) has formula.

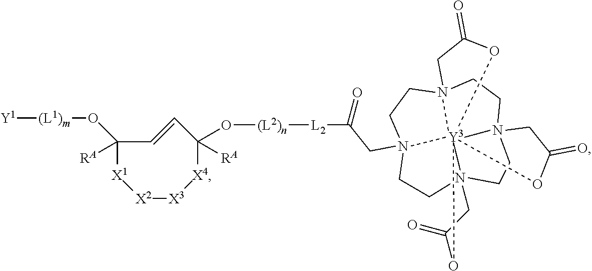

or a pharmaceutically acceptable salt thereof.

In some embodiments, the compound of Formula (I) has formula:

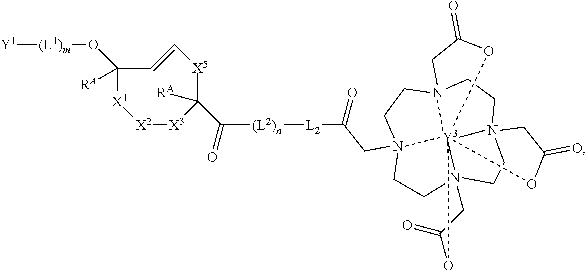

or a pharmaceutically acceptable salt thereof.

In some embodiments, the compound of Formula (I) has formula:

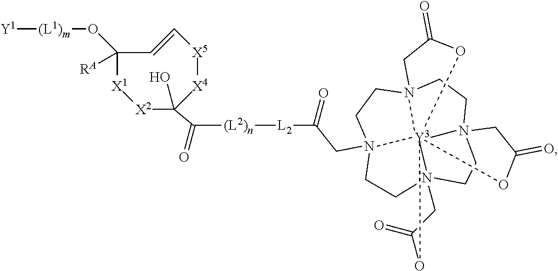

or a pharmaceutically acceptable salt thereof.

In some embodiments, the compound of Formula (I) has formula:

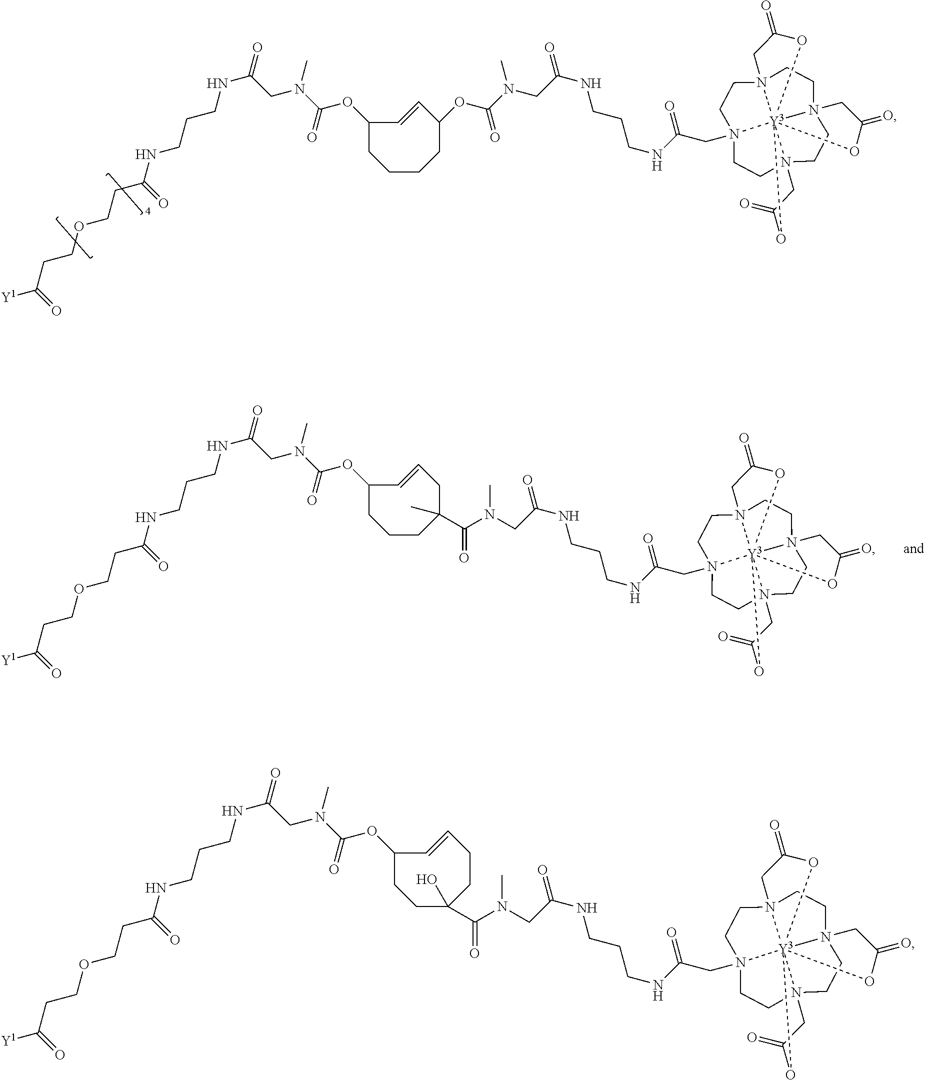

or a pharmaceutically acceptable salt thereof,
Y¹ is an affinity ligand.

In some embodiments, the compound of Formula (I) has formula:

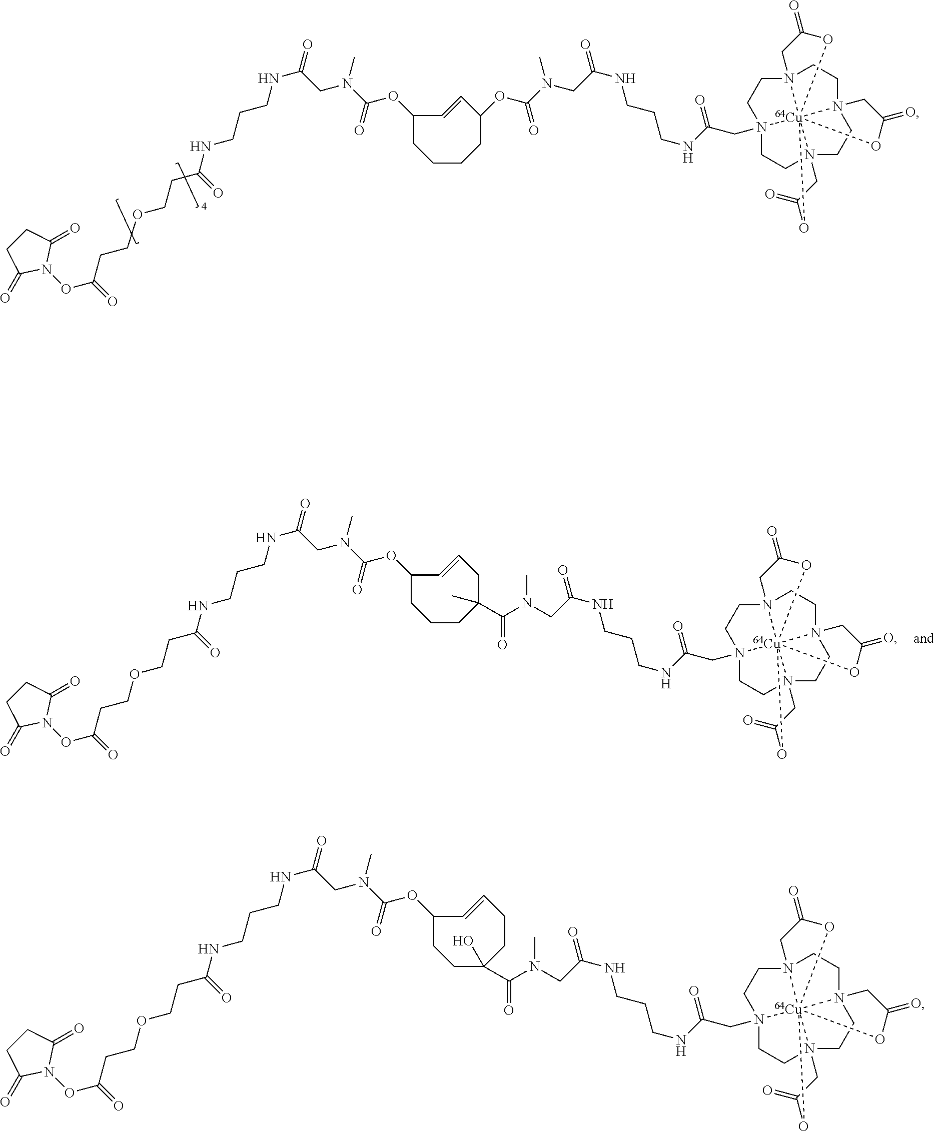

or a pharmaceutically acceptable salt thereof.

In some embodiments, the compound of Formula (I) has formula:

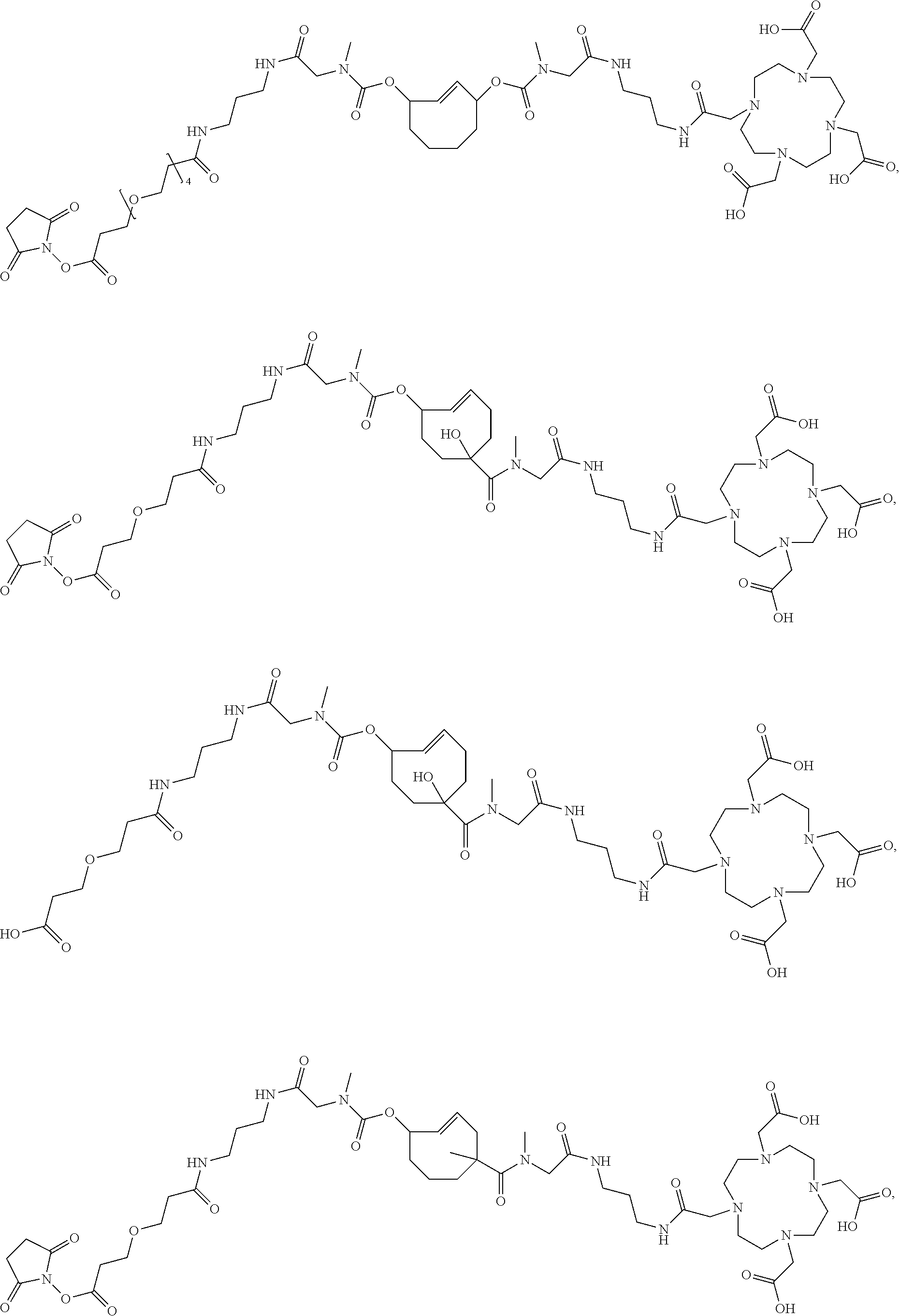

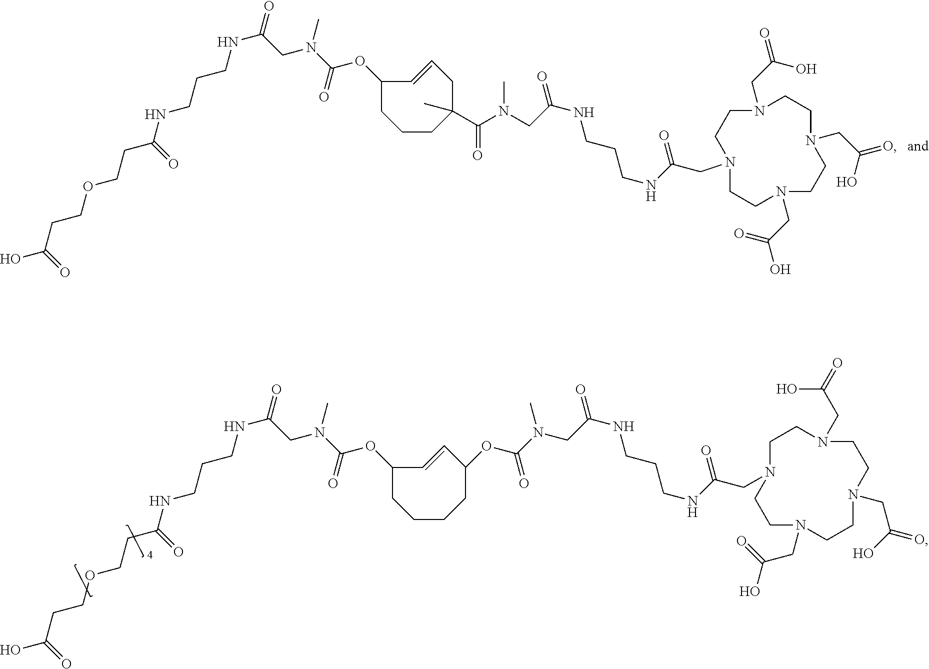

or a pharmaceutically acceptable salt thereof.

### Tetrazine (“Tz”)-Containing Reagents

In some embodiments, the present disclosure provides a compound of Formula (II) (e.g., useful as an antidote to compound of Formula (A) or (I) as described herein):

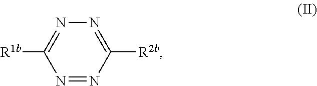

or a pharmaceutically acceptable salt thereof, wherein:
R^{1b} and R^{2b} are each independently selected from H, C₁₋₆ alkyl, C₆₋₁₀ aryl, 5-6-membered heteroaryl, C₃₋₁₀ cycloalkyl, 4-7-membered heterocycloalkyl, wherein said C₁₋₆ alkyl, C₆₋₁₀ aryl, 5-6-membered heteroaryl, C₃₋₁₀ cycloalkyl, and 4-7-membered heterocycloalkyl are each optionally substituted with 1, 2, 3, or 4 substituents independently selected from R^{2c};
each R^{2c} is independently selected from halo, CN, NO₂, C₁₋₆ alkyl, C₁₋₆ haloalkyl, C₂₋₆ alkenyl, C₂₋₆ alkynyl, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}; wherein said C₁₋₆ alkyl, C₂₋₆ alkenyl, and C₂₋₆ alkynyl are each optionally substituted with 1, 2 or 3 substituents independently selected from CN, NO₂, C₁₋₆ alkyl, C₁₋₆ haloalkyl, C₂₋₆ alkenyl, C₂₋₆ alkynyl, OR^{a1}, C(O)R^{b1}, C(O)NR^{c1}R^{d1}, C(O)OR^{a1}, OC(O)R^{b1}, OC(O)NR^{c1}R^{d1} NR^{c1}R^{d1}, NR^{c1}C(O)R^{b1}, NR^{c1}C(O)OR^{a1};
each R^{a1}, R^{b1}, R^{c1}, and R^{d1} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene are optionally substituted with 1, 2, 3, 4, or 5 substituents independently selected from R^{g}; and
each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, R¹ is H. In some embodiments, R² is H.

In some embodiments, R¹ and R² are each independently selected from H, C₁₋₆ alkyl, C₆₋₁₀ aryl, and 5-6-membered heteroaryl, wherein said C₁₋₆ alkyl, C₆₋₁₀ aryl, and 5-6-membered heteroaryl are each optionally substituted with 1 or 2 independently selected R^{2c}.

In some embodiments, R¹ is H, and R² is selected from C₁₋₆ alkyl, C₆₋₁₀ aryl, 5-6-membered heteroaryl, C₃₋₁₀ cycloalkyl, 4-7-membered heterocycloalkyl, wherein said C₁₋₆ alkyl, C₆₋₁₀ aryl, 5-6-membered heteroaryl, C₃₋₁₀ cycloalkyl, and 4-7-membered heterocycloalkyl are each optionally substituted with 1, 2, 3, or 4 substituents independently selected from R^{2c}.

In some embodiments, R¹ is C₆₋₁₀ aryl substituted with 1 or 2 independently selected R^{2c}. In some embodiments, R² is C₆₋₁₀ aryl substituted with 1 or 2 independently selected R^{2c}.

In some embodiments, R¹ is 5-6-membered heteroaryl 1 or 2 independently selected R^{2c}. In some embodiments, R² is 5-6-membered heteroaryl 1 or 2 independently selected R^{2c}.

In some embodiments, R¹ is C₁₋₆ alkyl substituted with OR^{a1}, C(O)OR^{a1}, or NR^{c1}R^{d1}. In some embodiments, R² is C₁₋₆ alkyl substituted with OR^{a1}, C(O)OR^{a1}, or NR^{c1}R^{d1}.

In some embodiments, R¹ is C₆₋₁₀ aryl substituted with OR^{a1}, C(O)OR^{a1}, or NR^{c1}R^{d1}. In some embodiments, R² is C₆₋₁₀ aryl substituted with OR^{a1}, C(O)OR^{a1}, or NR^{c1}R^{d1}.

In some embodiments, R¹ is 5-6-membered heteroaryl substituted with OR^{a1}, C(O)OR^{a1}, or NR^{c1}R^{d1}. In some embodiments, R² is 5-6-membered heteroaryl substituted with OR^{a1}, C(O)OR^{a1}, or NR^{c1}R^{d1}.

In some embodiments, R¹ and R² are each C₁₋₆ alkyl.

In some embodiments, R¹ and R² are each 5-6-membered heteroaryl.

In some embodiments, R¹ is C₁₋₆ alkyl and R² is 5-6-membered heteroaryl.

In some embodiments, the compound of Formula (II) has formula:

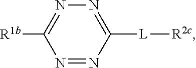

or a pharmaceutically acceptable salt thereof, wherein:
L is C₁₋₃ alkylene, C₆₋₁₀ arylene, C₃₋₁₀ cycloalkylene, 4-7-membered heterocycloalkylene, or 5-6-membered heteroarylene, each or which is optionally substituted with 1 or 2 substituents independently selected from halo, C₁₋₆ alkoxy, and C₁₋₆ haloalkoxy.

In some embodiments, R^{2c} is selected from OH, NH₂, and C(O)OH.

In some embodiments, L is C₁₋₃ alkylene. In some embodiments, L is C₆₋₁₀ arylene. In some embodiments, L is C₃₋₁₀ cycloalkylene. In some embodiments, L is 4-7-membered heterocycloalkylene. In some embodiments, L is -6-membered heteroarylene. In some embodiments, R^{2c} is OH. In some embodiments, R^{2c} is NH₂. In some embodiments, R^{2c} is C(O)OH.

In some embodiments, the compound of Formula (II) has formula:

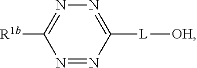

or a pharmaceutically acceptable salt thereof.

In some embodiments, the compound of Formula (II) has formula:

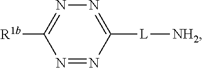

or a pharmaceutically acceptable salt thereof.

In some embodiments, the compound of Formula (II) has formula:

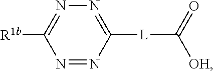

or a pharmaceutically acceptable salt thereof.

In some embodiments, the compound of Formula (II) has formula:

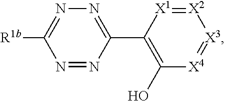

or a pharmaceutically acceptable salt thereof. In some embodiments, X¹, X², X³, and X⁴ are each independently selected from CH, N, and CR^{2c}. In some embodiments, X¹, X², X³, and X⁴ are each independently selected from CH and N. In some embodiments, only one of X¹, X², X³, and X⁴ is N. In some embodiments, no more than two of X¹, X², X³, and X⁴ are N.

In some embodiments, the compound of Formula (II) has formula:

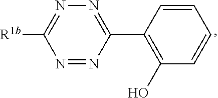

or a pharmaceutically acceptable salt thereof.

In some embodiments, the compound of Formula (II) has formula:

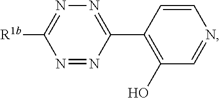

or a pharmaceutically acceptable salt thereof.

In some embodiments, the compound of Formula (II) has formula:

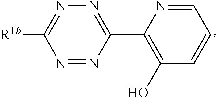

or a pharmaceutically acceptable salt thereof.

In some embodiments, R^{1b} is selected from C₁₋₆ alkyl, C₆₋₁₀ aryl, and 5-6-membered heteroaryl (e.g., optionally substituted with R^{2c}). In some embodiments, R^{1b} is H.

In some embodiments, the compound of Formula (II) has formula:

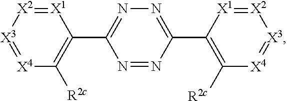

or a pharmaceutically acceptable salt thereof.

In some embodiments, the compound of Formula (II) is selected from any one of the following compounds:

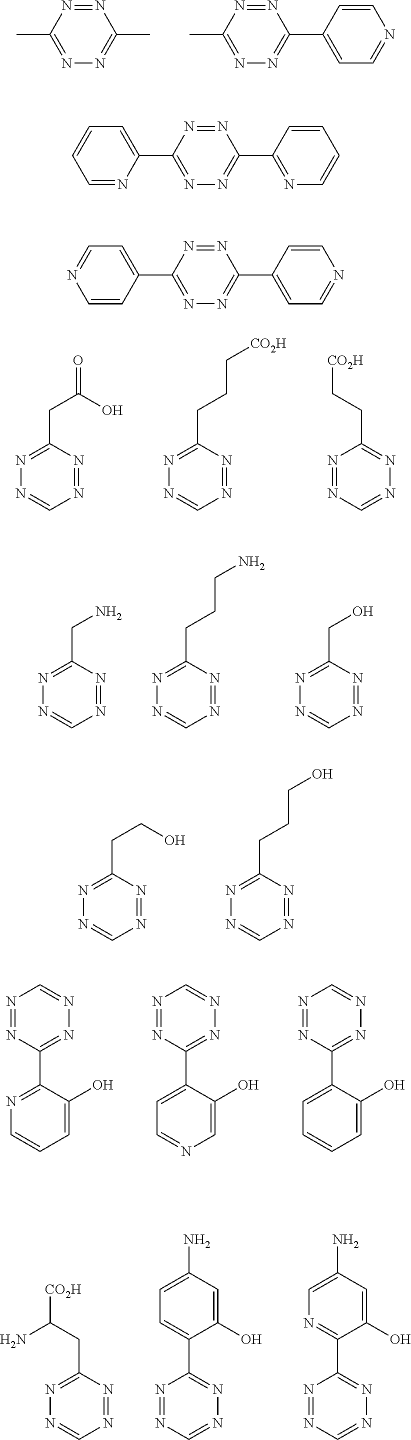

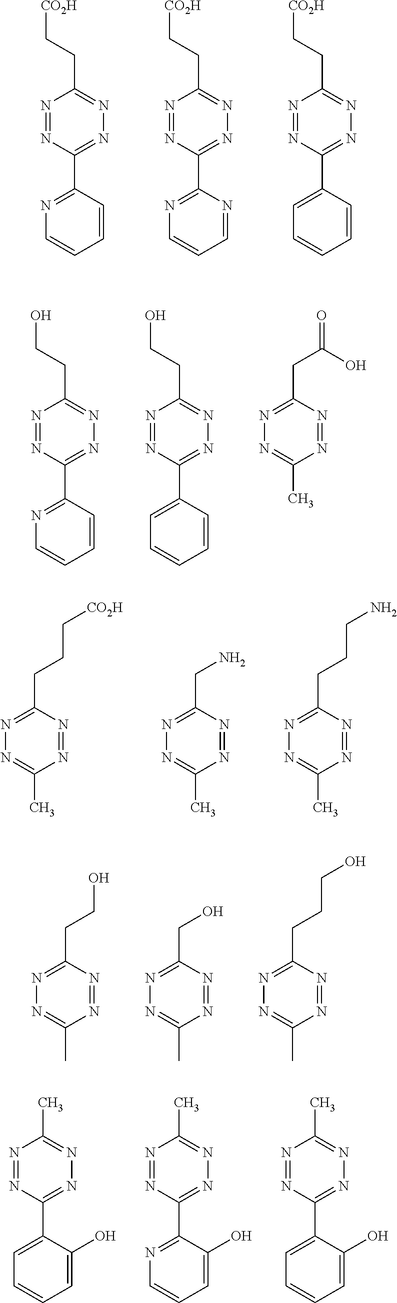

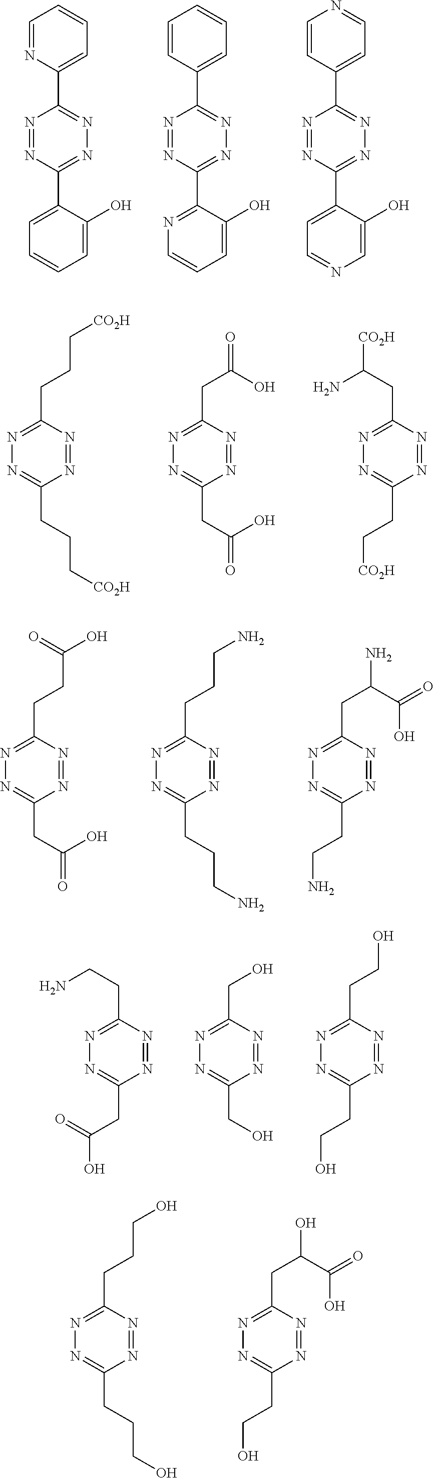

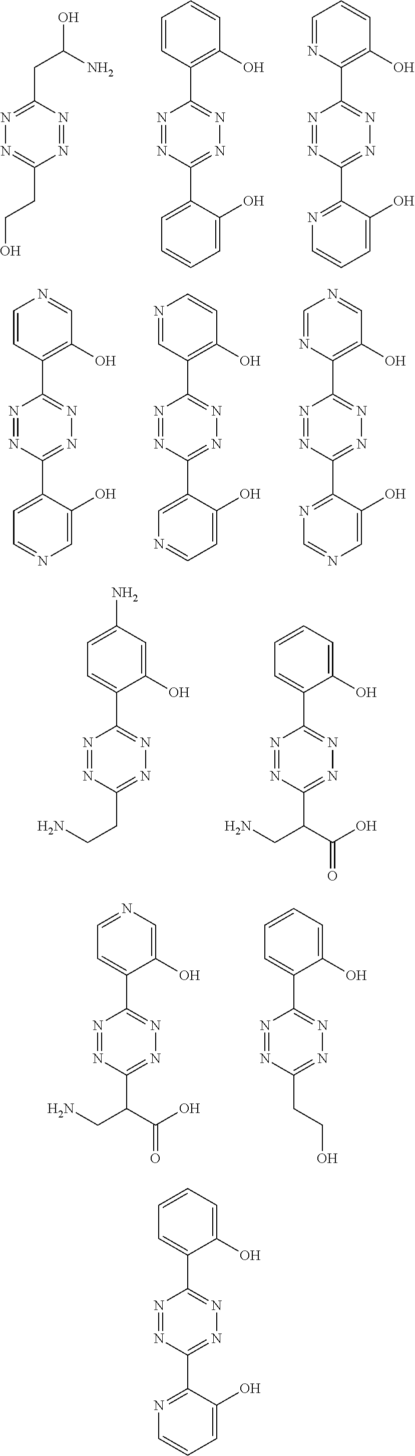

or a pharmaceutically acceptable salt thereof.

In some embodiments, the compound of Formula (II) is described, e.g., in Wilkovitsch, WO/2021/119268, WO/2022/072949, and/or Battisti et al., 2021, J Med Chem, 64, 15297-15312, which are incorporated herein by reference in their entirety.

In some embodiments the compound of Formula (II) may be optimized specifically for using as an antidote to a SENIT compound of Formula (A) or Formula (I). The click to release kinetics depend on the ability of Formula (II) at different c Log P and click rate to react with TCO-labeled affinity ligands of Formulae (A) and/or (I) in vivo. In some embodiments, the compound of Formula (II) is selected as an antidote such that the reaction rate constant is greater than about 50,000 M⁻¹s⁻¹ and the compounds is overall hydrophilic (e.g., strongly negative c Log P). Without being bound by any theory, the compound is able to achieve about or greater than about 99% conversion in about one hour in vivo. One example of a compound of Formula (II) in the present disclosure includes a hydrophilic tetrazine with a rate constant greater than about 30,000M⁻¹ sec⁻¹.

### Salts and Pharmaceutically Acceptable Salts

In some embodiments, a salt (such as a pharmaceutically acceptable salt) of a compound of this disclosure, such as Formula (A), Formula (I), or Formula (II) (or any other compounds disclosed in the present disclosure) is formed between an acid and a basic group of the compound, such as an amino functional group, or a base and an acidic group of the compound, such as a carboxyl functional group. According to another embodiment, the compound is an acid addition salt (e.g., a pharmaceutically acceptable addition salt).

In some embodiments, acids commonly employed to form pharmaceutically acceptable salts of the compounds of the present disclosure include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds of the present disclosure include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

### Compositions, Formulations, and Routes of Administration

The present application also provides pharmaceutical compositions comprising an effective amount of a compound of any one of the Formulae disclosed herein, or a pharmaceutically acceptable salt thereof, and a pharmaceutically acceptable carrier. The pharmaceutical composition may also comprise any one of the additional therapeutic agents described herein. In certain embodiments, the application also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

### Routes of Administration and Dosage Forms

The pharmaceutical compositions of the present application include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

### Dosages and Regimens

In the pharmaceutical compositions of the present application, a compound of the present disclosure is present in an effective amount (e.g., a therapeutically effective amount). Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician. For example, a compound of Formulae (A) or (I) can be administered at a dose that is effective for imaging.

In some embodiments, an effective amount of the compound can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg). In some embodiments, an effective amount of a compound is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

### Methods of Use

The present application further provides a method of imaging a cell or tissue, the method comprising:
(i) contacting the cell or tissue with a compound of Formula (I), or a pharmaceutically acceptable salt thereof, or a composition comprising same. and
(ii) waiting a sufficient amount of time to allow the affinity ligand Y¹ within the compound of Formula (I) to bind to its biological target within the cell or tissue.

In some embodiments, the contacting is carried out in vitro, in vivo, or ex vivo. In some embodiments, the method includes (i) administering a compound of Formula (I), or a pharmaceutically acceptable salt thereof (or a pharmaceutical composition comprising same), to a subject; and (ii) waiting a sufficient amount of time to allow the affinity ligand Y¹ within the compound of Formula (I) to bind to its biological target within the cell or tissue of the subject to be imaged (e.g., cancer tumor tissue). In some embodiments, the method includes diagnosing a disease in a subject. In some embodiments, the method includes monitoring treatment of a disease in a subject. In some embodiments, the method of diagnosis or monitoring includes observing a signal in step (iii) or step (iv) (e.g., observing a signal in an image obtained in step (iii) or step (iv) attributable to the radiotracer Y³ in the compound of Formula (I) is indicative of the presence of a disease biomarker to which the affinity ligand Y¹ binds, and, e.g., indicative of the disease to be diagnosed and/or indicative of the disease progression or regression, as the case may be). The method may also comprise comparing images obtained from subjects exhibiting the symptoms of the disease or condition with the images obtained from healthy subjects. In one example, overabundance of the disease biomarker in cells and/or tissues of the subject may be indicative of the disease such as cancer or any other disease described herein.

In some embodiments, the method includes a step of (iii) imaging the subject (or the cell and/or the tissue) with a suitable imaging technique. In some embodiments, the method includes a step of (iv) after (iii), of contacting the cell or the tissue with (or administering to the subject) a compound of Formula (II), or a salt thereof (e.g., a pharmaceutically acceptable salt thereof), or a composition comprising same (e.g., a pharmaceutical composition comprising same).

In some embodiments, the method includes a step of (iii) contacting the cell or the tissue with (or administering to the subject) a compound of Formula (II), or a salt thereof (e.g., a pharmaceutically acceptable salt thereof), or a composition comprising same (e.g., a pharmaceutical composition comprising same). In some embodiments, the method includes a step of (iv), after (iii), of imaging the subject (or the cell and/or the tissue) with a suitable imaging technique.

In some embodiments, contacting the cell, the component of the cell, or the tissue with a compound of Formula (II) results in a reaction (e.g., “click” reaction) of the compound of Formula (II) with a TCO moiety within the Formula (A) or Formula (I), leading to cleavage and molecular disassembly of the Formula (A) or Formula (I), thereby releasing the radiotracer Y³ (or a fluorophore R¹⁰) to circulation to be eliminated from the patient's body.

In some embodiments, the methods provided herein comprise waiting a time sufficient to allow the compound of Formula (A) or (I) to accumulate at a cell or tissue site (e.g., a cell or tissue site in a subject) associated with the disease, prior to imaging. In some embodiments, the methods provided herein comprise waiting a time sufficient to allow affinity ligand Y¹ within the compound of Formula (A) or (I) to bind its biological target at a cell or tissue site (e.g., a cell or tissue site in a subject) associated with the disease, prior to imaging. In some embodiments, the time sufficient is from about 30 seconds to about 24 hours, for example, about 30 seconds to about 24 hours, about 30 seconds to about 12 hours, about 30 seconds to about 6 hours, about 30 seconds to about 2 hours, about 30 seconds to about 1 hour, about 30 seconds to about 30 minutes, about 30 seconds to about 10 minutes, about 10 minutes to about 24 hours, about 10 minutes to about 12 hours, about 10 minutes to about 6 hours, about 10 minutes to about 2 hours, about 10 minutes to about 1 hour, about 10 minutes to about 30 minutes, about 30 minutes to about 24 hours, about 30 minutes to about 12 hours, about 30 minutes to about 6 hours, about 30 minutes to about 2 hours, about 30 minutes to about 1 hour, about 1 hour to about 24 hours, about 1 hour to about 12 hours, about 1 hour to about 6 hours, about 1 hour to about 2 hours, about 2 hours to about 24 hours, about 2 hours to about 12 hours, about 2 hours to about 6 hours, about 6 hours to about 24 hours, about 6 hours to about 12 hours, or about 12 hours to about 24 hours.

In some embodiments, the suitable imaging technique is a non-invasive imaging technique. In some embodiments, the suitable imaging technique is a minimally invasive imaging technique. As used herein, the term “minimally invasive imaging technique” comprises imaging techniques employing the use of an internal probe or injection of a compound or radiotracer via syringe. Example imaging techniques include, but are not limited to, fluoroscopic imaging, x-ray imaging, magnetic resonance imaging (MRI), ultrasound imaging, photoacoustic imaging, thermographic imaging, tomographic imaging, echocardiographic imaging, positron emission tomography (PET) imaging, PET with computed tomography (CT) imaging, PET-MRI, single-photon emission computed tomography (SPECT), and ultrasound imaging. In some embodiments, the suitable imaging technique is selected from the group consisting of PET imaging, PET-CT, PET-MRI, and SPECT. In some embodiments, the suitable imaging technique is selected from the group consisting of positron emission tomography (PET) imaging, positron emission tomography (PET) with computed tomography imaging, and positron emission tomography (PET) with magnetic resonance imaging (MRI). In some embodiments, the suitable imaging technique is selected positron emission tomography (PET) imaging.

Imaging techniques such as PET and SPECT have become an important clinical diagnostic and research modality, and also a valuable technology in drug discovery and development. PET offers picomolar sensitivity and is a fully translational technique that requires specific probes radiolabeled with a usually short-lived positron-emitting radionuclide. In one example, carbon-11 (radioactive half-life (t_{1/2})=20.4 min) and fluorine-18 (t_{1/2}=109.7 min) are commonly used radionuclides in PET imaging. PET has provided the capability of measuring biological processes at the molecular and metabolic levels in vivo by the detection of the photons formed as a result of the annihilation of the emitted positrons. SPECT is a nuclear imaging scan that needs a radioactive tracer. The tracer is what allows doctors to see how blood flows to tissues and organs. The radioisotopes typically used in SPECT are iodine-123, technetium-99m, xenon-133, thallium-201, and fluorine-18. These radioactive forms of natural elements pass through the body and can be detected by the appropriate scanner.

In some embodiments, the imaging technique of any of the imaging steps of the methods of the present disclosure is a fluorescence imaging, such as microscopy, imaging probes, and spectroscopy. The fluorescence imaging devices include an excitation source, the emitted light collection source, optionally optical filters, and a means for visualization (e.g., a digital camera for taking fluorescence imaging photographs). Suitable examples of fluorescence imaging include internal reflection fluorescence microscopy, light sheet fluorescence microscopy, and fluorescence-lifetime imaging microscopy. Suitable imaging techniques are described, for example, in Rao, J. et al., Fluorescence imaging in vivo: recent advances, Current Opinion in Biotechnology, 18, (1), 2007, 17-25, which is incorporated herein by reference in its entirety.

In some embodiments, the present disclosure provides a method of profiling a cell, the method comprising (i) obtaining the cell from a subject, and (ii) imaging the cell according to the methods described herein. In some embodiments, the present disclosure provides a method of examining a cell using a cytometry technique, the method comprising (i) obtaining the cell from a subject, and (ii) imaging the cell according to the method described herein. Suitable examples of cytometry techniques include image cytometry, holographic cytometry, Fourier ptychography cytometry, and fluorescence cytometry. In some embodiments, the present disclosure provides a method of diagnosing a disease or condition of a subject by examining pathology of a cell obtained from the subject, the method comprising (i) obtaining the cell from a subject, and (ii) imaging the cell according to the methods described herein. In some embodiments, the present disclosure provides a method of monitoring progression of disease or condition (or monitoring efficacy of treatment of disease or condition) of a subject by examining pathology of a cell obtained from the subject, the method comprising (i) obtaining the cell from the subject, and (ii) imaging the cell according to the method described herein. In some embodiments, the present disclosure provides a method of detecting a disease biomarker in a cell, the method comprising (i) obtaining the cell from a subject, and (ii) imaging the cell according to the method described herein. In some embodiments, the cell is obtained from the subject using image-guided biopsy, fine needle aspiration (FNA), surgical tissue harvesting, punch biopsy, liquid biopsy, brushing, swab, touch-prep, fluid aspiration or blood analysis. In some embodiments, the cell is obtained from the subject using fine needle aspiration (FNA). In some embodiments, the cell is selected from a cancer cell, an immune system cell, and a host cell (the methods of the present disclosure are useful for hepatocyte profiling in liver disease etc.). In some embodiments, the cell is a cancer cell.

The present application further provides a method of treating a disease or condition, the method comprising:

(i) administering to a subject in need thereof a therapeutically effective amount of a compound of Formula (I), or a pharmaceutically acceptable salt thereof, or a pharmaceutical composition comprising same.

In some embodiments, Y³ in the compound of Formula (I) is a toxic radioisotope (e.g., ⁴⁷Sc, ⁹⁰Y, ^{114m}In, ¹³¹I, ¹⁷⁷Lu, ¹⁸⁶Re, ¹⁸⁸Re, ²¹¹At, ²¹²Bi, ²¹²Pb, ²¹²Bi, ²¹³Bi, ²²³Ra, ²²⁵Ac, and ²²⁷Th). In some embodiments, Y¹ in the compound of Formula (I) is an affinity ligand to a biomarker of a disease to be treated (e.g., Y¹ is a cancer-targeting peptide or an antibody). In some embodiments, the method is a theranostic method, as described, e.g., in Martin et al., RadioGraphics, 40, 6, 2020, 1715-1740, which is incorporated herein in its entirety. For example, a radiotracer may be α, β, and γ-emitter wherein γ-rays are used for PET/SPECT imaging and α- and β-particle emissions are used for therapy (e.g., to kill cancer cells). In another example, o is 2 and p is 2, hence there are two Y²-Y³ groups in the compound of Formula (I), wherein one such a group can be used for diagnosing the disease (e.g., for locating a tumor in the subject by imaging the subject) and another such a group can be used for treating the disease, e.g., for radiation therapy of cancer. In some embodiments, the disease is thyroid cancer, neuroblastoma, prostate cancer, endocrine tumor, liver cancer, or lymphoma, or any of the cancers described herein.

In some embodiments, the method includes a step of:

(ii) waiting a sufficient amount of time to allow the affinity ligand Y^{L} within the compound of Formula (I) to bind to its biological target within the cell or tissue of the subject.

In some embodiments, the method includes a step of (iii) imaging the subject with a suitable imaging technique. In some embodiments, the method includes diagnosing the subject and/or monitoring treatment of the subject as described herein.

In some embodiments, the method includes a step of:

(iv) after (iii), administering to the subject a compound of Formula (II), or a pharmaceutically acceptable salt thereof, or pharmaceutical composition comprising same. In some embodiments, the method allows to avoid excessive exposure to radiation and concomitant side-effects, and/or allows to terminate the treatment on demand, e.g., when the treatment goal has been achieved.

In some embodiments, the method includes a step of:

(iii) administering to the subject a compound of Formula (II), or a pharmaceutically acceptable salt thereof, or a pharmaceutical composition comprising same. In some embodiments, the method includes a step of:

(iv), after (iii), imaging the subject with a suitable imaging technique. In some embodiments, the method includes diagnosing the subject and/or monitoring treatment of the subject as described herein.

In some embodiments, a disease as described herein is selected from the group consisting of an autoimmune disorder, an inflammatory disorder, a skin disorder, cancer, and a cardiovascular disorder.

In some embodiments, the cancer is selected from sarcoma, angiosarcoma, fibrosarcoma, rhabdomyosarcoma, liposarcoma, myxoma, rhabdomyoma, fibroma, lipoma, teratoma, lung cancer, bronchogenic carcinoma squamous cell, undifferentiated small cell, undifferentiated large cell, adenocarcinoma, alveolar bronchiolar carcinoma, bronchial adenoma, sarcoma, lymphoma, chondromatous hamartoma, mesothelioma, gastrointestinal cancer, cancer of the esophagus, squamous cell carcinoma, adenocarcinoma, leiomyosarcoma, lymphoma, cancer of the stomach, carcinoma, lymphoma, leiomyosarcoma, cancer of the pancreas, ductal adenocarcinoma, insulinoma, glucagonoma, gastrinoma, carcinoid tumor, vipoma, cancer of the small bowel, adenocarcinoma, lymphoma, carcinoid tumors, Kaposi's sarcoma, leiomyoma, hemangioma, lipoma, neurofibroma, fibroma, cancer of the large bowel or colon, tubular adenoma, villous adenoma, hamartoma, leiomyoma, genitourinary tract cancer, cancer of the kidney adenocarcinoma, Wilm's tumor (nephroblastoma), lymphoma, leukemia, cancer of the bladder, cancer of the urethra, squamous cell carcinoma, transitional cell carcinoma, cancer of the prostate, cancer of the testis, seminoma, teratoma, embryonal carcinoma, teratocarcinoma, choriocarcinoma, sarcoma, interstitial cell carcinoma, fibroma, fibroadenoma, adenomatoid tumors, lipoma, liver cancer, hepatoma hepatocellular carcinoma, cholangiocarcinoma, hepatoblastoma, angiosarcoma, hepatocellular adenoma, hemangioma, bone cancer, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor, chordoma, osteochrondroma (osteocartilaginous exostoses), benign chondroma, chondroblastoma, chondromyxofibroma, osteoid osteoma giant cell tumor, nervous system cancer, cancer of the skull, osteoma, hemangioma, granuloma, xanthoma, osteitis deformans, cancer of the meninges meningioma, meningiosarcoma, gliomatosis, cancer of the brain, astrocytoma, medulloblastoma, glioma, ependymoma, germinoma (pinealoma), glioblastoma multiforme, oligodendroglioma, schwannoma, retinoblastoma, congenital tumors, cancer of the spinal cord, neurofibroma, meningioma, glioma, sarcoma, gynecological cancer, cancer of the uterus, endometrial carcinoma, cancer of the cervix, cervical cancer, cervical carcinoma, pre tumor cervical dysplasia, cancer of the ovaries, ovarian carcinoma, serous cystadenocarcinoma, mucinous cystadenocarcinoma, unclassified carcinoma, granulosa-theca cell tumor, Sertoli Leydig cell tumor, dysgerminoma, malignant teratoma, cancer of the vulva, squamous cell carcinoma, intraepithelial carcinoma, adenocarcinoma, fibrosarcoma, melanoma, cancer of the vagina, clear cell carcinoma, squamous cell carcinoma, botryoid sarcoma, embryonal rhabdomyosarcoma, cancer of the fallopian tubes, hematologic cancer, cancer of the blood, acute myeloid leukemia (AML), chronic myeloid leukemia (CML), acute lymphoblastic leukemia (ALL), chronic lymphoblastic leukemia, chronic lymphocytic leukemia, myeloproliferative diseases, multiple myeloma, myelodysplastic syndrome, Hodgkin's lymphoma, non-Hodgkin's lymphoma (malignant lymphoma), Waldenstrom's macroglobulinemia, skin cancer, malignant melanoma, basal cell carcinoma, squamous cell carcinoma, Kaposi's sarcoma, moles dysplastic nevi, lipoma, angioma, dermatofibroma, keloids, psoriasis, adrenal gland cancer, and neuroblastoma.

In some embodiments, the disease is selected from the group consisting of graft-versus-host disease, rheumatoid arthritis, systemic lupus erythematosus, Hashimoto's thyroiditis, multiple sclerosis, myasthenia gravis, type I diabetes, uveitis, posterior uveitis, allergic encephalomyelitis, glomerulonephritis, rheumatic fever, post-infectious glomerulonephritis, psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne, alopecia areata, keratoconjunctivitis, vernal conjunctivitis, uveitis associated with Behcet's disease, keratitis, herpetic keratitis, conical cornea, dystrophia epithelialis corneae, corneal leukoma, ocular pemphigus, Mooren's ulcer, scleritis, Graves' opthalmopathy, Vogt-Koyanagi-Harada syndrome, sarcoidosis, pollen allergies, reversible obstructive airway disease, bronchial asthma, allergic asthma, intrinsic asthma, extrinsic asthma, dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns, coeliac diseases, proctitis, eosinophilic gastroenteritis, mastocytosis, Crohn's disease, ulcerative colitis, migraine, rhinitis, eczema, interstitial nephritis, Goodpasture's syndrome, hemolytic-uremic syndrome, diabetic nephropathy, multiple myositis, Guillain-Barre syndrome, Meniere's disease, polyneuritis, multiple neuritis, mononeuritis, radiculopathy, hyperthyroidism, Basedow's disease, pure red cell aplasia, aplastic anemia, hypoplastic anemia, idiopathic thrombocytopenic purpura, autoimmune hemolytic anemia, agranulocytosis, pernicious anemia, megaloblastic anemia, anerythroplasia, osteoporosis, sarcoidosis, fibroid lung, idiopathic interstitial pneumonia, dermatomyositis, leukoderma vulgaris, ichthyosis vulgaris, photoallergic sensitivity, cutaneous T cell lymphoma, arteriosclerosis, atherosclerosis, aortitis syndrome, polyarteritis nodosa, myocardosis, scleroderma, Wegener's granuloma, Sjogren's syndrome, adiposis, eosinophilic fascitis, lesions of gingiva, periodontium, alveolar bone, substantia ossea dentis, glomerulonephritis, male pattern alopecia, alopecia senilis by preventing epilation, alopecia senilis by providing hair germination and/or promoting hair generation and hair growth, muscular dystrophy, pyoderma, Sezary's syndrome, Addison's disease, ischemia-reperfusion injury of organs, transplantation disease, ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, cataracta, siderosis, retinitis pigmentosa, senile macular degeneration, vitreal scarring, corneal alkali burn, dermatitis erythema multiforme, linear IgA ballous dermatitis and cement dermatitis, gingivitis, periodontitis, sepsis, pancreatitis, aging, carcinogenesis, metastasis of carcinoma and hypobaropathy, histamine or leukotriene-C4 release associated diseases, Behcet's disease, autoimmune hepatitis, primary biliary cirrhosis, sclerosing cholangitis, partial liver resection, acute liver necrosis, necrosis caused by toxin, viral hepatitis, shock, anoxia, B-virus hepatitis, non-A/non-B hepatitis, cirrhosis, alcoholic cirrhosis, hepatic failure, fulminant hepatic failure, late-onset hepatic failure, acute-on-chronic liver failure, cytomegalovirus infection, HCMV infection, AIDS, senile dementia, trauma, chronic bacterial infection, malignancy of lymphoid origin, acute lymphocytic leukemia, chronic lymphocytic leukemia, acute lymphocytic lymphoma, and chronic lymphocytic lymphoma.

In some embodiments, the disease is selected from the group consisting of systemic lupus erythematosis, chronic rheumatoid arthritis, type I diabetes mellitus, inflammatory bowel disease, biliary cirrhosis, uveitis, multiple sclerosis, Crohn's disease, ulcerative colitis, bullous pemphigoid, sarcoidosis, psoriasis, autoimmune myositis, Wegener's granulomatosis, ichthyosis, Graves ophthalmopathy, asthma, schleroderma and Sjogren's syndrome.

In some embodiments, any of the treatment methods may include co-administering to the subject an additional therapeutic agent as described herein. Likewise, any of the methods for monitoring progression of treatment may refer to treatment with any of the additional therapeutic agents as described herein, or an experimental drug substance.

In some embodiments, the therapeutic agent is an antibody. Example antibodies for use in combination therapy include but are not limited to trastuzumab (e.g. anti-HER2), ranibizumab (e.g. anti-VEGF-A), bevacizumab (e.g. anti-VEGF), panitumumab (e.g. anti-EGFR), cetuximab (e.g. anti-EGFR), rituxan (anti-CD20), antibodies directed to c-MET, and antibody inhibitors of granzyme B (e.g., Clone GB 11, Clone GrB-7, and NCL-L-Gran-B), ipilimumab (anti-CTLA-4), nivolumab (anti-PD-1), pembrolizumab (anti-PD-1), atezolizumab (anti-PD-1), elotuzumab (anti-SLAM7), and daratumumab (anti-CD38).

In some embodiments, the therapeutic agent is a steroid. Example steroids include corticosteroids such as cortisone, dexamethasone, hydrocortisone, methylprednisolone, prednisolone, and prednisone. In some embodiments, the additional agent is a corticosteroid.

In some embodiments, the therapeutic agent is an anti-inflammatory compound. Example anti-inflammatory compounds include aspirin, choline salicylates, celecoxib, diclofenac potassium, diclofenac sodium, diclofenac sodium with misoprostol, diflunisal, etodolac, fenoprofen, flurbiprofen, ibuprofen, ketoprofen, meclofenamate sodium, mefenamic acid, nabumetone, naproxen, naproxen sodium, oxaprozin, piroxican, rofecoxib, salsalate, sodium salicylate, sulindac, tolmetin sodium, and valdecoxib.

In some embodiments, the therapeutic agent is chemotherapeutic agent. Example chemotherapeutic agents include, but are not limited to, a cytostatic agent, cisplatin, doxorubicin, taxol, etoposide, irinotecan, topotecan, paclitaxel, docetaxel, epothilones, tamoxifen, 5-fluorouracil, methotrexate, temozolomide, cyclophosphamide, SCH 66336, R115777, L778,123, BMS 214662, gefitinib, erlotinib hydrochloride, antibodies to EGFR, imatinib mesylate, intron, ara-C, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, oxaliplatin, folinic acid, pentostatin, vinblastine, vincristine, vindesine, bleomycin, dactinomycin, daunorubicin, doxorubicin, epirubicin, idarubicin, mithramycin, deoxycoformycin, mitomycin-C, L-asparaginase, teniposide, 17α-ethinylestradiol, diethylstilbestrol, testosterone, prednisone, fluoxymesterone, dromostanolone propionate, testolactone, megestrol acetate, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, carboplatin, hydroxyurea, amsacrine, procarbazine, mitotane, mitoxantrone, levamisole, vinorelbine, anastrazole, letrozole, capecitabine, reloxafine, hexamethylmelamine, bevacizumab, bexxar, velcade, zevalin, trisenox, xeloda, vinorelbine, porfimer, erbitux, liposomal, thiotepa, altretamine, melphalan, trastuzumab, fulvestrant, exemestane, ifosfamide, rituximab, C225, alemtuzumab, clofarabine, cladribine, aphidicolin, sunitinib, dasatinib, tezacitabine, Sml1, triapine, didox, trimidox, amidox, 3-AP, MDL-101,731, bendamustine, ofatumumab, and GS-1101 (also known as CAL-101).

In some embodiments, the chemotherapeutic agent is selected from the group consisting of an alkylating agent (e.g., busulfan, chlorambucil, cisplatin, cyclophosphamide (cytoxan), dacarbazine, ifosfamide, mechlorethamine (mustargen), and melphalan), a nitrosourea (e.g., carmustine, lomustine, semustine, and streptozocin), a triazine (e.g., dacarbazine) an anti-metabolite (e.g., 5-fluorouracil (5-FU), cytarabine (Ara-C), fludarabine, gemcitabine, and methotrexate), a purine analog (e.g., 6-mercaptopurine, 6-thioguanine, and pentostatin (2-deoxycoformycin)), a mitotic inhibitor (e.g., docetaxel, etoposide (VP16), teniposide, paclitaxel, taxol, vinblastine, vincristine, and vinorelbine), an anti-tumor antibiotic (e.g., bleomycin, dactinomycin, daunorubicin, doxorubicin, mitomycin, plicamycin, and idarubicin), a platinum chemotherapeutic agent (e.g., cisplatin and carboplatin), an anthracenedione (e.g., mitoxantrone), a toxin (e.g., ricin A-chain (Burbage, Leukemia research, 21.7 (1997): 681-690), diphtheria toxin A (Massuda et al., Proceedings of the National Academy of Sciences, 94.26 (1997): 14701-14706; Lidor, American journal of obstetrics and gynecology, 177.3 (1997): 579-585), pertussis toxin A subunit, E. colienterotoxin toxin A subunit, cholera toxin A subunit and Pseudomonas toxin c-terminal), and a gene therapy vector (e.g., a signal transducing protein (e.g., Src, Abl, and Ras), Jun, Fos, and Myc).

In some embodiments, the therapeutic agent is an immunotherapeutic agent. An immunotherapeutic agent generally triggers immune effector cells and molecules to target and destroy cells (e.g., cancer cells). The immune effector may be, for example, an antibody specific for a marker on the surface of a cell (e.g. a tumor cell). The antibody alone may serve as an effector of therapy or it may recruit other cells to effect cell killing. Various effector cells include, but are not limited to, cytotoxic T cells and NK cells.

Example immunotherapeutic agents include, but are not limited to, azathioprine, chlorambucil, cyclophosphamide, cyclosporine, daclizumab, infliximab, methotrexate, tacrolimus, immune stimulators (e.g., IL-2, IL-4, IL-12, GM-CSF, tumor necrosis factor; interferons alpha, beta, and gamma; F42K and other cytokine analogs; a chemokine such as MIP-1, MIP-1β, MCP-1, RANTES, IL-8; or a growth factor such FLT3 ligand), an antigenic peptide, polypeptide or protein, or an autologous or allogenic tumor cell composition (see e.g., Ravindranath & Morton, International reviews of immunology, 7.4 (1991): 303-329), hormonal therapy, adrenocorticosteroids, progestins (e.g., hydroxyprogesterone caproate, medroxyprogesterone acetate, and megestrol acetate), estrogens (e.g., diethylstilbestrol and ethinyl estradiol), anti-estrogens (e.g., testosterone propionate and fluoxymesterone), anti-androgens (e.g., flutamide), and gonadotropin-releasing hormone analogs (e.g., leuprolide). Additional immunotherapeutic agents are known in the art, and can be found, for example, in Rosenberg et al, New England Journal of Medicine, 319.25 (1988): 1676-1680; and Rosenberg et al, Annals of surgery, 210.4 (1989): 474).

### Definitions

As used herein, the term “about” means “approximately” (e.g., plus or minus approximately 10% of the indicated value).

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “aromatic” refers to a carbocycle or heterocycle having one or more polyunsaturated rings having aromatic character (i.e., having (4n+2) delocalized π (pi) electrons where n is an integer).

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1,-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “amino” refers to a group of formula —NH₂.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “Cₙ₋ₘ alkoxycarbonyl” refers to a group of formula —C(O)O-alkyl, wherein the alkyl group has n to m carbon atoms. In some embodiments, the alkyl group has 1 to 6, 1 to 4, or 1 to 3 carbon atoms. Examples of alkoxycarbonyl groups include, but are not limited to, methoxycarbonyl, ethoxycarbonyl, propoxycarbonyl (e.g., n-propoxycarbonyl and isopropoxycarbonyl), butoxycarbonyl (e.g., n-butoxycarbonyl and tert-butoxycarbonyl), and the like.

As used herein, the term “Cₙ₋ₘ alkylcarbonyl” refers to a group of formula —C(O)-alkyl, wherein the alkyl group has n to m carbon atoms. In some embodiments, the alkyl group has 1 to 6, 1 to 4, or 1 to 3 carbon atoms. Examples of alkylcarbonyl groups include, but are not limited to, methylcarbonyl, ethylcarbonyl, propylcarbonyl (e.g., n-propylcarbonyl and isopropylcarbonyl), butylcarbonyl (e.g., n-butylcarbonyl and tert-butylcarbonyl), and the like.

As used herein, the term “Cₙ₋ₘ alkylcarbonylamino” refers to a group of formula —NHC(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonylamino” refers to a group of formula —NHS(O)₂-alkyl, wherein the alkyl group has n to m carbon atoms. In some embodiments, the alkyl group has 1 to 6, 1 to 4, or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonyl” refers to a group of formula —S(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminosulfonylamino” refers to a group of formula —NHS(O)₂NH₂.

As used herein, the term “Cₙ₋ₘ alkylaminosulfonylamino” refers to a group of formula —NHS(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonylamino” refers to a group of formula —NHS(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminocarbonylamino”, employed alone or in combination with other terms, refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group has, independently, 1 to 6, 1 to 4, or 1 to 3 carbon atoms.

As used herein, the term “carbamyl” to a group of formula —C(O)NH₂.

As used herein, the term “Cₙ₋ₘ alkylcarbamyl” refers to a group of formula —C(O)—NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “thio” refers to a group of formula —SH.

As used herein, the term “Cₙ₋ₘ alkylthio” refers to a group of formula —S-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfinyl” refers to a group of formula —S(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

As used herein, the term “carbonyl”, employed alone or in combination with other terms, refers to a —C(═O)— group, which may also be written as C(O).

As used herein, the term “carboxy” refers to a —C(O)OH group.

As used herein, the term “cyano-C₁₋₃ alkyl” refers to a group of formula —(C₁₋₃ alkylene)-CN.

As used herein, the term “HO—C₁₋₃ alkyl” refers to a group of formula —(C₁₋₃ alkylene)-OH.

As used herein, “halo” refers to F, Cl, Br, or I. In some embodiments, a halo is F, C₁, or Br.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to 10 carbon atoms. In some embodiments, the aryl group is phenyl or naphtyl.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Ring-forming carbon atoms of a cycloalkyl group can be optionally substituted by 1 or 2 independently selected oxo or sulfide groups (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (C₃₋₁₀). In some embodiments, the cycloalkyl is a C₃₋₁₀ monocyclic or bicyclic cyclocalkyl. In some embodiments, the cycloalkyl is a C₃₋₇ monocyclic cyclocalkyl. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, cycloalkyl is cyclopropyl, cyclobutyl, cyclopentyl, or cyclohexyl.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl is a 5-10 membered monocyclic or bicyclic heteroaryl having 1, 2, 3 or 4 heteroatom ring members independently selected from nitrogen, sulfur and oxygen. In some embodiments, the heteroaryl is a 5-6 monocyclic heteroaryl having 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, 7-, 8-, 9- or 10-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by 1 or 2 independently selected oxo or sulfido groups (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, the term “oxo” refers to an oxygen atom as a divalent substituent, forming a carbonyl group when attached to a carbon (e.g., C═O), or attached to a heteroatom forming a sulfoxide or sulfone group.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” a cell with a compound of the invention includes the administration of a compound of the present invention to an individual or patient, such as a human, having the cell, as well as, for example, introducing a compound of the invention into a sample containing a cellular or purified preparation.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

As used herein, the term “radioisotope” refers to an atom having an atomic mass or mass number different from the atomic mass or mass number typically found in nature (i.e., naturally occurring).

As used herein, the term “isotopic enrichment factor” refers to the ratio between the isotopic abundance and the natural abundance of a specified isotope. The following radioisotopes are defined by way of example (any of the isotopes disclosed herein can be defined in a similar manner).

“¹⁸F” refers to the radioisotope of fluorine having 9 protons and 9 neutrons. “F” refers to the stable isotope of fluorine having 9 protons and 10 neutrons (i.e., the “¹⁹F isotope”). A compound of the present disclosure has an isotopic enrichment factor for each designated ¹⁸F atom of at least 3500 (52.5% ¹⁸F incorporation at each designated ¹⁸F atom), at least 4000 (60% ¹⁸F incorporation), at least 4500 (67.5% ¹⁸F incorporation), at least 5000 (75% ¹⁸F), at least 5500 (82.5% ¹⁸F incorporation), at least 6000 (90% ¹⁸F incorporation), at least 6333.3 (95% ¹⁸F incorporation), at least 6466.7 (97% ¹⁸F incorporation), at least 6600 (99% ¹⁸F incorporation), or at least 6633.3 (99.5% ¹⁸F incorporation).

“¹¹C” refers to the radioisotope of carbon having 6 protons and 5 neutrons. “C” refers to the stable isotope of carbon having 6 protons and 6 neutrons (i.e., the “¹²C isotope”). A compound of the present disclosure has an isotopic enrichment factor for each designated ¹¹C atom of at least 3500 (52.5% ¹¹C incorporation at each designated ¹¹C atom), at least 4000 (60% ¹¹C incorporation), at least 4500 (67.5% ¹¹C incorporation), at least 5000 (75% ¹¹C), at least 5500 (82.5% ¹¹C incorporation), at least 6000 (90% ¹¹C incorporation), at least 6333.3 (95% ¹¹C incorporation), at least 6466.7 (97% ¹¹C incorporation), at least 6600 (99% ¹¹C incorporation), or at least 6633.3 (99.5% ¹¹C incorporation).

“¹²³I” refers to the radioisotope of iodine having 53 protons and 70 neutrons. “¹²⁵I” refers to the radioisotope of iodine having 53 protons and 72 neutrons. “¹³¹I” refers to the radioisotope of iodine having 53 protons and 78 neutrons. “I” refers to any abundant, stable isotope or a combination of stable, non-radioactive isotopes of iodine (e.g., the “¹²⁷I isotope”). A compound of the present disclosure has an isotopic enrichment factor for each designated ^{123/125/131}I atom of at least 3500 (52.5% incorporation at each designated ^{123/125/131}I atom), at least 4000 (60% incorporation), at least 4500 (67.5% incorporation), at least 5000 (75% incorporation), at least 5500 (82.5% incorporation), at least 6000 (90% incorporation), at least 6333.3 (95% incorporation), at least 6466.7 (97% incorporation), at least 6600 (99% incorporation), or at least 6633.3 (99.5% incorporation).

### EXAMPLES

Materials. Solvents, reagents, and enzymes were purchased from Sigma-Aldrich (St. Louis, MO) and used without further purification, unless otherwise stated. Aqueous solution was prepared using MilliQ water (Millipore). AlexaFluor647-NHS and DOTA-NHS were obtained from Click Chemistry Tools (Scottsdale, AZ) and Macrocyclics, Inc. (Plano, TX), respectively. ⁶⁴CuCl₂ was purchased from the Department of Medical Physics in the University of Wisconsin (Madison, WI). Antibody (trastuzumab) was generously provided by Genentech (South San Francisco, CA).

Cell lines and animal models. HT1080 human epithelial cancer cells derived from a fibrosarcoma (ATCC) overexpressing HER2 fused to GFP at the c-terminus (HT-HER2-GFP) were prepared via transfection with plasmid carrying HER2-GFP gene (Addegene; Watertown, MA; plasmid #39321) using Lipofectamine 3000 (Invitrogent) according to manufacturer protocols (HT-HER2-GFP). All animal research was performed in accordance with guidelines from the Institutional Subcommittee on Research Animal Care. Previous imaging guided experimental sample sizes in this study. HT-HER2-GFP tumors were generated by injecting 1 million cells intradermally in the flanks of 7-12 week old female nu/nu mice (MGB Cox 7 Core). For all procedures, mice were anesthetized with an isoflurane vaporizer on a heated stage; euthanasia was performed by CO₂ chamber when necessary, and all treatment groups underwent procedures and monitoring consecutively on the same day when possible, but in a randomized order.

Intravital imaging. All confocal images were collected using a customized Olympus FV1000 confocal microscope (Olympus America). A 2× (XLFluor, NA 0.14), a 4× (UPlanSApo, NA 0.16), and an XLUMPlanFL N 20× (NA 1.0) water immersion objective were used for imaging (Olympus America). Hoechst nuclear staining, HT-HER2-GFP tumor cells, Dextran-AF555 (Thermo), and SAFE647 were excited sequentially using a 405 nm, a 473 nm, a 559 nm, and a 633 nm diode laser, respectively, in combination with a DM-405/488/559/635 nm dichroic beam splitter. Emitted light was further separated by beam splitters (SDM-473, SDM-560, and SDM-640) and emission filters BA430-455, BA490-540, BA575-620, and BA655-755 (Olympus America). Confocal laser power settings were carefully optimized to avoid photobleaching, phototoxicity, or damage to the brain. All images were processed using Fiji (ImageJ2, Vers.2.3/1.53f). Blood half-life (t_{1/2}) measurement of the labeled antibody was performed using confocal imaging. Nu/nu mice (MGB Cox 7 Core) were anesthetized using isoflurane, stabilized using a stereotaxic (Kopf, Tujunga, CA) for motion-free imaging, and a vascular probe was injected to select areas in the ear with good vasculature for imaging. Time-series of confocal imaging stacks in multiple locations were initiated before injection of fluorescent Trastuzumab-SAFE647 (0.15 mg) via tail vein catheter. After 15 minutes, HK-Tz (0.1 mg in 100 uL PBS) was injected while Z-stacks from the area were collected every 6 minutes over two hours. Average fluorescence signal intensity was quantified using six ROIs, each inside the vasculature and outside of the vasculature (background) using Fiji (ImageJ2, Vers.2.3/1.53f). Background fluorescence was subtracted from the average signal inside the vasculature, and the values were analyzed and plotted in GraphPad Prism (San Diego, CA, Version 9.3.1 for Mac).

PET imaging. The PET-CT imaging procedure was similar to previously described methods. PET-CT imaging was performed 24 h after tail-vein injection of antibody (9.6±2.4 MBq/230±64.3 μCi in 150±10 μL). High-resolution contrast enhanced vascular CT (Inveon, Siemens, Munich, Germany) was conducted prior to the PET scan. The CT scan was acquired with X-ray power of 80 kVp and 500 μA, an exposure time of 370 to 400 ms, and an isotropic resolution of 90 μm. The ordered subsets expectation maximization (OSEM) and filtered back projection (FBP) algorithms were used for PET imaging reconstruction to obtain a spatial resolution approaching approximately 1 mm. For quantitative PET analysis, regions of interest were defined on the basis of anatomic CT data. The harvested lungs were placed in a glass chamber made in-house after ex vivo scintillation counting. For autoradiography, lungs were exposed to a storage phosphor screen in a cassette (GE Healthcare) for about 12 h and visualized using a Typhoon 9410 scanner (Amersham Biosciences).

Statistics. Unless otherwise indicated, results are expressed as mean±SEM throughout. Statistical analyses were performed using Prism (GraphPad), MATLAB (Mathworks), and Excel (Microsoft). Two-tailed tests, ANOVA tests, and spearman correlation tests were used with false-positive thresholds of α=0.05.

### Example 1—Fluorescent Probe

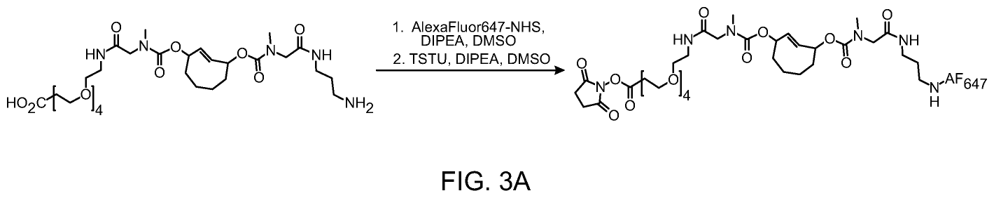

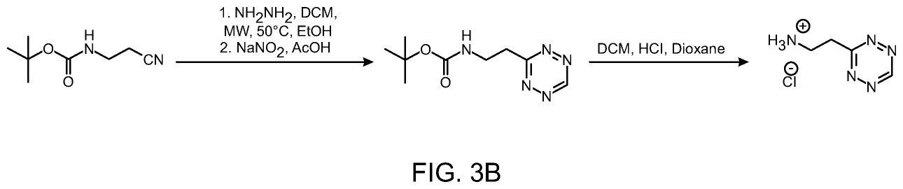

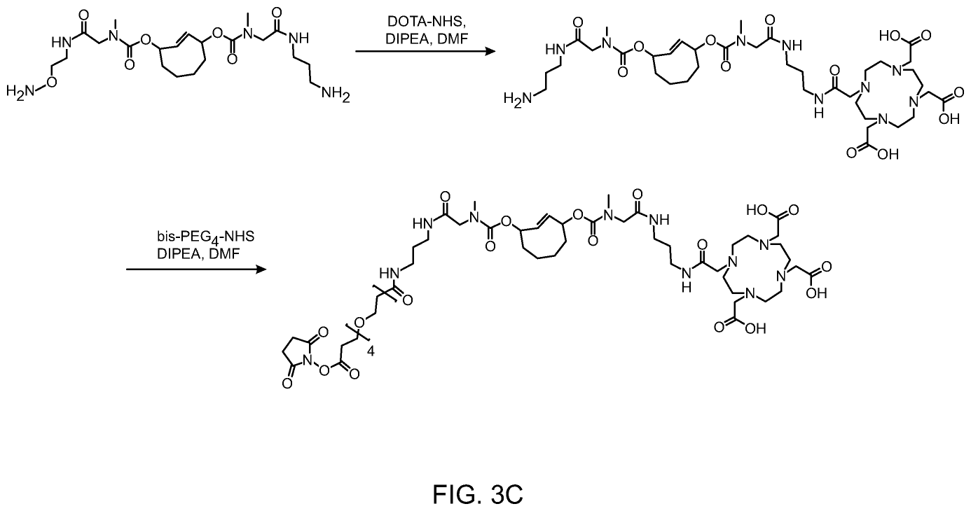

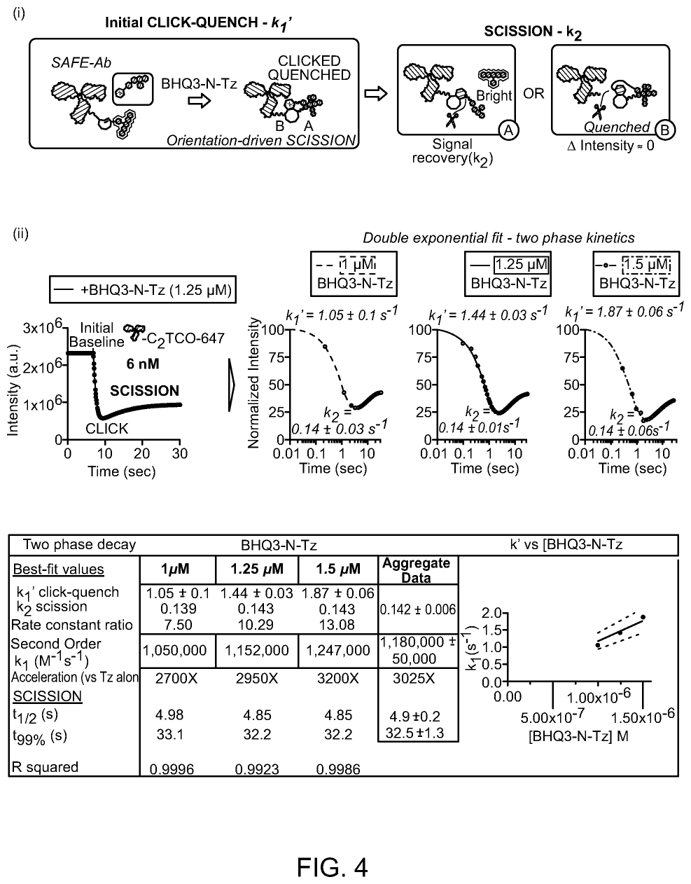

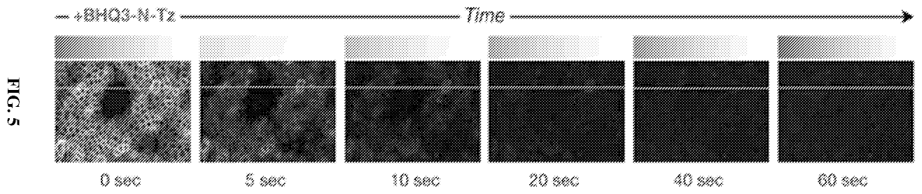

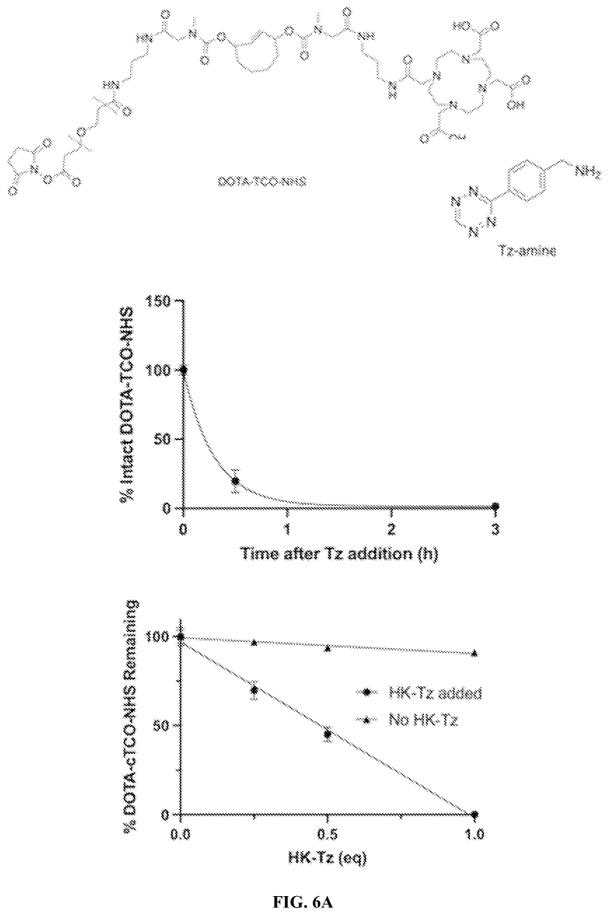

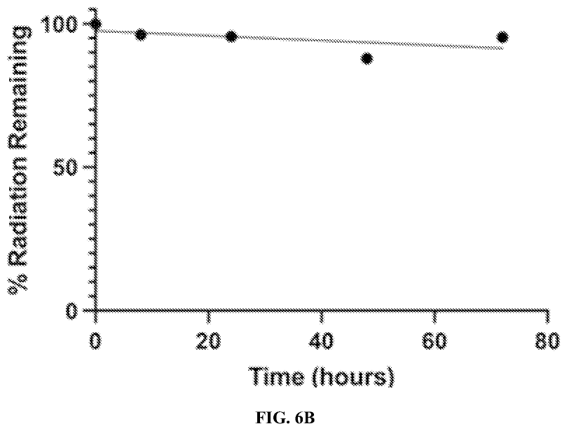

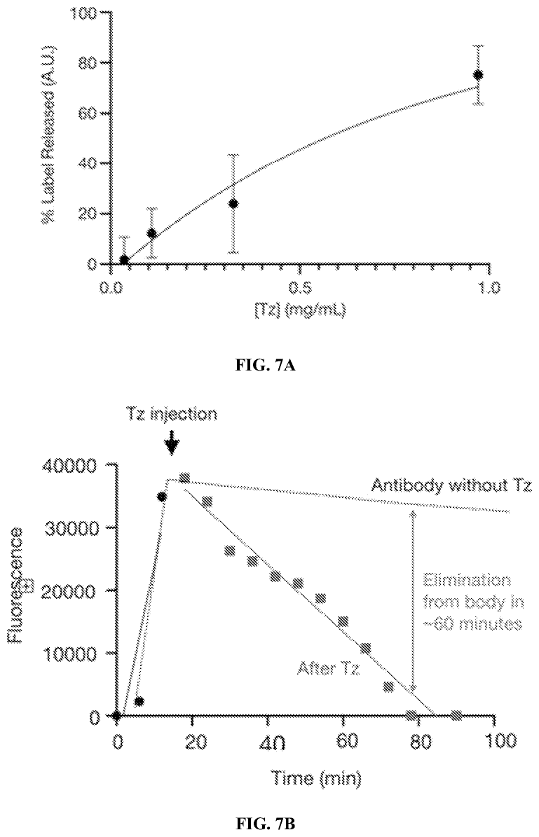

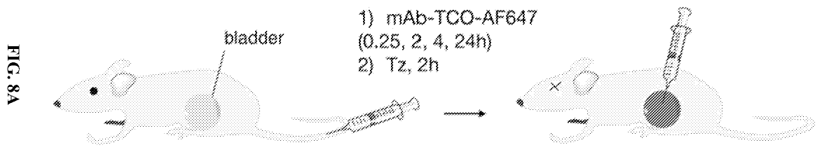

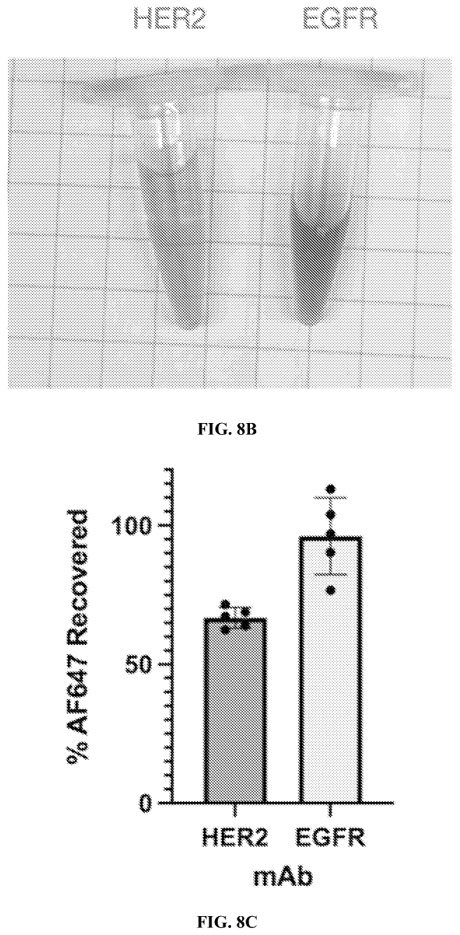

SAFE-647 was synthesized as described previously in Ko and shown in FIG. 3A. Briefly, HO₂C-TCO-NH₂ (1) was dissolved in anhydrous DMSO and reacted with AlexaFluor647-amine and DIPEA for 30 minutes at room temperature. The product was then activated via addition of TSTU (1 eq) and DIPEA (1 eq). After shaking vigorously for one minute, this mixture was analyzed via LCMS and determined to match the desired product. MS (+ESI) m/z: calculated for C₇₁H₁₀₃N₈O₂₇S₄⁺[M+H]⁺ 1627.58 found 1627.97.

### Example 2—DOTA Probe

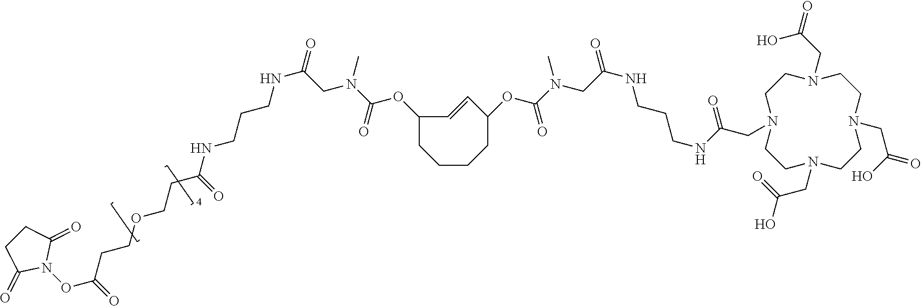

DOTA-TCO-NHS was synthesized by dissolving TCO-diamine (10 mg, 20.6 μmol) in anhydrous DMF (1 mL) and adding NHS-DOTA (11.4 mg, 15.0 μmol) and DIPEA (5 μL, 28.7 μmol). This mixture was shaken at room temperature for 1 hour, then bisNHS-PEG₄ (22 mg, 45.0 μmol) and DIPEA (5 μL, 28.7 μmol) were added and the mixture shaken for a further hour. The crude mixture was then loaded directly onto a reverse-phase column and purified using a gradient of 5-95% acetonitrile in water (0.1% formic acid). Fractions containing the product were identified via LC-MS, then combined and evaporated to provide the product as a colorless semisolid (15.1 mg, 59% yield). ¹H NMR (400 MHz, CDCl₃): δ 12.14 (brs, 3H), 8.13 (s, 1H), 7.95 (s, 1H), 7.82 (2H), 5.78 (t, J=27.9 Hz, 1H), 5.25 (t, J=20.3 Hz, 1H), 3.95-3.87 (m, 1H), 3.83 (t, J=6.8 Hz, 1H), 3.71 (t, J=6.0 Hz, 4H), 3.59 (t, J=6.3 Hz, 6H), 3.55-3.51 (m, 8H), 3.50-3.45 (m, 24H), 3.15-3.00 (m, 6H), 2.92 (t, J=5.82 Hz, 6H), 2.86 (t, J=5.7 Hz, 2H), 2.81 (s, 8H), 2.59 (s, 1H), 2.44 (t, J=6.3 Hz, 4H), 2.29 (t, J=6.5 Hz, 2H), 2.00-1.86 (m, 1H), 1.58-1.48 (m, 4H), 1.10-0.94 (m, 1H). ¹³C NMR (101 MHz, CDCl₃): δ 184.3, 172.8, 172.7, 170.2, 168.6, 167.4, 162.3, 155.1, 154.7, 129.4, 73.7, 69.8, 69.7, 69.6, 69.5, 66.9, 66.2, 65.2, 53.3, 41.7, 40.4, 38.3, 36.2, 35.8, 34.8, 31.6, 30.8, 29.3, 25.5, 25.2, 23.3, 17.6, 12.7. MS (+ESI) m/z: calculated for C₅₄H₉₀N₁₁O₂₂⁺[M+H]⁺ 1244.63 found 1244.90.

### Example 3—Antibody Conjugation

Conjugation of the probes prepared in Examples 1 and 2 was performed by preparing either trastuzumab or cetuximab in PBS (pH 7.4, 2 mg/mL). To these solutions was added a solution containing 8 eq of either SAFE-647 or SENIT in dry DMF (2 mM) and saturated sodium bicarbonate to achieve a final pH of about 8.5. These solutions were shaken at room temperature for 1 hour, then any remaining probe was removed by spin filtration (50 kDa MWCO, 7000 rcf for 5 min, ×5 in PBS). The solutions were then diluted to a concentration of 2 mg/mL in PBS and stored at 4° C. until further use (final DOL about 5.5).

### Example 4—⁶⁴Cu Labeling of Antibody

Metal free buffer solutions were prepared using Chelex® 100 Chelating Resin (100-200 mesh, BioRad). SENIT-trastuzumab (50 μg) was diluted with citrate buffer (400 μL, 0.1 M, pH 5.0). about 25 mCi (˜925 MBq) of ⁶⁴CuCl₂ in 0.1 N HCl was mixed with citrate buffer (0.1 M, pH 7.0) to form ⁶⁴Cu(OAc)₂ and pH was adjusted to ˜5. The antibody was labeled with ⁶⁴Cu at 50° C. for 30 min on a thermomixer (900 rpm). The labeling efficiency was monitored by iTLC showing >99% labeling with a specific activity of 19.2 MBq (514 μCi) ⁶⁴Cu/μg antibody. Trace amounts of unchelated ⁶⁴Cu were removed by EDTA chelation (final concentration about 5 mM) followed by centrifugation (MWCO 50 kDa) at 10 k×g for 5 min. The buffer was exchanged to PBS, and ⁶⁴Cu-antibody was sterilized using a 0.22 m HT Tuffryn Meanbrane string filter (PALL) in about 167 MBq (about 4.5 mCi) ⁶⁴Cu-antibody with ˜95% average decay-corrected radiochemical yield (RCY). iTLC demonstrated >99% radiochemical purity of ⁶⁴Cu-antibody.

### Example 5—Imaging Experiments

The working examples of this disclosure provide on-demand immolating linker for in vivo radionuclide imaging and therapy. Recent years have seen an increase in the use of antibodies and other biologicals for in vivo use. A key defining factor of these molecules is their long circulation times. While this is desirable in antagonists (blocking a biological function), it is less desirable in radionuclide-based applications as long circulation times add i) unnecessary radiation and ii) decrease the SNR. For example, it is estimated that a single 130 MBq Cu-64 dose of trastuzumab constitutes a significant amount of the annually allowed radiation dose thus limiting the number of scans per year. Given the need for multiple other imaging tests per patient, it is thus desirable to reduce unnecessary radiation. Using the approach described here, the radionuclide-chelate can be cleaved from the antibody and excreted within a short time frame once imaging is complete. The method thus represents a new approach to dose reduction in nuclear imaging and complements alternative approaches such as pre-targeting (e.g., as discussed herein).

Steady development of click reactions over the last 20 years has led to an expanding gamut of applications, including a range of radiolabeling techniques, where the tools have become a method of choice since their first description. The present examples show an application of click tools not to deliver labels but rather to remove them via bioorthogonal bond cleavage. Whereas past investigations of bioorthogonal ligation reactions have indicated a need for rate constants>50,000 M⁻¹s⁻¹ for efficient performance in vivo, here, unexpectedly efficient scission was observed in spite of significantly slower reaction kinetics, on the order of 100-400 M⁻¹s⁻¹ for the Tz scissors click reaction with C₂TCO. The strategy chosen allowed efficient, irreversible cleavage, while using readily achievable concentrations of reagents. The SENIT reagents thus achieved an advantageous combination of high intrinsic biochemical stability (C₂TCO-DOTA, >97% intact at 48 hrs) and facile, high-yielding reactivity in tetrazine-triggered cleavage. The Tz-C₂TCO cleavage was >95% complete in minutes. The conjugates of the present working examples are useful for imaging and for theranostic use to improve signal-to-noise ratios, reduce unnecessary radiation, and allow multiplexed nuclear imaging.

### Other Embodiments

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

What is claimed is:

1. A compound of Formula (I): 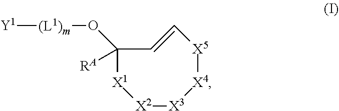
or a pharmaceutically acceptable salt thereof, wherein:
X¹ is selected from O and CR^{A}R¹;
X² is selected from O and CR^{A}R²;
X³ is selected from O and CR^{A}R³;
X⁴ is selected from O and CR^{A}R⁴;
X⁵ is selected from O and CR^{A}R⁵;
each R^{A} is independently selected from H, OH, C₁₋₃ alkyl, and C₁₋₃ haloalkyl;
R¹, R², R³, R⁴, and R⁵ are each independently selected from H, halo, C₁₋₆ alkyl, C₁₋₆ haloalkyl, C₁₋₃ alkoxy, C₁₋₃ haloalkoxy, and a moiety of formula (L²)ₙ-(Y²)ₒ—(Y³)ₚ;
or R¹ and R², together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(═O)OH, C(═O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L²)ₙ-(Y²)ₒ—(Y³)ₚ;
or R² and R³, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(═O)OH, C(═O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L²)ₙ-(Y²)ₒ—(Y³)ₚ;
or R³ and R⁴, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(═O)OH, C(═O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L²)ₙ-(Y²)ₒ—(Y³)ₚ;
or R⁴ and R⁵, together with the carbon atoms to which they are attached, form C₆₋₁₀ aryl, C₃₋₁₀ cycloalkyl, 5-14 membered heteroaryl, or 4-10 membered heterocycloalkyl ring, each of which is optionally substituted with 1 or 2 substituents independently selected from OH, NH₂, C(═O)OH, C(═O)C₁₋₃ alkoxy, C₁₋₃ alkyl, C₁₋₃ haloalkyl, C₁₋₃ alkoxy, C₁₋₃ haloalkoxy, HO—C₁₋₃ alkylene, NH₂—C₁₋₃ alkylene, and (L²)ₙ-(Y²)ₒ—(Y³)ₚ;
each L¹ is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(═O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
each L² is independently selected from N(R^{N}), O, C(═O), S, S(═O), S(═O)₂, C₁₋₆ alkylene, C₃₋₇ cycloalkylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, an amino acid, a self-immolative group, and a moiety formed by a click reaction, each of which is optionally substituted with 1 or 2 substituents independently selected from OH, NH₂, C(═O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
each x is independently an integer from 1 to 2,000;
each R^{N} is independently selected from H, C₁₋₃ alkyl, and C₁₋₃ haloalkyl;
Y¹ is selected from NH₂, OH, C(═O)OH, a protected amino group, a protected hydroxyl group, a protected carboxyl group, a reactive chemical group (e.g., a chemical group reactive with an affinity ligand), and an affinity ligand;
m is an integer from 1 to 20;
n is an integer from 1 to 20;
o is 0, 1, 2, or 3;
p is 0, 1, 2, or 3;
Y² is a chelating moiety; and
Y³ is a radiotracer,
provided that at least one of o and p is other than 0.

2. The compound of claim 1, wherein comprising any one of the following moieties: 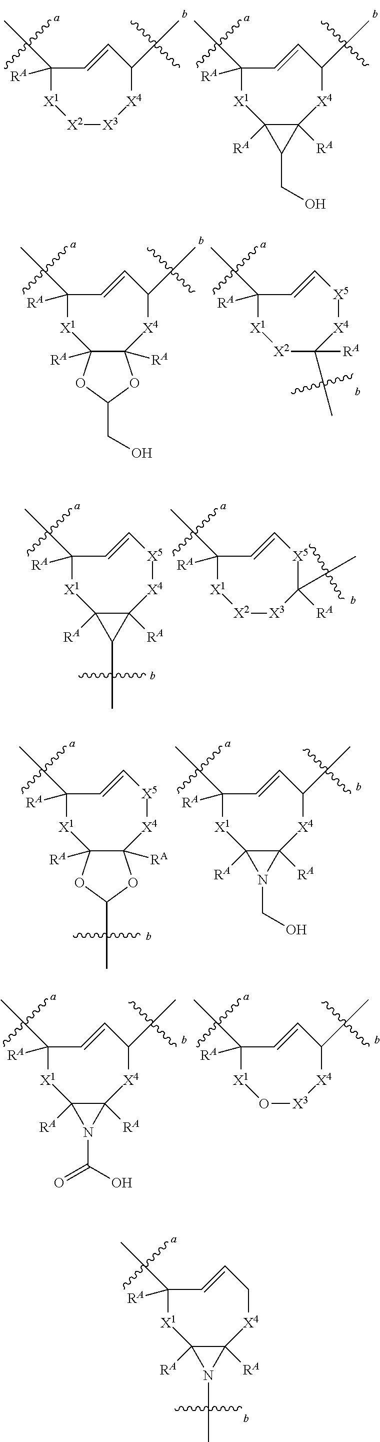
wherein a indicates a point of attachment to a moiety of formula O-(L¹)ₙ-Y¹, and b indicates a point of attachment to a moiety of formula (L²)ₙ-(Y²)ₒ—(Y³)ₚ.

3. The compound of claim 1, wherein the compound of Formula (I) is selected from any one of the following formulae: 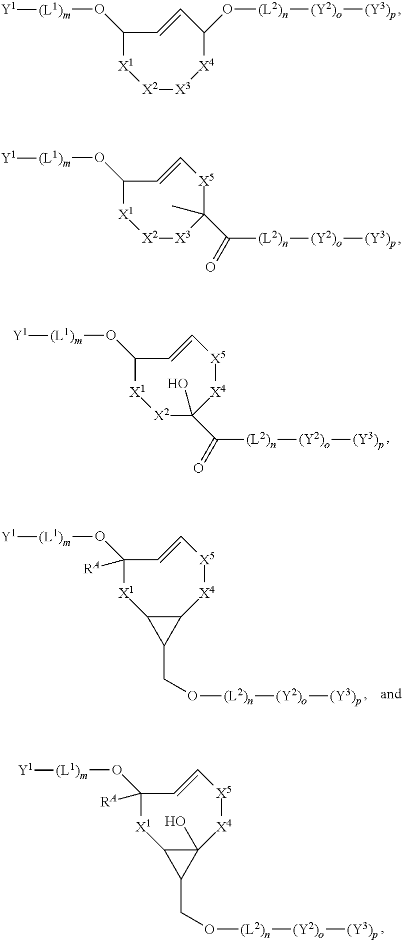
or a pharmaceutically acceptable salt thereof.

4. The compound of claim 1, wherein the compound of Formula (I) has formula: 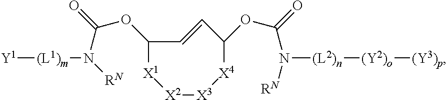
or a pharmaceutically acceptable salt thereof.

5. The compound of claim 1, wherein the compound of Formula (I) is selected from any one of the following formulae: 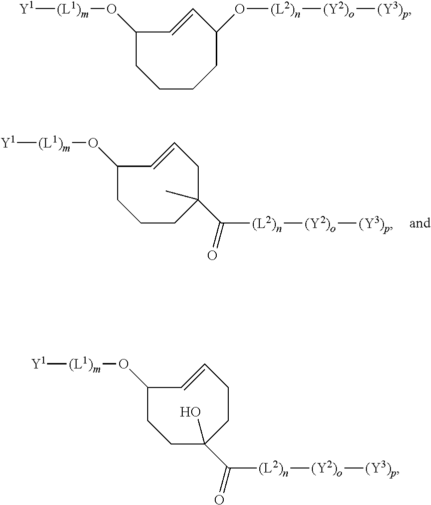
or a pharmaceutically acceptable salt thereof.

6. The compound of claim 1, wherein
o is 1;
p is 1;
the chelating moiety is selected from the group consisting of 1,4,7-triazacyclononanetriacetic acid (NOTA), 1,4,7,10-tetraazacyclododecane-1,4,7,10-tetraacetic acid (DOTA), 1,4,7-triazacyclononane-1-glutaric acid-4,7-diacetic acid (NODAGA), ethylene diamine tetra-acetic acid (EDTA), diethylene triaminepentaacetic acid (DTPA), cyclohexyl-1,2-diaminetetraacetic acid (CDTA), ethyleneglycol-O,O′-bis(2-aminoethyl)-N,N,N′,N′-tetraacetic acid (EGTA), N,N-bis(hydroxybenzyl)-ethylenediamine-N,N′-diacetic acid (HBED), triethylene tetramine hexaacetic acid (TTHA), hydroxyethyidiamine triacetic acid (HEDTA), and 1,4,8,11-tetraazacyclotetradecane-N,N′,N″,N′″-tetraacetic acid (TETA), 1,4,7,10-tetraaza-1,4,7,10-tetra-(2-carbamoyl methyl)-cyclododecane (TCMC), and desferrioxamine B (DFO); and
the radiotracer is selected from ³H, ¹¹C, ¹⁴C, ¹⁸F, ³²P, ³⁵S, ³⁶Cl, ⁴⁴Sc, ⁴⁷Sc, ⁵¹Cr, ⁵²Fe, ^{52g}Mn, ⁵⁷Co, ⁵⁸Co, ⁵⁹Fe, ⁶⁴Cu, ⁶⁷Cu, ⁶⁷Ga, ⁶⁸Ga, ⁷⁵Se, ⁷⁶Br, ⁷⁷Br, ⁸²Rb, ⁸⁶Y, ⁸⁹Zr, ⁹⁰Y, ^{99m}Tc, ¹¹¹In, ^{114m}In, ¹²³I, ¹²⁴I, ¹²⁵I, ¹³¹I, ¹³³Xe, ¹⁵²Eu, ¹⁵³Sm, ¹⁶⁶Ho, ¹⁷⁷Lu, ¹⁸⁶Re, ¹⁸⁸Re, ²⁰¹Tl, ²⁰³Pb, ²¹⁰At, ²¹¹At, ²¹²Bi, ²¹²Pb, ²¹²Bi, ²¹³Bi, ²²³Ra, ²²⁵Ac, and ²²⁷Th.

7. Thy compound of claim 1, wherein Y¹ is the affinity ligand is selected from an antibody, an antibody fragment, a fusion antibody, a chimeric antibody, a nanobody, an engineered antibody, a nanoparticle, an aptamers, an oligo, a peptide, a protein, or a small-molecule.

8. The compound of claim 1, wherein
o is 1 and p is 0; and
Y² has any one of the following formulae: 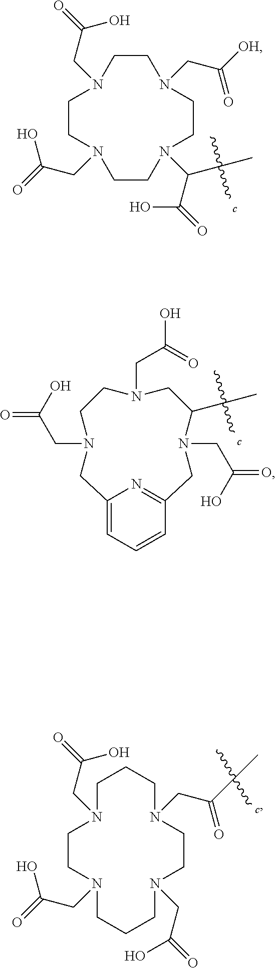 [SMILES: *C(C(=O)O)N1CCN(CC(=O)O)CCN(CC(=O)O)CCN(CC(=O)O)CC1 ; *C1CN(CC(=O)O)CCN(CC(=O)O)Cc2cccc(n2)CN1CC(=O)O ; *C(=O)CN1CCCN(CC(=O)O)CCN(CC(=O)O)CCCN(CC(=O)O)CC1] 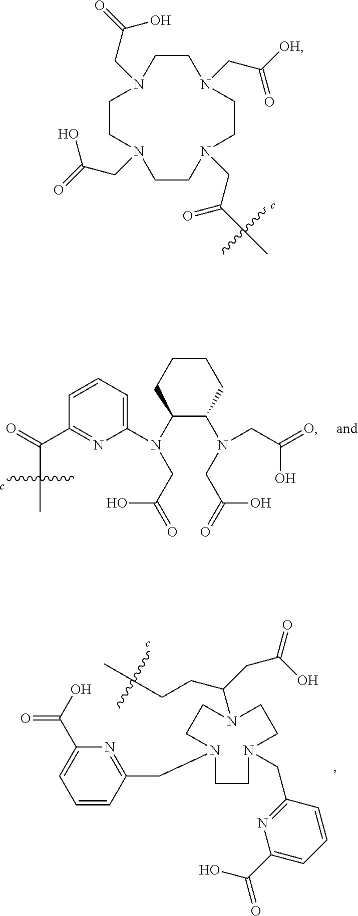 [SMILES: *C(=O)CN1CCN(CC(=O)O)CCN(CC(=O)O)CCN(CC(=O)O)CC1 ; *C(=O)c1cccc(N(CC(=O)O)[C@H]2CCCC[C@@H]2N(CC(=O)O)CC(=O)O)n1 ; *CCC(CC(=O)O)N1CCN(Cc2cccc(C(=O)O)n2)CCN(Cc2cccc(C(=O)O)n2)CC1]
wherein c indicates a point of attachment to L².

9. The compound of claim 1, wherein the compound of Formula (I) has formula: 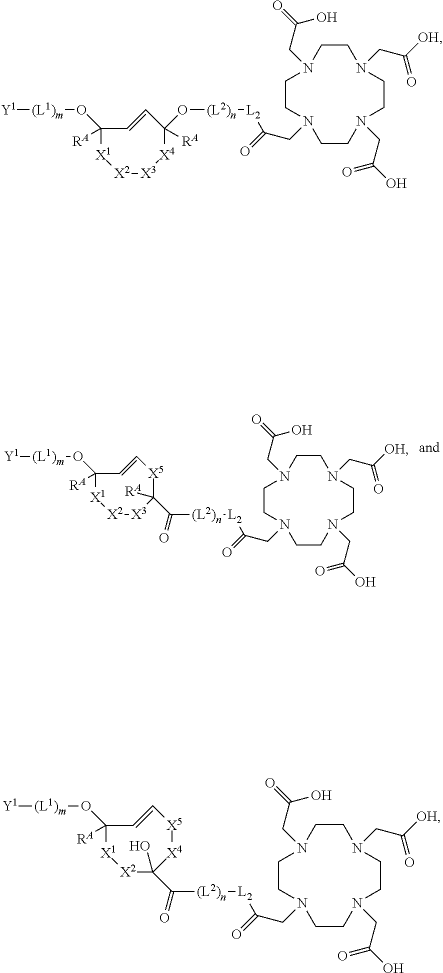
or a pharmaceutically acceptable salt thereof.

10. The compound of claim 1, wherein
o is 1 and p is 1, and
the moiety Y²-Y³ has any one of the following formulae: 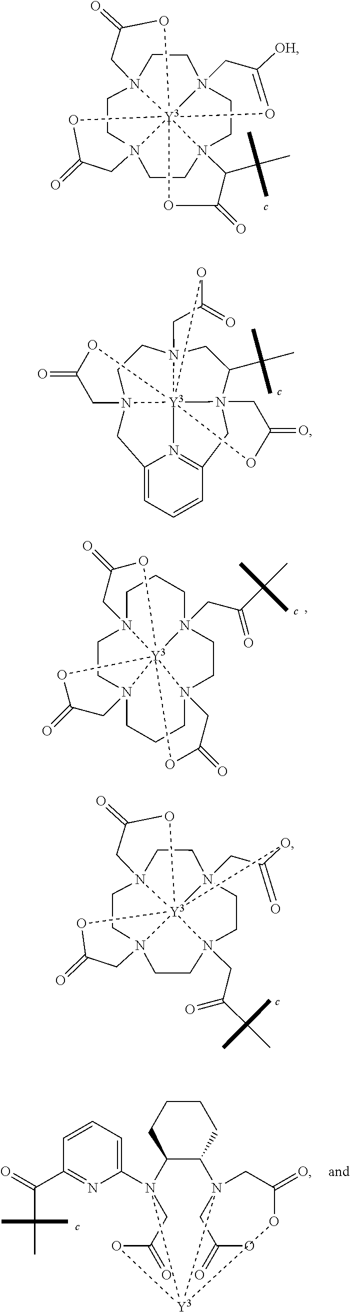 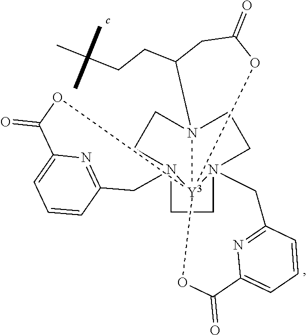
wherein c indicates a point of attachment to L².

11. The compound of claim 1, wherein the compound of Formula (I) has any one of the following formulae: 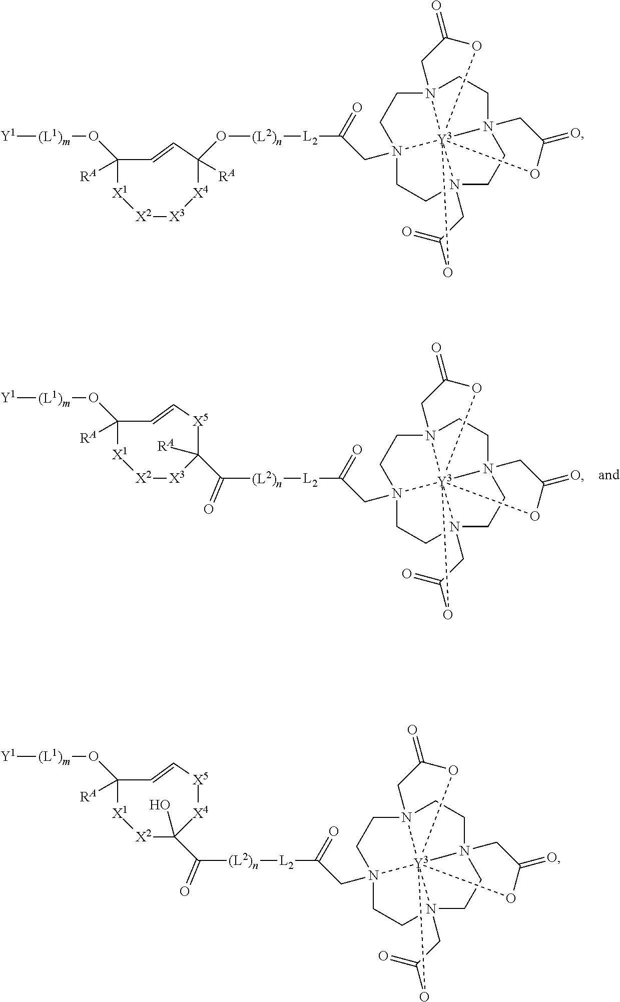
or a pharmaceutically acceptable salt thereof.

12. The compound of claim 1, selected from any one of the following compounds: 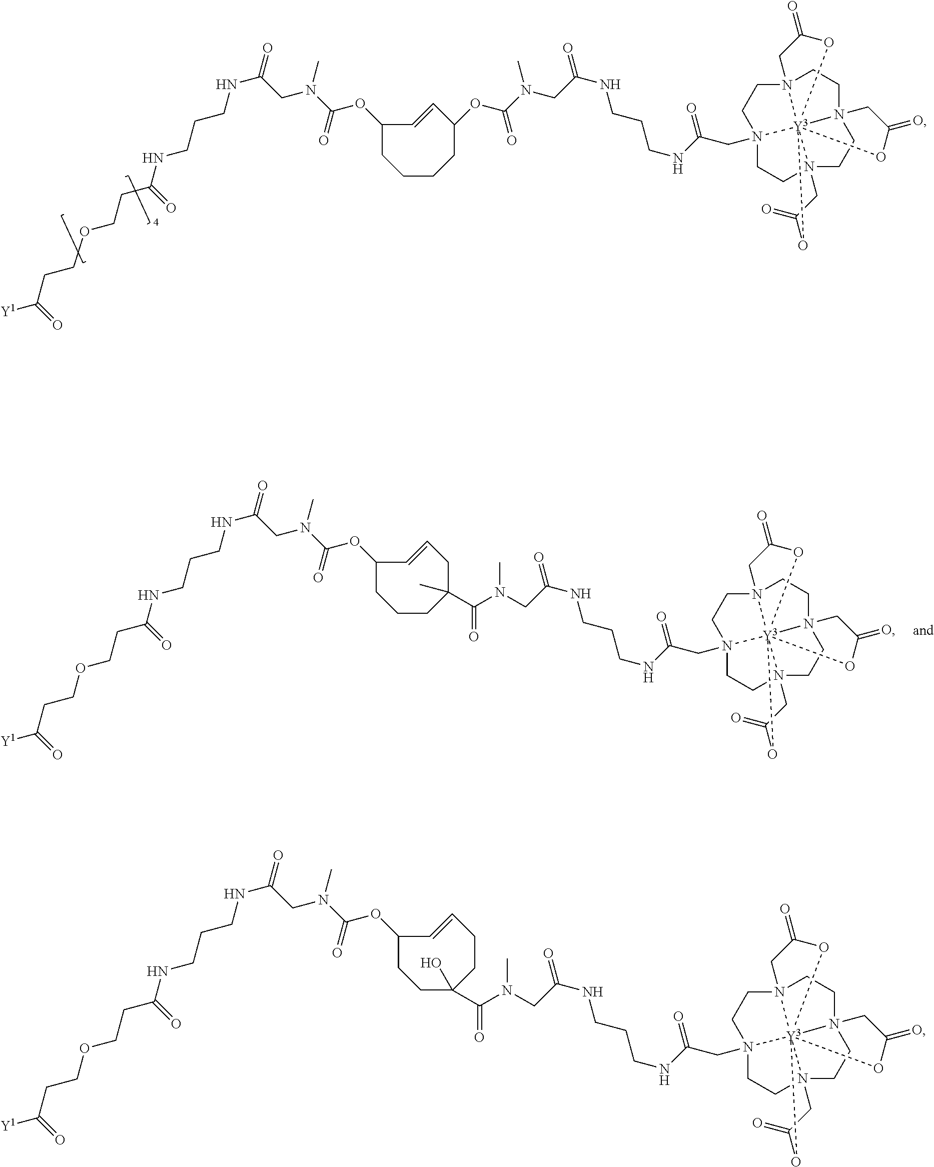
or a pharmaceutically acceptable salt thereof, wherein Y¹ is an affinity ligand.

13. The compound of claim 1, selected from any one of the following compounds: 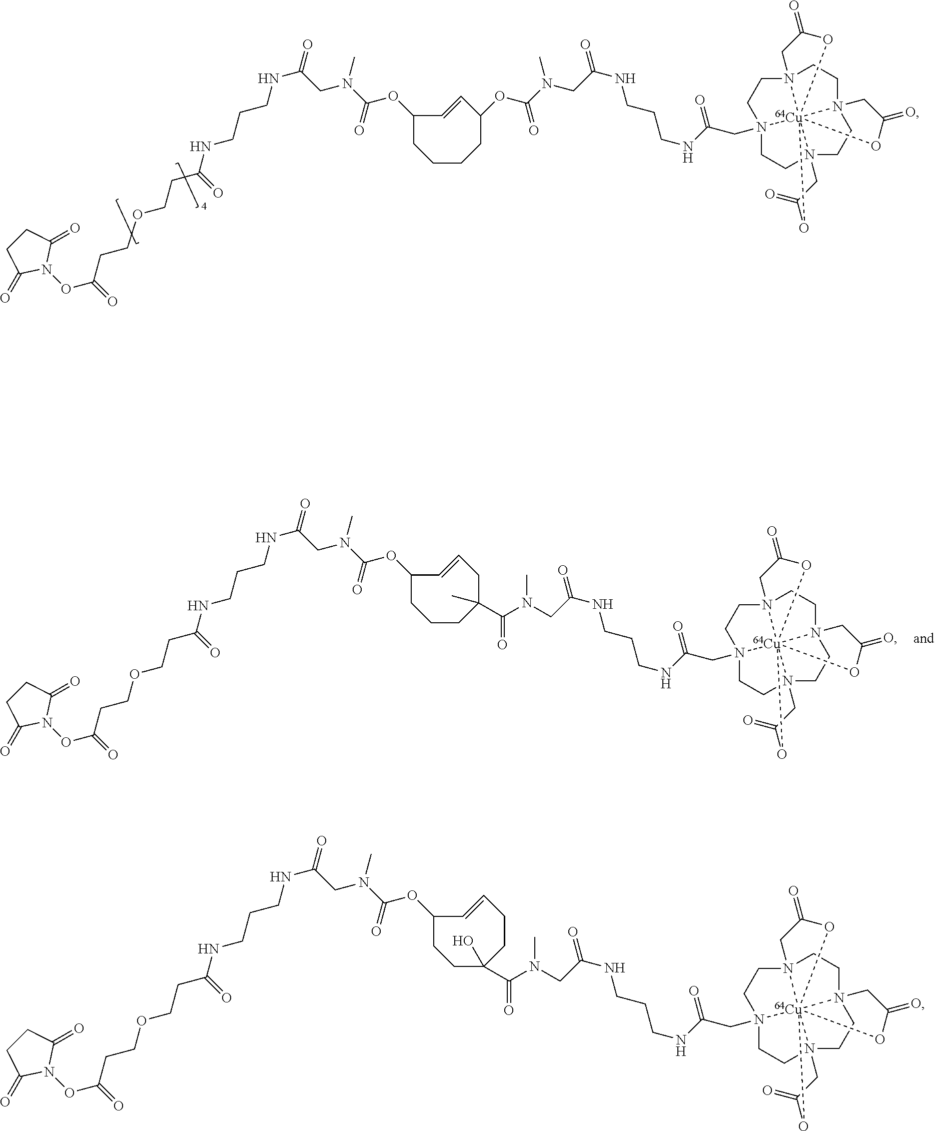
or a pharmaceutically acceptable salt thereof.

14. The compound of claim 1, selected from any one of the following compounds: 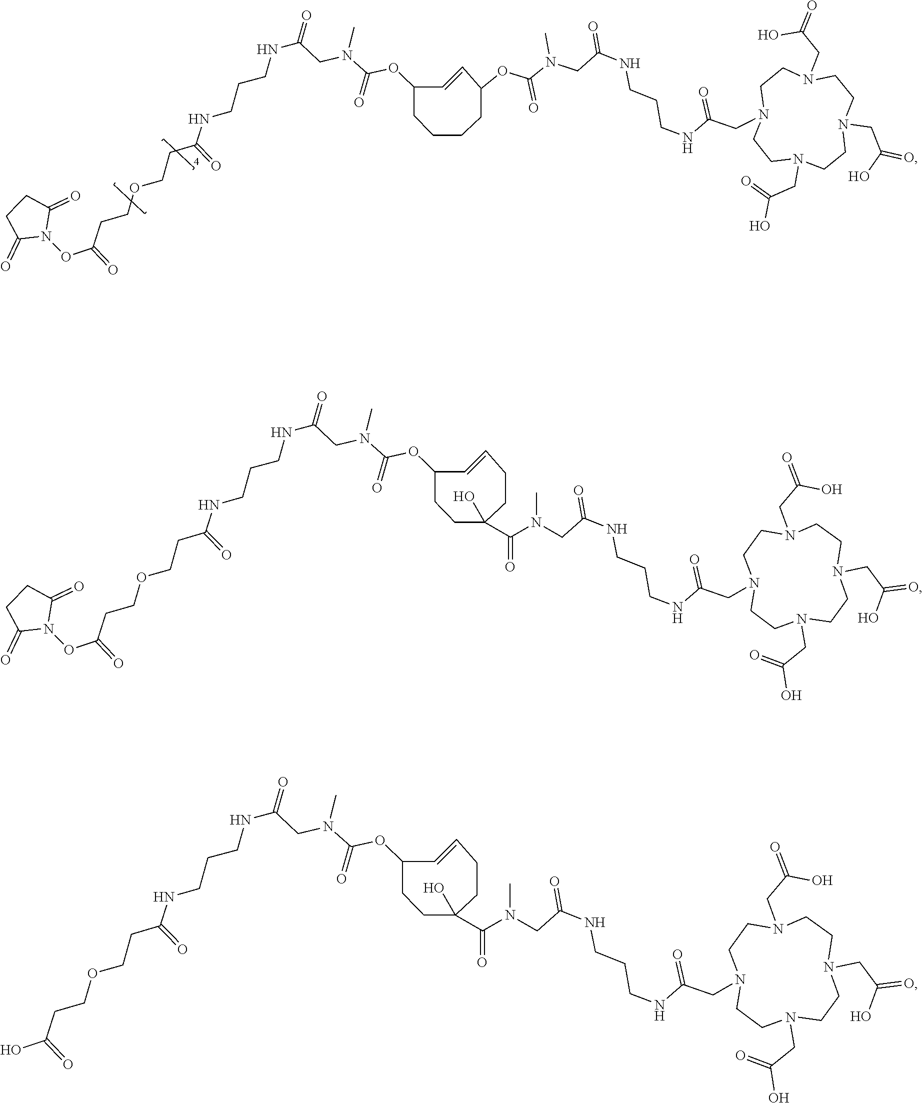 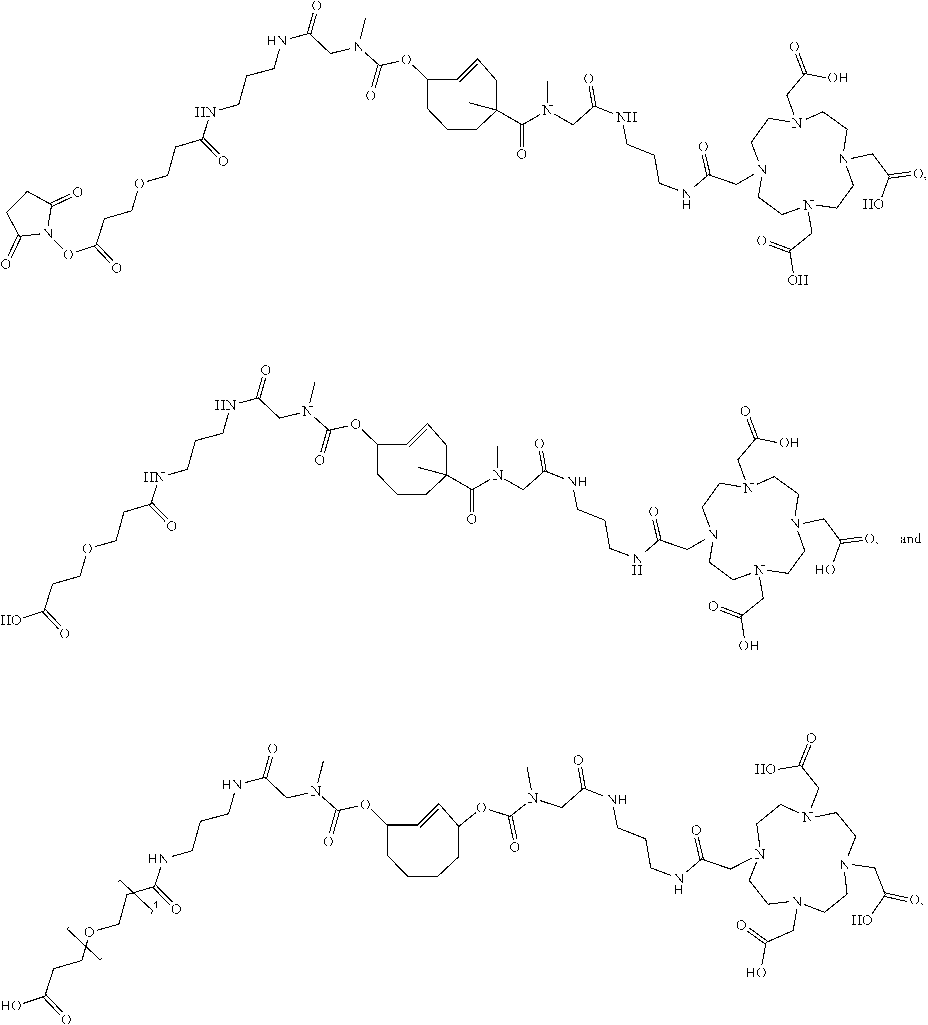
or a pharmaceutically acceptable salt thereof.

15. A pharmaceutical composition comprising a compound of claim 1, or a pharmaceutically acceptable salt thereof, and a pharmaceutically acceptable carrier.

16. A method of imaging a cell or tissue of a subject, the method comprising:
(i) administering to the subject in need thereof a compound of claim 1, or a pharmaceutically acceptable salt thereof, wherein
Y¹ is an affinity ligand; and
Y³ is a PET and/or imagable SPECT imagable radiotracer; and
(ii) waiting a sufficient amount of time to allow the affinity ligand Y¹ within the compound of Formula (I) to bind to its biological target within the cell or tissue of the subject to be imaged.

17. The method of claim 16, wherein the PET imagable radioisotope is selected from ¹¹C, ¹⁸F, ⁴⁴Sc, and, ⁶⁴Cu, ⁶⁸Ga, ⁸²Rb, ⁸⁶Y, ⁸⁹Zr, ¹²³I, ¹²⁴I, ¹²⁵I and ¹³¹I.

18. The method of claim 16, wherein the SPECT imagable radioisotope is selected from ⁶⁷Ga, ^{99m}Tc, ¹¹¹In, ^{114m}In, ¹²³I, ¹³³Xe, ¹⁷⁷Lu, and ²⁰¹Tl.

19. The method of claim 16, comprising a step of:
(iii), after (ii), imaging the subject with an imaging technique selected from positron emission tomography (PET) imaging, positron emission tomography with computer tomography (PET/CT) imaging, positron emission tomography with magnetic resonance (PET/MRI) imaging, and single-photon emission computerized tomography (SPECT) imaging.

20. The method of claim 19, comprising a step of:
(iv), after (iii), administering to the subject a compound of Formula (II): 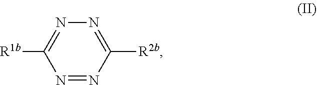
or a pharmaceutically acceptable salt thereof, wherein
R^{1b} and R^{2b} are each independently selected from H, C₁₋₆ alkyl, C₆₋₁₀ aryl, 5-6-membered heteroaryl, C₃₋₁₀ cycloalkyl, 4-7-membered heterocycloalkyl, wherein said C₁₋₆ alkyl, C₆₋₁₀ aryl, 5-6-membered heteroaryl, C₃₋₁₀ cycloalkyl, and 4-7-membered heterocycloalkyl are each optionally substituted with 1, 2, 3, or 4 substituents independently selected from R^{2c};
each R^{2c} is independently selected from halo, CN, NO₂, C₁₋₆ alkyl, C₁₋₆ haloalkyl, C₂₋₆ alkenyl, C₂₋₆ alkynyl, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}; wherein said C₁₋₆ alkyl, C₂₋₆ alkenyl, and C₂₋₆ alkynyl are each optionally substituted with 1, 2 or 3 substituents independently selected from CN, NO₂, C₁₋₆ alkyl, C₁₋₆ haloalkyl, C₂₋₆ alkenyl, C₂₋₆ alkynyl, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1};
each R^{a1}, R^{b1}, R^{c1}, and R^{d1} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆-10 aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene are optionally substituted with 1, 2, 3, 4, or 5 substituents independently selected from R^{g}; and
each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

21. A method of treating a disease or condition in a subject, the method comprising:
(i) administering to the subject in need thereof a therapeutically effective amount of a compound of claim 1, or a pharmaceutically acceptable salt thereof, wherein:
Y¹ is an affinity ligand; and
Y³ is a toxic radiotracer.

22. The method of claim 21, wherein the toxic radiotracer is selected from ⁴⁷Sc, ⁹⁰Y, ^{114m}In, ¹³¹I, ¹⁷⁷Lu, ¹⁸⁶Re, ¹⁸⁸Re, ²¹¹At, ²¹²Bi, ²¹²Pb, ²¹²Bi, ²¹³Bi, ²²³Ra, ²²⁵Ac, and ^{227Th}.

23. The method of claim 21, comprising a step of:
(ii), after (i), waiting a sufficient amount of time to allow the affinity ligand Y¹ within the compound of Formula (I) to bind to its biological target within the cell or tissue affected by the disease within the subject.

24. The method of claim 23, comprising a step of:
(iii) imaging the subject with an imaging technique selected from positron emission tomography (PET) imaging, positron emission tomography with computer tomography (PET/CT) imaging, positron emission tomography with magnetic resonance (PET/MRI) imaging, and single-photon emission computerized tomography (SPECT) imaging.

25. The method of claim 24, comprising a step of:
(iv) after (iii), administering to the subject a compound of Formula (II): 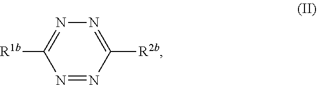
or a pharmaceutically acceptable salt thereof, wherein
R^{1b} and R^{2b} are each independently selected from H, C₁₋₆ alkyl, C₆₋₁₀ aryl, 5-6-membered heteroaryl, C₃₋₁₀ cycloalkyl, 4-7-membered heterocycloalkyl, wherein said C₁₋₆ alkyl, C₆₋₁₀ aryl, 5-6-membered heteroaryl, C₃₋₁₀ cycloalkyl, and 4-7-membered heterocycloalkyl are each optionally substituted with 1, 2, 3, or 4 substituents independently selected from R^{2c};
each R^{2c} is independently selected from halo, CN, NO₂, C₁₋₆ alkyl, C₁₋₆ haloalkyl, C₂₋₆ alkenyl, C₂₋₆ alkynyl, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1} NR^{c1}C(O)OR^{a1}; wherein said C₁₋₆ alkyl, C₂₋₆ alkenyl, and C₂₋₆ alkynyl are each optionally substituted with 1, 2 or 3 substituents independently selected from CN, NO₂, C₁₋₆ alkyl, C₁₋₆ haloalkyl, C₂₋₆ alkenyl, C₂₋₆ alkynyl, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1};
each R^{a1}, R^{b1}, R^{c1}, and R^{d1} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆-10 aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene are optionally substituted with 1, 2, 3, 4, or 5 substituents independently selected from R^{g}; and
each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.
